SEQUENCE LISTING (120 sequences)

SEQ ID NO: 1 - DNA, 30 bp, Artificial
  Synthetic
aagcttggatccagatcttctagagaattc

SEQ ID NO: 2 - DNA, 933 bp, Artificial
  Synthetic
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaaccttttttttatcatcattattagcttactttcataattgcgactggttccaattgacaagcttttgattttaacgacttttaacgacaacttgagaagatcaaaaaacaactaattattcgaaacg

SEQ ID NO: 3 - DNA, 31 bp, Artificial
  Primer
ttaggatccaacatccaaagacgaaaggttg

SEQ ID NO: 4 - DNA, 38 bp, Artificial
  Primer
ttaggatcccgtttcgaataattagttgttttttgatc

SEQ ID NO: 5 - DNA, 500 bp, Artificial
  Synthetic
tcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgcaagcttcaacgatgccaaaagggtataataagcgtcatttgcagcattgtgaagaaaactatgtggcaagccaagcctgcgaagaatgtattttaagtttgactttgatgtattcacttgattaagccataattctcgagtatctatgattggaagtatgggaatggtgatacccgcattcttcagtgtcttgaggtctcctatcagattatgcccaactaaagcaaccggaggaggaga

SEQ ID NO: 6 - DNA, 39 bp, Artificial
  Primer
ttatctagatcaagaggatgtcagaatgccatttgcctg

SEQ ID NO: 7 - DNA, 31 bp, Artificial
  Primer
ttatctagatctcctcctccggttgctttag

SEQ ID NO: 8 - DNA, 2656 bp, Artificial
  Synthetic
agatctcctgatgactgactcactgataataaaaatacggcttcagaatttctcaagactacactcactgtccgacttcaagtatgacatttcccttgctacctgcatacgcaagtgttgcagagtttgataattccttgagtttggtaggaaaagccgtgtttccctatgctgctgaccagctgcacaacctgatcaagttcactcaatcgactgagcttcaagttaatgtgcaagttgagtcatccgttacagaggaccaatttgaggagctgatcgacaacttgctcaagttgtacaataatggtatcaatgaagtgattttggacctagatttggcagaaagagttgtccaaaggatcccaggcgctagggttatctataggaccctggttgataaagttgcatccttgcccgctaatgctagtatcgctgtgcctttttcttctccactgggcgatttgaaaagtttcactaatggcggtagtagaactgtttatgctttttctgagaccgcaaagttggtagatgtgacttccactgttgcttctggtataatccccattattgatgctcggcaattgactactgaatacgaactttctgaagatgtcaaaaagttccctgtcagtgaaattttgttggcgtctttgactactgaccgccccgatggtctattcactactttggtggctgactcttctaattactcgttgggcctggtgtactcgtccaaaaagtctattccggaggctataaggacacaaactggagtctaccaatctcgtcgtcacggtttgtggtataaaggtgctacatctggagcaactcaaaagttgctgggtatcgaattggattgtgatggagactgcttgaaatttgtggttgaacaaacaggtgttggtttctgtcacttggaacgcacttcctgttttggccaatcaaagggtcttagagccatggaagccaccttgtgggatcgtaagagcaatgctccagaaggttcttataccaaacggttatttgacgacgaagttttgttgaacgctaaaattagggaggaagctgatgaacttgcagaagctaaatccaaggaagatatagcctgggaatgtgctgacttattttattttgcattagttagatgtgccaagtacggtgtgacgttggacgaggtggagagaaacctggatatgaagtccctaaaggtcactagaaggaaaggagatgccaagccaggatacaccaaggaacaacctaaagaagaatccaaacctaaagaagtcccttctgaaggtcgtattgaattgtgcaaaattgacgtttctaaggcctcctcacaagaaattgaagatgcccttcgtcgtcctatccagaaaacggaacagattatggaattagtcaaaccaattgtcgacaatgttcgtcaaaatggtgacaaagcccttttagaactaactgccaagtttgatggagtcgctttgaagacacctgtgttagaagctcctttcccagaggaacttatgcaattgccagataacgttaagagagccattgatctctctatagataacgtcaggaaattccatgaagctcaactaacggagacgttgcaagttgagacttgccctggtgtagtctgctctcgttttgcaagacctattgagaaagttggcctctatattcctggtggaaccgcaattctgccttccacttccctgatgctgggtgttcctgccaaagttgctggttgcaaagaaattgtttttgcatctccacctaagaaggatggtacccttaccccagaagtcatctacgttgcccacaaggttggtgctaagtgtatcgtgctagcaggaggcgcccaggcagtagctgctatggcttacggaacagaaactgttcctaagtgtgacaaaatatttggtccaggaaaccagttcgttactgctgccaagatgatggttcaaaatgacacatcagccctgtgtagtattgacatgcctgctgggccttctgaagttctagttattgctgataaatacgctgatccagatttcgttgcctcagaccttctgtctcaagctgaacatggtattgattcccaggtgattctgttggctgtcgatatgacagacaaggagcttgccagaattgaagatgctgttcacaaccaagctgtgcagttgccaagggttgaaattgtacgcaagtgtattgcacactctacaaccctatcggttgcaacctacgagcaggctttggaaatgtccaatcagtacgctcctgaacacttgatcctgcaaatcgagaatgcttcttcttatgttgatcaagtacaacacgctggatctgtgtttgttggtgcctactctccagagagttgtggagattactcctccggtaccaaccacactttgccaacgtacggatatgcccgtcaatacagcggagttaacactgcaaccttccagaagttcatcacttcacaagacgtaactcctgagggactgaaacatattggccaagcagtgatggatctggctgctgttgaaggtctagatgctcaccgcaatgctgttaaggttcgtatggagaaactgggacttatttaattatttagagattttaacttacatttagattcgatagatct

SEQ ID NO: 9 - DNA, 32 bp, Artificial
  Primer
ttagaattcggatctcctgatgactgactcac

SEQ ID NO: 10 - DNA, 41 bp, Artificial
  Primer
ttagaattcggatctatcgaatctaaatgtaagttaaaatc

SEQ ID NO: 11 - DNA, 1056 bp, Artificial
  Synthetic
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcttacgtagaattcgctcaggttcaattgcaagaatccggtccaggtttggttaagccatctgagactttgtccttgacttgtactgtttccggtggttccatttcctcttaccactggtcctggattagacagccaccaggtaaaggtttggagtggatcggttacatctactactccggttccactaactacaacccatccttgaagaacagagttactatctccgttgacacttccaagaaccagttctccttgaacttgagatccgttactgctgctgacactgctgtttactactgtgctagaggtacttacggtccagctggtgatgctttcgatatttggggtcagggtactactgttacagtttcctctggtggtggtggatcaggtggtggtggttctggtggtggtggatctgatattcaaatgactcagtccccatccactttgtccgcttctattggtgatagagttacaattacttgtagagcttccgagggtatctaccattggttggcttggtatcaacagaagccaggtaaggctccaaagttgttgatctacaaggcttcctctttggcttctggtgctccatctagattttctggttccggttctggtactgactttactttgactatctcctccttgcaacctgacgacttcgctacttactattgtcagcagtactccaactacccattgactttcggtggtggtactaagttggagatcaagagagctgctgctggtggtggtggttcacatcatcatcatcatcactag

SEQ ID NO: 12 - DNA, 35 bp, Artificial
  Primer
ttaagatctatgagatttccttcaatttttactgc

SEQ ID NO: 13 - DNA, 36 bp, Artificial
  Primer
ttaagatctctagtgatgatgatgatgatgtgaacc

SEQ ID NO: 14 - DNA, 30 bp, Artificial
  Synthetic
aagcttggatccactagttctagagaattc

SEQ ID NO: 15 - DNA, 487 bp, Artificial
  Synthetic
ttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 16 - DNA, 30 bp, Artificial
  Primer
ttaggatccttttttgtagaaatgtcttgg

SEQ ID NO: 17 - DNA, 37 bp, Artificial
  Primer
ttaggatcctgtgttttgatagttgttcaattgattg

SEQ ID NO: 18 - DNA, 855 bp, Artificial
  Synthetic
cccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 19 - DNA, 33 bp, Artificial
  Primer
ttagaattccccacacaccatagcttcaaaatg

SEQ ID NO: 20 - DNA, 31 bp, Artificial
  Primer
ttagaattctcagtcctgctcctcggccacg

SEQ ID NO: 21 - DNA, 600 bp, Artificial
  Synthetic
gacaataagaagaaaaaaaaagaaaagcggtgggggagggattattaaataaggattatgtaaccccagggtaccgttctatacatatttaaggattatttaggacaatcgatgaaatcggcatcaaactggatgggagtatagtgtccggataatcggataaatcatcttgcgaggagccgcttggttggttggtgagaggagtgaaatatgtgtctcctcacccaagaatcgcgatatcagcaccctgtgggggacactattggcctccctcccaaaccttcgatgtggtagtgctttattatattgattacattgattacatagctaaaccctgcctggttgcaagttgagctccgaattccaatattagtaaaatgcctgcaagataacctcggtatggcgtccgaccccgcttaattattttaactcctttccaacgaggacttcgtaatttttgattagggagttgagaaacggggggtcttgatacctcctcgatttcagatcccaccccctctcagtcccaagtgggacccccctcggccgtgaaatgcgcgcactttagtttttttcgcatgtaaacgccggtgtccgtca

SEQ ID NO: 22 - DNA, 39 bp, Artificial
  Primer
ttatctagagacaataagaagaaaaaaaaagaaaagcgg

SEQ ID NO: 23 - DNA, 29 bp, Artificial
  Primer
ttatctagatgacggacaccggcgtttac

SEQ ID NO: 24 - DNA, 1482 bp, Komagataella pastoris
atgcttgctgagattggggactatttcaggacaaatagtagcaatggattcagttctagctgctataacaaagagaagctttcaggagacctcttggagtccgattttgtgaattcatcattcgatgacacaacactccactctatgctatcttcttcaataaatgctgatccggagcaatcccagaaaccacaacaaagacacaagaatcaatccagttgccatcaggaactcaagattgaaacagaagatcagcaagaaccgtatctattgcctgatatgcttaccaaaaacatattcaactcacagtcttctatgtttgtctactccagcatcccacaaataagctcagtgcaatcgtttatagacacaaacaccttttccgctgttgatggtggtgccgccccggaaggaaattcattgtctgtggactcaactttaggtctatcttctgaccaatcatttgagaagctatactcttacgaccaaaactttgagaaagaaccacaatcgccagcatgtcccttagtaaaatgtgacccagtaagcatcatgaaaacatccacaaactcattggagacagctgtcaacaccaaaagacgaatgtttcagcttagaaaccaaaatgaaattgaaagtctcaatgtttctagtgacatcgttgttgcgacatcaaaatttctcaatcattcatggatcccgctgatccgtggacgtaatttcggaggaagcaactgcaaaccgcccaagaaaccattatttcctggagcgcgctatgttactgcccgtctacgattggaatacagtagctgtaaagatatgtgtctaccggaatggaacgagtatgaactcgaagacagtagaagaatcattcgtattgagagactgttcgactccaatgaaatcgtagcctccttttccattgtcgggtctgctgtagagaacccggaaactaggcctgtattcaatcctaatgtcaaagtgttggaagtttcgtgtctaagatgtcttaccaacaacaacgagtctgacgaggagaactgtgtcaacaaagatttggatggccgtaaccgcgatatggttaaggactcatttggatgcaagtactatatcaccagtgttgaagtcataaagattatagaattactggtcggaagctcttcaatctcagatcctcatcaaatgagaaaagaaagagggcgtgtgagatccaacttggctcagttctggtccaaacgcctagtttcttcgtccaggaaaaccatgaaacaaggtttcttgccaacatgcaacgatgattattttgcagagctggcccatcgtattaacacatacgacgtcagaaaaccacgattatttgacaagtgtataaagattttggaatggtctaaacttaagccagctctgcagcgggctatgcaaagttactacatggttcaactggatgaatctagcaacaaaattgcaacagcaaacaattag

SEQ ID NO: 25 - DNA, 55 bp, Artificial
  Primer
caatcaattgaacaactatcaaaacacaatgcttgctgagattggggactatttc

SEQ ID NO: 26 - DNA, 59 bp, Artificial
  Primer
ccgcttttctttttttttcttcttattgtcctaattgtttgctgttgcaattttgttgc

SEQ ID NO: 27 - DNA, 1557 bp, Komagataella pastoris
atgaattcatcttcaactcgtggcctgcaagctatatcactcacaccaactagagttttattcactttgagcactctgttcgccgatacgtcaaaaatgtctgctgagaatggggactatttcaggacaaacagtagcaatgaattcagcaataactgctttaacaaagaaaagctctcggaggacctcttggaggccagttttgtcgattccccaatcgaagacacaataccccactttatgctatcttcttcaataaatgctgattcggaacagcctcaggaaccacagcatcgacacaagaatcaatccagttgccatcgggaactcaagatcgaaacaaacgatcagcaagaaccgtatctgttgcctgatatgcttaccaaaaacatattcaactcacagtcttctatgtttgtctattccagcatcccacaaacaagctcagtgcaatcgtttatagacacaaacattttttccgctgttgatggtggtgccgccccagaagaaaattcattgtctgtggactcaaccttaggtccatcttctgaccaatcatttgaaaagctatactcttacgaccaaaattttgagaaagaaccacaatcgccagcatgtcccttagtaaaatgtgacctagtaggcatcatgaaagcatccacaaactcattggaggtagctgtcaacaccaaaagacaaatgtttcagcttagacaccaaaatgaaattgagagtctctatgtttctagtgacatcgttattgcgacatcaaattttcttgaccattcatggattccgctgatccgtggacgtaatttcggaggaagcaactgcaaaccgcccaagaaaccattatttcctggagcgcgctatgttactgcccgtctacgattggaatacagtagctgtgaagatatgtgtttaccggaatggaacgagtatgaactcaaagacagtagaagaatcattcgtcttgagaggcggttcgactccaatgaaatcgtggcctccttttccattgtcgggtctgctgtagagaaccctgaaactaggcctgcattcaatcctaacgtcaaagtgttggaagtttcgtgtctaaggtgtcttaccaacaacaacgaatctgacgaggagaactgtgtcaacaaagatttggatggccgtgtccacaatatggttaaagactcaattggatgcaagtactatattaccagtgttgaagtcataaagattatagaattactggtcggaagctcttcaatttcagatccccaacaaatgagaaaagaaagaggccgtgtgagatcaaacttggctcagttctggtccaaacatttagtttcttcgtccaggaaaaccaggaaacaacgtttcttgccaacatgcaacgatgattatttcgcagagctggtccatcgtatcaacacttatgacgtgagaaaacctcgactattcgataagagcgtaaagattctggaatggcctaatcttggaccagctttgcaacgggctatccagagttactacatggttcgactggaagaatctagcaactaa

SEQ ID NO: 28 - DNA, 48 bp, Artificial
  Primer
caatcaattgaacaactatcaaaacacaatgaattcatcttcaactcg

SEQ ID NO: 29 - DNA, 65 bp, Artificial
  Primer
ccgcttttctttttttttcttcttattgtcccatttgctgctctacccctgtcctaaacttattg

SEQ ID NO: 30 - DNA, 1572 bp, Komagataella pastoris
atgcggttgtatttagcaactgtaggaactccattcatcggtgcaccgccagtgattgctagtaatgaagaccatagcactgctcacaataacaatagcaacgactttaattatgaatgctataataaagaaaagctctctggggatctcttggaagctgacctttttgatcctccaattgatgatacaatgcactgctccatgttttcgtcatcaacaactgctgacactgagcagctacaaaaaccaaagcatgaatcgcaacacaagaatcccttcgattgttgtcaggaactcaagcttaaaatggccgatcagcaagagttgcatttgttgccggagaaaattaccaagaatatttttaattcagagtcgcctgtgttcatatgcaccaagattccccaaacaagtccggtccagtcgttcgtagatgttaatgcttctttacttgccactaactccgccctgactacaaattcattctctgtagacacaacagttgatatattgtttgagcaaacagttcaagatccatactcttaccgtttaaactctaaggaaacagcacctccatccacagctagcttctttgggactctcgatctgggacccaccgtggaagcatccacaacgttactggaggcagccgtcaactctaaaagacgaatagttgaacttagacagcaaaacagaatcgaggatctcaatttttcaacatcaatgttccttgaccattcacgcataccgttggtgcgtgggcgtagctttggaggaaatagcagcagacctcccaagacgccattaattcctgggacgcggtacgttactgcccgtttaagactggaaaacagcacaagtgaggatatatgtttgccccattggaacaaagcggaactcaaagactctagaagaataatccgtattgaaagaagataccagtccaatgagattgtagcaaccttttccatagttggatcggccatagagcacccaaagactaagcctgcattcgatcctgacgtcaaggttttggaagtctcgtgtctaaaatgtcccaccaacgaaaatgaacctaacgaggagcgatttgtcagtgaatgtacgtcagatgaagcacgagcacaacatgccttaacagaacccctaatgtcaggcttcaacagagaaccgaatggccgtaaccctaaaacggctgggaattcaattggatgcaggtactatattaccagtgtcgaagtcatcaagataatagaactactgattggaagctattcaacctcagaccccgtgcaaaggagaaaagagagaggccgtatacgatctaacttggtgcagttctggtccaaacacctagtcttttcctcgaagaactctatgaagcgccgttccgtgccaacaggcaacgatgcttatcttacggagctggagcatcgtattaacacttatgatgtgagaaaacctcgactatttgataagagtgtcaagattctggaatggtctaaacttggacctgctttgcgtcgggctatccaaagctattacatggtacgactagacgaatccagcaactaa

SEQ ID NO: 31 - DNA, 54 bp, Artificial
  Primer
caatcaattgaacaactatcaaaacacaatgcggttgtatttagcaactgtagg

SEQ ID NO: 32 - DNA, 54 bp, Artificial
  Primer
ccgcttttctttttttttcttcttattgtcttagttgctggattcgtctagtcg

SEQ ID NO: 33 - DNA, 1599 bp, Komagataella pastoris
atgcggttgtctttagcaacacgagaaacaccgtctaccggtacatcgccggtgtttgctggtgacgaagatcatagcagtgccaacaatagcagtagcaacactttcaactatgactactatataaaagaagaactctctggaaatctcctggaaacagccttttttgaatctccgattaatgatacaatgctctgctccatgttatcgtatccaaaaattgctggtactgagaaaatacaagaaccagaccatgaactgcagcaacacaggaatcccttcggttgctgccaggaactcaaagctgaattggccgaacagcaagagctgtatctgctgccaaataaacttaccaagaatatttttaattcagggtctcctgtgttcatatacaccaagatcccacaaacaagctcggttcagtcatttgtggatgttagcaattctttatttgccactgactccgccttgcctacaagttcatttcgtgtagactcaacagtcgacatatcgtttgagcactcaattcaagattcatactgttaccgttcaaactctgcagaaatagcacctccatccacagcactctcttcagggactcacgacctggtatccaccgtggaagcatccacgacgttactggaggcagccgtcaactctaaaagacgaatgcttgaacttagacagcaaaacagaatcgaggatctcagtttttcaacatcaatattccttgaccattcacgcctaccgttggttcgtgggcgtagctttggaggaaatagcagtagacctcccaagacgccattgattcctgggacgcggtacgttactgcccgtttaagactggaaaacagcacaagtgaggatatgtgtttgccccagtggaacaaggcggaactcaaagactctagaagaataatccgtattgaaagaagataccagtctgatgaaattgtcgcttccttttccatagttggatcggccatagagtacccaaagactaagcctgcattcgatcctgacgtcaaggttttggaagtctcgtgtctaaaatgtcccaccaacgaaaatgaacctaacgaggagcgatttgtcagtgaatgtacgtcagatgaagcacgaacacaacatgccttaacagaacccctaacgtcaggcttcaacagagaaccgaatggccataaccctaaaacggctgggaattcaattggatgcaggtactatattaccagtgtcgaagtcatcaagataatagaactactgattggaagctattcaacctcagaccccgtgcaaaggagaaaagagagaggccgtatacgatctaacttggtgcagttctggtccaaacacctagtcttttcctcgaagaactctatgaagcgccgttccgtgccaacaggcaacgatgcttatcttacggagctggagcatcgtattaacacttatgacgtgagaaaacctcgactatttgacaagagtgtcaagattctggaatggtccaaacttgggccagctttgcagcgggctatccaaagctattacatggttcggctagataattccagcgacataactgcaacaacaaatagcagctaa

SEQ ID NO: 34 - DNA, 51 bp, Artificial
  Primer
caatcaattgaacaactatcaaaacacaatgcggttgtctttagcaacacg

SEQ ID NO: 35 - DNA, 53 bp, Artificial
  Primer
ccgcttttctttttttttcttcttattgtcttagctgctatttgttgttgcag

SEQ ID NO: 36 - DNA, 1524 bp, Komagataella pastoris
atgcagctactgtcagcaagttcaagcactccattcgtcgctatgtcgacagtaaacgctgggaacgagtgttatcttaggaccaacaatagtaacagcaacggtatcagcgataaccgcttcagaaaaaaaaagctttctgaggatttgttgaaagccgattttgtcgaagacacagccggtaacccaatgatttaccccatggttttatctgctccaacatatataaaaactgaacatctccaagaattggagcataaacagcaaggatcaaatctgttgtctgaaatgtttaccaaaaatattttcaattcacattctgcaatgtttgtttactctgaaatccctcaaataagttcggttcagtcattcattggttctgacacctctgtccttgttgctgatgcctcttcagaaaaaaattcattgtttgtgtactcaaatactggtatatcttttgatccgtcagatgaggatccatgcccttacgaggctaattttgatccaggacaactattctcagcggtatccgcagtagaagacggttctatggcaacattgacagctcccactatgtcattagaaggtgtcgttaattccaaaaaaggaatatgtcaacttagacagcataacagaatcgagggatttgattcttcccatgaattcaaagtcttgggatcaatgttccttaaccattcgtacgtaccgctggttcgtggacgtagtttcggaggaaacaattgtagacctcccaaagagccattaattcctggaacccggtacgttgctgcccatctaagattggaaaacaatacctgcgaggatatgtgtctcccagaatggaacaagcatgagcttgaagatagtagaagaatcattcgtatagagagaagataccagtcgaatgaaattgtggcctctttttctatagttgggtctgctaaagagaatccacaaacaaagccttcaagcgatcctgacgtgaaagttttggaagtctcgtgtctgaaatgtttcatcaacggcaacgaatctgacgagcaatttgtgactgaatgtcagtcagatgaagcgcattatggcttcacagagttaccaaaacaaaactttagcagaaaatcaaaaggctgcagccacaaaatggcaaataacacgattgcatgccaatactatattaccagcgtcgaagtcatcaagattattgaattactggtcggaggctatacagtttctgatcctcggcagaggaggaaagaaagaggccgcgtgagatcaaacttggctcaattctggtccaaacacttagtttcttcttccaagaaaacgatgaagcgccctgcgatgctagcatgcaacgaggattatcttgcagagctggaacagcgtatcaacacctatgacgtgagaaagccgcgactatttgataagagcgttaagattctcgaatggcctaatcttggaccagctttgcagcgggctattcagagctattacatggtgcaactggaagaatccagcaactag

SEQ ID NO: 37 - DNA, 55 bp, Artificial
  Primer
caatcaattgaacaactatcaaaacacaatgcagctactgtcagcaagttcaagc

SEQ ID NO: 38 - DNA, 55 bp, Artificial
  Primer
ccgcttttctttttttttcttcttattgtcctagttgctggattcttccagttgc

SEQ ID NO: 39 - DNA, 1614 bp, Komagataella pastoris
atgcaattgtttttagcaactctaggaattccgttcaccggcacattaccagccattgctggtaacgaggatcatagcagtgccaacaacaacaatggcaacggtttcagttatgactggcatggaaatgatacgctttttgcggaactcttggaaactgacttttttgatcatccgattgatgacgcaatgctctgctccttgttatcccctccaagagctgctgagactgagcaacagcaagatacagaatgggagcaacaaaaacacacaaatctctttggttgctgccagaaacacaaagttgaaatggccaaacagcaagaactgtatttgctgccagataagcttaccaagaatattttcaattcagagtcgcctgtgttcatatgcaccaagatcccacaaacaagttcggttcagtcgttcgtagatgttgatgcttctttgcttgccactgactcctccctaagtaaagattcattctctgcagactcaacagttgatgtattgtttgagcagtcagttcaagagccatacttttaccgttcaaactctgcggaaacagcacctccatccacagcatgctcctcagcgactctcgatctggtatccactttggaagcaaccacaacgttatttgaggaagccgccaactccaaaaaacaaatgattgggcttaaacagcaatccaagatcgaagatctcagtttttcctgcaaactaaatttctcagcatcaatgttccttgaccattcatgcataccgctggtgcgtgggcgtagctttggaggaaatagcagacctcccaagacgccattaattcctgggacgcggtacgttactgcccgtctaagactggagaacagtacaagtgaagatatgtgttttccccactggagcgagcgggaacttagcgactcaagaagaatcattcgtattgagagaagttaccagtccaatgaaattgtagcatcttttttcatagttggatcggccatagatcacccggagactaaaccttcatctgaattagacgtgaaagttttagaagtttcgtgtctaagatgttttatcaacgacaatgaatctgaggaggagcgatttgtcagtgaatatacctcagatgaagcacgagcacaacatgccttaacagaaccgctaacgtcaggcttgaacaaagaaccgaatggccataaccctaaaacggctgggaattcaattggatgcaggtactatataaccagtgttgaagtcatcaagataatagaactactgattggaagctattcaacctcagaccctgtgcaaaggagaaaagaaagaggccgtatgagatcgaacttggtgcagttctggtccaaacacctagtgtcttcctccaaggacactatgaagcgccgttccgtgccaacatgcaacgatgattatctttcggagctggaacatcgtattaacacatatgacgtgagaaaacctcgaaaatttgacaagagcgttaagattctggaatggtccaaacttgggccagctttgcagcgggctatccaaagctattacatggtccgattagataattccagcaataaaactgcagcaacaaatagcagctaa

SEQ ID NO: 40 - DNA, 54 bp, Artificial
  Primer
caatcaattgaacaactatcaaaacacaatgcaattgtttttagcaactctagg

SEQ ID NO: 41 - DNA, 53 bp, Artificial
  Primer
ccgcttttctttttttttcttcttattgtcttagctgctatttgttgctgcag

SEQ ID NO: 42 - DNA, 1596 bp, Komagataella pastoris
atgcagttttacttagctactcctaccactcctttcgttgataacttgacaataccctctggtagcggggcttatagcaaatccaacggcaacaatagcagcagcttcggtcatagttacaacggaaaagaagagctcactgatgaccttctgactgcagatttcgtcgacccaccaatcgacgacgcaatattgtactcgatgctatctcctttcatgaatgctactaccgaacaaccacaagaattccagaatgaacagcagcatcagaacctttccggttgctaccaggagctgctggttcaaacagaggaacaacaacaactggatttgttgcctaatatgcaccccgagaatattttcaattctgagtcttctatgttcgtgtactccagtatcccgcaaagaaattctgttcactcgctgatcagtgtcaacacctcttctcttgcccccgattctgctgaggggggaggcttatcgtttgtggacaccataggcccatcatttgaccattctgttgaaacctcactctcttacggaatgaactctacggactcaactctaccgtcctcagcacgctcctcggtaggacttgacctactacccaccgtggcagctcctgcgtcatccgtttcctcattggaaacggtcatcaactccaagagacaaaagctgcaattcagacagcacaacaggattgaagaactcaattctccacacggtatcaaagcacaggcaccggtgttgcttaactactcaagtcttccgctggttcgtggacttagctctggtgggaacaacagcagacctcccaaagagccattaattcctggagcacagtacattactgcccttctgagactagagaacagctcctgcgaggacttatgtctaccagaatggaacgaggacgaactgaaggactgcagaaggatcattcgcatagagagaaaacgccactccaacgagatagtggcgtcattttctatagtcggttctgccacgcagaacccacaaatcaagccagcatccgactctgacgtgcaagtgttggaagtctcatgtttacaatgcctcgtcaaccataacgaaactgacgaggggaaacaaggaagctccccaaacgaaatacagcatagctctgcagagccaccgaaatcgaacctcaacaggacattgagtagtagtagccacagagcagctgggagctccgtgggataccagtactacatcaccagtgtggaagtcatcaagattatagaactgctagtcggaagccatttaatcgaagactcgcagcaaaagagaaaagaaagaggccgcgtgagatcaaacttggtgcccttctggtcaaaacatcccgtgtcctcgtctaagaagaccttgaggccccgttctggaccagcttgcaacgatgactaccttgcagagttggcccatcgtatcatgacatacgagatacgcaggccgcgaatattcgacaagaacgtacgaattctggaatgggccaagcttgccccggctttgcagcgggctcttcaaagctactatgtggcggtgcccctagatgaatccgagaataaacttgtataa

SEQ ID NO: 43 - DNA, 57 bp, Artificial
  Primer
caatcaattgaacaactatcaaaacacaatgcagttttacttagctactcctaccac

SEQ ID NO: 44 - DNA, 57 bp, Artificial
  Primer
ccgcttttctttttttttcttcttattgtcttatacaagtttattctcggattcatc

SEQ ID NO: 45 - DNA, 1353 bp, Komagataella pastoris
atgttaacagcaggttcagttcagcggctacaaatatctatgtttcaaacacaggaacaggagcaggtggacctttcgtcctctatacataccgagaacattttcaattcagaatcgtctggtcccgcatatccgaacatcccacaaattagttctgttcactcgtttataggtgccaaaagctctcttcttgctgatgctgctccgaatcaatgtttatcatttgtggactcagcaaaagttacatcgtttgaccaatcattccatggttcaccatcttataagctaaattctgcaaaactagcagtttcatcgtctgagtacttgtcagtagaatcatccacagaggtagactccacaatttcattggaagcacaagttgaccgcaaaagacaaaagattcaacccgaacgacacagcaaaaccgacaatctgaacctcttttctgacttcgatctattggcaccagcgtttcttacctactacttgccattgtttcgtggatgtagctctggaggcaacaacaaacccccgaaaaagcgatcaattactggaacacagtacgttgccgccagtttgagactggaaagcagtaccattgaggacatttgcttaccgaaatggaacgagaacgagctcaaggacaatagaagaatcatccgcatagagagaagacaacagggaaatgaaattgcagcttccttttcaatagttgggtctgctttagagaacccacacaccaaacctacactggatcctgacgtggaagtattagaagtctcgtgcttacggtgtctagtcaattacgacgagattgatgacaaacaaacagtgcatggaagccttttacacgaagttcagtctgaattcaccgaaatacggaaacaagaatctaccgggaaacgtagcagttatgcctgtacgtcgaatgggacctccatcgaatgccagtactacgttactagtgtcgaagtcatcaagataattgagttactggtgggaagccatttaatccaagatcgcaagcagagaagaaaagaaaggggctgcataaggtcaaatttgatgcccttctggtccaaacatctcatctcgtcaaccacaaacaccatgaagcgccgttcagtgacagcatctaacgagaattatattgcagagctggcccatcgtatcaagacatatgaagttcggagacctcgaaaggtcaataaggatctcagaattttggaatggtcaaagcttggaccagctctgcggcgggctctccacagctattacgtagcagtgccaattgataaattcaaaaatatagccaagccgacaaacagtgagctaatgtttaatatccaataa

SEQ ID NO: 46 - DNA, 50 bp, Artificial
  Primer
caatcaattgaacaactatcaaaacacaatgttaacagcaggttcagttc

SEQ ID NO: 47 - DNA, 62 bp, Artificial
  Primer
ccgcttttctttttttttcttcttattgtcttattggatattaaacattagctcactgtttg

SEQ ID NO: 48 - DNA, 1245 bp, Komagataella pastoris
atgtttcaaccactcgagcaagaacaacaactggatttgttgtttgccatgcctccggagaacatttttaactctgactcttctgcacttaccttttccaatacttttcaaaccagttcagttcactcggtcacaggtgttgacagcccttcacttgttaatattaatatcccaaaatcagaatcgttctttatggactcaatgacgggtgcatcttttggacaaacttttcaagacctattcccagcgtttcctttgctaaaacttcctaccatggtagcacctccaacttcgttgaagatggtcatggaccgtaaacggcgacagtcccaaataacacagcagaataccactgaaaatctcaattcctttcatggcatgaatatgccggtaccagcgttccttgaatactcggccttcccgctggttcgtggcttttgctctggaggaaaaataaagccgcctaagaaccaactagttcctggaactgagtacgtcaatgtctgcttggagttgctcaacgtcaactatgaaggcatttgtttacctaaatggaatgagaaagaacttgaggaaagtagaaggattgttcgagtagagaaaagacgtcagtataataaaattgaggctgccttttcaatagttgattctgttcaagaaaatccacaaaccaatattgcagttgaccctaacgttgatgtgctggagttttcgtgtttattgtgtccaatcaaagaacacaactcagaagtagcgcaaaccgtccctacaagctcttcaagtgaagaacacaagtacagcgagctagcaggacacgactttgaagaaaatttgagctgtggtagtgataaagaaattgagacttctgttagatttgaatactacatcaccagtgttgaagtcattaggattatagagtttttaacggaaaatcatctaatccaagatatccaggcgaaaagaagagaaagaagctgtctaagatcaaatttagtgcgattctggtccaaagatatagttccatcgtccacaagtaccatgagcaagggttctaagccatctcgggaaaacgattgtattgaagagttggcccaacgtatcagggcttataaggtgcggaaacctctaatagtcgataaaagcctaagaattctggaatggtccaaacttggaccagctttggagcgggctcttcaaagttattacgtagcgttaccattggataaaattaaaacaaagttatga

SEQ ID NO: 49 - DNA, 56 bp, Artificial
  Primer
caatcaattgaacaactatcaaaacacaatgtttcaaccactcgagcaagaacaac

SEQ ID NO: 50 - DNA, 70 bp, Artificial
  Primer
ccgcttttctttttttttcttcttattgtctcataactttgttttaattttatccaatggtaacgctacg

SEQ ID NO: 51 - DNA, 1596 bp, Komagataella pastoris
atgcggttgtatttagcaactgtaggaactccattcatcggtgcaccgccagtgattgctagtaatgaagaccatagcactgctcacaataacaatagcaacgactttaattatgaatgctataataaagaaaagctctctggggatctcttggaagctgacctttttgatcctccaattgatgatacaatgcactgctccatgttttcgtcatcaacaactgctgacactgagcagctacaaaaaccaaagcatgaatcgcaacacaagaatcccttcgattgttgtcaggaactcaagcttaaaatggccgatcagcaagagttgcatttgttgccggagaaaattaccaagaatatttttaattcagagtcgcctgtgttcatatgcaccaagattccccaaacaagtccggtccagtcgttcgtagatgttaatgcttctttacttgccactaactccgccctgactacaaattcattctctgtagacacaacagttgatatattgtttgagcaaacagttcaagatccatactcttaccgtttaaactctaaggaaacagcacctccatccacagctagcttctttgggactctcgatctgggacccaccgtggaagcatccacaacgttactggaggcagccgtcaactctaaaagacgaatagttgaacttagacagcaaaacagaatcgaggatctcaatttttcaacatcaatgttccttgaccattcacgcataccgttggtgcgtgggcgtagctttggaggaaatagcagcagacctcccaagacgccattaattcctgggacgcggtacgttactgcccgtttaagactggaaaacagcacaagtgaggatatatgtttgccccattggaacaaagcggaactcaaagactctagaagaataatccgtattgaaagaagataccagtccaatgagattgtagcaaccttttccatagttggatcggccatagagcacccaaagactaagcctgcattcgatcctgacgtcaaggttttggaagtctcgtgtctaaaatgtcccaccaacgaaaatgaacctaacgaggagcgatttgtcagtgaatgtacgtcagatgaagcacgagcacaacatgccttaacagaacccctaatgtcaggcttcaacagagaaccgaatggccgtaaccctaaaacggctgggaattcaattggatgcaggtactatattaccagtgtcgaagtcatcaagataatagaactactgattggaagctattcaacctcagaccccgtgcaaaggagaaaagagagaggccgtatacgatctaacttggtgcagttctggtccaaacacctagtcttttcctcgaagaactctatgaagcgccgttccgtgccaacaggcaacgatgcttatcttacggagctggagcatcgtattaacacttatgacgtgagaaaacctcgactatttgacaagagtgtcaagattctggaatggtccaaacttgggccagctttgcagcgggctatccaaagctattacatggttcggctagataattccagcgacataactgcaacaacaaatagcagctaa

SEQ ID NO: 52 - DNA, 54 bp, Artificial; identical to SEQ ID NO: 31
  Primer

SEQ ID NO: 53 - DNA, 53 bp, Artificial; identical to SEQ ID NO: 35
  Primer

SEQ ID NO: 54 - DNA, 1542 bp, Komagataella pastoris
atgcagttttacttagctactcctaccactcctttcgttgataacttgacaataccctctggtagcggggcttatagcaaatccaacggcaacaatagcagcagcttcggtcatagttacaacggaaaagaagagctcactgatgaccttctgactgcagatttcgtcgacccaccaatcgacgacgcaatattgtactcgatgctatctcctttcatgaatgctactaccgaacaaccacaagaattccagaatgaacagcagcatcagaacctttccggttgctaccaggagctgctggttcaaacagaggaacaacaacaactggatttgttgcctaatatgcaccccgagaatattttcaattctgagtcttctatgttcgtgtactccagtatcccgcaaagaaattctgttcactcgctgatcagtgtcaacacctcttctcttgcccccgattctgctgaggggggaggcttatcgtttgtggacaccataggcccatcatttgaccattctgttgaaacctcactctcttacggaatgaactctacggactcaactctaccgtcctcagcacgctcctcggtaggacttgacctactacccaccgtggcagctcctgcgtcatccgtttcctcattggaaacggtcatcaactccaagagacaaaagctgcaattcagacagcacaacaggattgaagaactcaattctccacacggtatcaaagcacaggcaccggtgttgcttaactactcaagtcttccgctggttcgtggacttagctctggtgggaacaacagcagacctcccaaagagccattaattcctggagcacagtacattactgcccttctgagactagagaacagctcctgcgaggacttatgtctaccagaatggaacgaggacgaactgaaggactgcagaaggatcattcgcatagagagaaaacgccactccaacgagatagtggccgactctgacgtgcaagtgttggaagtctcatgtttacaatgcctcgtcaaccataacgaaactgacgaggggaaacaaggaagctccccaaacgaaatacagcatagctctgcagagccaccgaaatcgaacctcaacaggacattgagtagtagtagccacagagcagctgggagctccgtgggataccagtactacatcaccagtgtggaagtcatcaagattatagaactgctagtcggaagccatttaatcgaagactcgcagcaaaagagaaaagaaagaggccgcgtgagatcaaacttggtgcccttctggtcaaaacatcccgtgtcctcgtctaagaagaccttgaggccccgttctggaccagcttgcaacgatgactaccttgcagagttggcccatcgtatcatgacatacgagatacgcaggccgcgaatattcgacaagaacgtacgaattctggaatgggccaagcttgccccggctttgcagcgggctcttcaaagctactatgtggcggtgcccctagatgaatccgagaataaacttgtataa

SEQ ID NO: 55 - DNA, 57 bp, Artificial; identical to SEQ ID NO: 43
  Primer

SEQ ID NO: 56 - DNA, 57 bp, Artificial; identical to SEQ ID NO: 44
  Primer

SEQ ID NO: 57 - DNA, 1566 bp, Komagataella pastoris
atgttaacagcaggttcagttcagcggctacaaatatctatgtttcaaacacaggaacaggagcaggtggacctttcgtcctctatacataccgagaacattttcaattcagaatcgtctggtcccgcatatccgaacatcccacaaattagttctgttcactcgtttataggtgccaaaagctctcttcttgctgatgctgctccgaatcaatgtttatcatttgtggactcagcaaaagttacatcgtttgaccaatcattccatggttcaccatcttataagctaaattctgcaaaactagcagtttcatcgtctgagtacttgtcagtagaatcatccacagaggtagactccacaatttcattggaagcacaagttgaccgcaaaagacaaaagattcaacccgaacgacacagcaaaaccgacaatctgaacctcttttctgacttcgatctattggcaccagcgtttcttacctactacttgccattgtttcgtggatgtagctctggaggcaacaacaaacccccgaaaaagcgatcaattactggaacacagtacgttgccgccagtttgagactggaaagcagtaccattgaggacatttgcttaccgaaatggaacgagaacgagctcaaggacaatagaagaatcatccgcatagagagaagacaacagggaaatgaaattgcagcttccttttcaatagttgggtctgctttagagaacccacacaccaaacctacactggatcctgacgtggaagtattagaagtctcgtgcttacggtgtctagtcaattacgacgagattgatgacaaacaaacagtgcatggaagccttttacacgaagttcagtctgaattcaccgaaatacggaaacaagaatctaccgggaaacgtagcagttatgcctgtacgtcgaatgggacctccatcgaatgccagtactacgttactagtgtcgaagtcatcaagataattgagttactggtgggaagccatttaatccaagatcgcaagcagagaagaaaagaaaggggctgcataaggtcaaatttgatgcccttctggtccaaacatctcatctcgtcaaccacaaacaccatgaagcgccgttcagtgacagcatctaacgagaattatattgcagagctggcccatcgtatcaagacatatgaagttcggagacctcgaaaggtcaataaggatctcagaattttggaatggtcaaagcttggaccagctctgcggcgggctctccacagctattacgtagcagtgccaattgataaattcaaaaatatagccaagccgacaaacactagcctaggcaccataaagatggcaatagctggtagaagtgaggttttggaaacagtctttatagcaagacaaagtccggttgaaccagatgcttgctcatcgattgctaacgtcagaagggaattgtgcgacgcttgcactggcacacaggccaatattgtgactgaaaagattcttctgactaccatagccgcggacgccggatctacaattgtggtgccagtgtgcgttccttaa

SEQ ID NO: 58 - DNA, 50 bp, Artificial; identical to SEQ ID NO: 46
  Primer

SEQ ID NO: 59 - DNA, 52 bp, Artificial
  Primer
ccgcttttctttttttttcttcttattgtcttaaggaacgcacactggcacc

SEQ ID NO: 60 - DNA, 1575 bp, Komagataella pastoris
atgcggttgtctttagcaacacgagaaacaccgtctaccggtacatcgccggtgtttgctggtgacgaagatcatagcagtgccaacaatagcagtagcaacactttcaactatgactactatataaaagaagaactctctggaaatctcctggaaacagccttttttgaatctccgattaatgatacaatgctctgctccatgttatcgtatccaaaaattgctggtactgagaaaatacaagaaccagaccatgaactgcagcaacacaggaatcccttcggttgctgccaggaactcaaagctgaattggccgaacagcaagagctgtatctgctgccaaataaacttaccaagaatatttttaattcagggtctcctgtgttcatatacaccaagatcccacaaacaagctcggttcagtcatttgtggatgttagcaattctttatttgccactgactccgccttgcctacaagttcatttcgtgtagactcaacagtcgacatatcgtttgagcactcaattcaagattcatactgttaccgttcaaactctgcagaaatagcacctccatccacagcactctcttcagggactcacgacctggtatccaccgtggaagcatccacgacgttactggaggcagccgtcaactctaaaagacgaatgcttgaacttagacagcaaaacagaatcgaggatctcagtttttcaacatcaatattccttgaccattcacgcctaccgttggttcgtgggcgtagctttggaggaaatagcagtagacctcccaagacgccattgattcctgggacgcggtacgttactgcccgtttaagactggaaaacagcacaagtgaggatatgtgtttgccccagtggaacaaggcggaactcaaagactctagaagaataatccgtattgaaagaagataccagtctgatgaaattgtcgcttccttttccatagttggatcggccatagagtacccaaagactaagcctgcattcgatcctgacgtcaaggttttggaagtctcgtgtctaaaatgtcccaccaacgaaaatgaacctaacgaggagcgatttgtcagtgaatgtacgtcagatgaagcacgaacacaacatgccttaacagaacccctaacgtcaggcttcaacagagaaccgaatggccataaccctaaaacggctgggaattcaattggatgcaggtactatattaccagtgtcgaagtcatcaagataatagaactactgattggaagctattcaacctcagaccccgtgcaaaggagaaaagagagaggccgtatacgatctaacttggtgcagttctggtccaaacacctagtcttttcctcgaagaactctatgaagcgccgttccgtgccaacaggcaacgatgcttatcttacggagctggagcatcgtattaacacttatgatgtgagaaaacctcgactatttgataagagtgtcaagattctggaatggtctaaacttggacctgctttgcgtcgggctatccaaagctattacatggtacgactagacgaatccagcaactaa

SEQ ID NO: 61 - DNA, 51 bp, Artificial; identical to SEQ ID NO: 34
  Primer

SEQ ID NO: 62 - DNA, 54 bp, Artificial; identical to SEQ ID NO: 32
  Primer

SEQ ID NO: 63 - DNA, 28 bp, Artificial
  Primer
tgtgttttgatagttgttcaattgattg

SEQ ID NO: 64 - DNA, 30 bp, Artificial
  Primer
gacaataagaagaaaaaaaaagaaaagcgg

SEQ ID NO: 65 - DNA, 1107 bp, Artificial
  Synthetic
atgcttgctgagattggggactatttcaggacaaatagtagcaatggattcagttctagctgctataacaaagagaagctttcaggagacctcttggagtccgattttgtgaattcatcattcgatgacacaacactccactctatgctatcttcttcaataaatgctgatccggagcaatcccagaaaccacaacaaagacacaagaatcaatccagttgccatcaggaactcaagattgaaacagaagatcagcaagaaccgtatctattgcctgatatgcttaccaaaaacatattcaactcacagtcttctatgtttgtctactccagcatcccacaaataagctcagtgcaatcgtttatagacacaaacaccttttccgctgttgatggtggtgccgccccggaaggaaattcattgtctgtggactcaactttaggtctatcttctgaccaatcatttgagaagctatactcttacgaccaaaactttgagaaagaaccacaatcgccagcatgtcccttagtaaaatgtgacccagtaagcatcatgaaaacatccacaaactcattggagacagctgtcaacaccaaaagacgaatgtttcagcttagaaaccaaaatgaaattgaaagtctcaatgtttctagtgacatcgttgttgcgacatcaaaatttctcaatcattcatggatcccgctgatccgtggacgtaatttcggaggaagcaactgcaaaccgcccaagaaaccattatttcctggagcgcgctatgttactgcccgtctacgattggaatacagtagctgtaaagatatgtgtctaccggaatggaacgagtatgaactcgaagacagtagaagaatcattcgtattgagagactgttcgactccaatgaaatcgtagcctccttttccattgtcgggtctgctgtagagaacccggaaactaggcctgtattcaatcctaatgtcaaagtgttggaagtttcgtgtctaagatgtcttaccaacaacaacgagtctgacgaggagaactgtgtcaacaaagatttggatggccgtaaccgcgatatggttaaggactcatttggatgcaagtaa

SEQ ID NO: 66 - DNA, 58 bp, Artificial
  Primer
ccgcttttctttttttttcttcttattgtcttacttgcatccaaatgagtccttaacc

SEQ ID NO: 67 - DNA, 1203 bp, Artificial
  Synthetic
atgaattcatcttcaactcgtggcctgcaagctatatcactcacaccaactagagttttattcactttgagcactctgttcgccgatacgtcaaaaatgtctgctgagaatggggactatttcaggacaaacagtagcaatgaattcagcaataactgctttaacaaagaaaagctctcggaggacctcttggaggccagttttgtcgattccccaatcgaagacacaataccccactttatgctatcttcttcaataaatgctgattcggaacagcctcaggaaccacagcatcgacacaagaatcaatccagttgccatcgggaactcaagatcgaaacaaacgatcagcaagaaccgtatctgttgcctgatatgcttaccaaaaacatattcaactcacagtcttctatgtttgtctattccagcatcccacaaacaagctcagtgcaatcgtttatagacacaaacattttttccgctgttgatggtggtgccgccccagaagaaaattcattgtctgtggactcaaccttaggtccatcttctgaccaatcatttgaaaagctatactcttacgaccaaaattttgagaaagaaccacaatcgccagcatgtcccttagtaaaatgtgacctagtaggcatcatgaaagcatccacaaactcattggaggtagctgtcaacaccaaaagacaaatgtttcagcttagacaccaaaatgaaattgagagtctctatgtttctagtgacatcgttattgcgacatcaaattttcttgaccattcatggattccgctgatccgtggacgtaatttcggaggaagcaactgcaaaccgcccaagaaaccattatttcctggagcgcgctatgttactgcccgtctacgattggaatacagtagctgtgaagatatgtgtttaccggaatggaacgagtatgaactcaaagacagtagaagaatcattcgtcttgagaggcggttcgactccaatgaaatcgtggcctccttttccattgtcgggtctgctgtagagaaccctgaaactaggcctgcattcaatcctaacgtcaaagtgttggaagtttcgtgtctaaggtgtcttaccaacaacaacgaatctgacgaggagaactgtgtcaacaaagatttggatggccgtgtccacaatatggttaaagactcaattggatgcaagtaa

SEQ ID NO: 68 - DNA, 58 bp, Artificial
  Primer
ccgcttttctttttttttcttcttattgtcttacttgcatccaattgagtctttaacc

SEQ ID NO: 69 - DNA, 1218 bp, Artificial
  Synthetic
atgcggttgtatttagcaactgtaggaactccattcatcggtgcaccgccagtgattgctagtaatgaagaccatagcactgctcacaataacaatagcaacgactttaattatgaatgctataataaagaaaagctctctggggatctcttggaagctgacctttttgatcctccaattgatgatacaatgcactgctccatgttttcgtcatcaacaactgctgacactgagcagctacaaaaaccaaagcatgaatcgcaacacaagaatcccttcgattgttgtcaggaactcaagcttaaaatggccgatcagcaagagttgcatttgttgccggagaaaattaccaagaatatttttaattcagagtcgcctgtgttcatatgcaccaagattccccaaacaagtccggtccagtcgttcgtagatgttaatgcttctttacttgccactaactccgccctgactacaaattcattctctgtagacacaacagttgatatattgtttgagcaaacagttcaagatccatactcttaccgtttaaactctaaggaaacagcacctccatccacagctagcttctttgggactctcgatctgggacccaccgtggaagcatccacaacgttactggaggcagccgtcaactctaaaagacgaatagttgaacttagacagcaaaacagaatcgaggatctcaatttttcaacatcaatgttccttgaccattcacgcataccgttggtgcgtgggcgtagctttggaggaaatagcagcagacctcccaagacgccattaattcctgggacgcggtacgttactgcccgtttaagactggaaaacagcacaagtgaggatatatgtttgccccattggaacaaagcggaactcaaagactctagaagaataatccgtattgaaagaagataccagtccaatgagattgtagcaaccttttccatagttggatcggccatagagcacccaaagactaagcctgcattcgatcctgacgtcaaggttttggaagtctcgtgtctaaaatgtcccaccaacgaaaatgaacctaacgaggagcgatttgtcagtgaatgtacgtcagatgaagcacgagcacaacatgccttaacagaacccctaatgtcaggcttcaacagagaaccgaatggccgtaaccctaaaacggctgggaattcaattggatgcaggtaa

SEQ ID NO: 70 - DNA, 54 bp, Artificial
  Primer
ccgcttttctttttttttcttcttattgtcttacctgcatccaattgaattccc

SEQ ID NO: 71 - DNA, 1221 bp, Artificial
  Synthetic
atgcggttgtctttagcaacacgagaaacaccgtctaccggtacatcgccggtgtttgctggtgacgaagatcatagcagtgccaacaatagcagtagcaacactttcaactatgactactatataaaagaagaactctctggaaatctcctggaaacagccttttttgaatctccgattaatgatacaatgctctgctccatgttatcgtatccaaaaattgctggtactgagaaaatacaagaaccagaccatgaactgcagcaacacaggaatcccttcggttgctgccaggaactcaaagctgaattggccgaacagcaagagctgtatctgctgccaaataaacttaccaagaatatttttaattcagggtctcctgtgttcatatacaccaagatcccacaaacaagctcggttcagtcatttgtggatgttagcaattctttatttgccactgactccgccttgcctacaagttcatttcgtgtagactcaacagtcgacatatcgtttgagcactcaattcaagattcatactgttaccgttcaaactctgcagaaatagcacctccatccacagcactctcttcagggactcacgacctggtatccaccgtggaagcatccacgacgttactggaggcagccgtcaactctaaaagacgaatgcttgaacttagacagcaaaacagaatcgaggatctcagtttttcaacatcaatattccttgaccattcacgcctaccgttggttcgtgggcgtagctttggaggaaatagcagtagacctcccaagacgccattgattcctgggacgcggtacgttactgcccgtttaagactggaaaacagcacaagtgaggatatgtgtttgccccagtggaacaaggcggaactcaaagactctagaagaataatccgtattgaaagaagataccagtctgatgaaattgtcgcttccttttccatagttggatcggccatagagtacccaaagactaagcctgcattcgatcctgacgtcaaggttttggaagtctcgtgtctaaaatgtcccaccaacgaaaatgaacctaacgaggagcgatttgtcagtgaatgtacgtcagatgaagcacgaacacaacatgccttaacagaacccctaacgtcaggcttcaacagagaaccgaatggccataaccctaaaacggctgggaattcaattggatgcaggtaa

SEQ ID NO: 72 - DNA, 54 bp, Artificial; identical to SEQ ID NO: 70
  Primer

SEQ ID NO: 73 - DNA, 1170 bp, Artificial
  Synthetic
atgcagctactgtcagcaagttcaagcactccattcgtcgctatgtcgacagtaaacgctgggaacgagtgttatcttaggaccaacaatagtaacagcaacggtatcagcgataaccgcttcagaaaaaaaaagctttctgaggatttgttgaaagccgattttgtcgaagacacagccggtaacccaatgatttaccccatggttttatctgctccaacatatataaaaactgaacatctccaagaattggagcataaacagcaaggatcaaatctgttgtctgaaatgtttaccaaaaatattttcaattcacattctgcaatgtttgtttactctgaaatccctcaaataagttcggttcagtcattcattggttctgacacctctgtccttgttgctgatgcctcttcagaaaaaaattcattgtttgtgtactcaaatactggtatatcttttgatccgtcagatgaggatccatgcccttacgaggctaattttgatccaggacaactattctcagcggtatccgcagtagaagacggttctatggcaacattgacagctcccactatgtcattagaaggtgtcgttaattccaaaaaaggaatatgtcaacttagacagcataacagaatcgagggatttgattcttcccatgaattcaaagtcttgggatcaatgttccttaaccattcgtacgtaccgctggttcgtggacgtagtttcggaggaaacaattgtagacctcccaaagagccattaattcctggaacccggtacgttgctgcccatctaagattggaaaacaatacctgcgaggatatgtgtctcccagaatggaacaagcatgagcttgaagatagtagaagaatcattcgtatagagagaagataccagtcgaatgaaattgtggcctctttttctatagttgggtctgctaaagagaatccacaaacaaagccttcaagcgatcctgacgtgaaagttttggaagtctcgtgtctgaaatgtttcatcaacggcaacgaatctgacgagcaatttgtgactgaatgtcagtcagatgaagcgcattatggcttcacagagttaccaaaacaaaactttagcagaaaatcaaaaggctgcagccacaaaatggcaaataacacgattgcatgccaataa

SEQ ID NO: 74 - DNA, 58 bp, Artificial
  Primer
ccgcttttctttttttttcttcttattgtcttattggcatgcaatcgtgttatttgcc

SEQ ID NO: 75 - DNA, 1236 bp, Artificial
  Synthetic
atgcaattgtttttagcaactctaggaattccgttcaccggcacattaccagccattgctggtaacgaggatcatagcagtgccaacaacaacaatggcaacggtttcagttatgactggcatggaaatgatacgctttttgcggaactcttggaaactgacttttttgatcatccgattgatgacgcaatgctctgctccttgttatcccctccaagagctgctgagactgagcaacagcaagatacagaatgggagcaacaaaaacacacaaatctctttggttgctgccagaaacacaaagttgaaatggccaaacagcaagaactgtatttgctgccagataagcttaccaagaatattttcaattcagagtcgcctgtgttcatatgcaccaagatcccacaaacaagttcggttcagtcgttcgtagatgttgatgcttctttgcttgccactgactcctccctaagtaaagattcattctctgcagactcaacagttgatgtattgtttgagcagtcagttcaagagccatacttttaccgttcaaactctgcggaaacagcacctccatccacagcatgctcctcagcgactctcgatctggtatccactttggaagcaaccacaacgttatttgaggaagccgccaactccaaaaaacaaatgattgggcttaaacagcaatccaagatcgaagatctcagtttttcctgcaaactaaatttctcagcatcaatgttccttgaccattcatgcataccgctggtgcgtgggcgtagctttggaggaaatagcagacctcccaagacgccattaattcctgggacgcggtacgttactgcccgtctaagactggagaacagtacaagtgaagatatgtgttttccccactggagcgagcgggaacttagcgactcaagaagaatcattcgtattgagagaagttaccagtccaatgaaattgtagcatcttttttcatagttggatcggccatagatcacccggagactaaaccttcatctgaattagacgtgaaagttttagaagtttcgtgtctaagatgttttatcaacgacaatgaatctgaggaggagcgatttgtcagtgaatatacctcagatgaagcacgagcacaacatgccttaacagaaccgctaacgtcaggcttgaacaaagaaccgaatggccataaccctaaaacggctgggaattcaattggatgcaggtaa

SEQ ID NO: 76 - DNA, 54 bp, Artificial; identical to SEQ ID NO: 70
  Primer

SEQ ID NO: 77 - DNA, 1230 bp, Artificial
  Synthetic
atgcagttttacttagctactcctaccactcctttcgttgataacttgacaataccctctggtagcggggcttatagcaaatccaacggcaacaatagcagcagcttcggtcatagttacaacggaaaagaagagctcactgatgaccttctgactgcagatttcgtcgacccaccaatcgacgacgcaatattgtactcgatgctatctcctttcatgaatgctactaccgaacaaccacaagaattccagaatgaacagcagcatcagaacctttccggttgctaccaggagctgctggttcaaacagaggaacaacaacaactggatttgttgcctaatatgcaccccgagaatattttcaattctgagtcttctatgttcgtgtactccagtatcccgcaaagaaattctgttcactcgctgatcagtgtcaacacctcttctcttgcccccgattctgctgaggggggaggcttatcgtttgtggacaccataggcccatcatttgaccattctgttgaaacctcactctcttacggaatgaactctacggactcaactctaccgtcctcagcacgctcctcggtaggacttgacctactacccaccgtggcagctcctgcgtcatccgtttcctcattggaaacggtcatcaactccaagagacaaaagctgcaattcagacagcacaacaggattgaagaactcaattctccacacggtatcaaagcacaggcaccggtgttgcttaactactcaagtcttccgctggttcgtggacttagctctggtgggaacaacagcagacctcccaaagagccattaattcctggagcacagtacattactgcccttctgagactagagaacagctcctgcgaggacttatgtctaccagaatggaacgaggacgaactgaaggactgcagaaggatcattcgcatagagagaaaacgccactccaacgagatagtggcgtcattttctatagtcggttctgccacgcagaacccacaaatcaagccagcatccgactctgacgtgcaagtgttggaagtctcatgtttacaatgcctcgtcaaccataacgaaactgacgaggggaaacaaggaagctccccaaacgaaatacagcatagctctgcagagccaccgaaatcgaacctcaacaggacattgagtagtagtagccacagagcagctgggagctccgtgggataccagtaa

SEQ ID NO: 78 - DNA, 54 bp, Artificial
  Primer
ccgcttttctttttttttcttcttattgtcttactggtatcccacggagctccc

SEQ ID NO: 79 - DNA, 954 bp, Artificial
  Synthetic
atgttaacagcaggttcagttcagcggctacaaatatctatgtttcaaacacaggaacaggagcaggtggacctttcgtcctctatacataccgagaacattttcaattcagaatcgtctggtcccgcatatccgaacatcccacaaattagttctgttcactcgtttataggtgccaaaagctctcttcttgctgatgctgctccgaatcaatgtttatcatttgtggactcagcaaaagttacatcgtttgaccaatcattccatggttcaccatcttataagctaaattctgcaaaactagcagtttcatcgtctgagtacttgtcagtagaatcatccacagaggtagactccacaatttcattggaagcacaagttgaccgcaaaagacaaaagattcaacccgaacgacacagcaaaaccgacaatctgaacctcttttctgacttcgatctattggcaccagcgtttcttacctactacttgccattgtttcgtggatgtagctctggaggcaacaacaaacccccgaaaaagcgatcaattactggaacacagtacgttgccgccagtttgagactggaaagcagtaccattgaggacatttgcttaccgaaatggaacgagaacgagctcaaggacaatagaagaatcatccgcatagagagaagacaacagggaaatgaaattgcagcttccttttcaatagttgggtctgctttagagaacccacacaccaaacctacactggatcctgacgtggaagtattagaagtctcgtgcttacggtgtctagtcaattacgacgagattgatgacaaacaaacagtgcatggaagccttttacacgaagttcagtctgaattcaccgaaatacggaaacaagaatctaccgggaaacgtagcagttatgcctgtacgtcgaatgggacctccatcgaatgccagtaa

SEQ ID NO: 80 - DNA, 54 bp, Artificial
  Primer
ccgcttttctttttttttcttcttattgtcttactggcattcgatggaggtccc

SEQ ID NO: 81 - DNA, 882 bp, Artificial
  Synthetic
atgtttcaaccactcgagcaagaacaacaactggatttgttgtttgccatgcctccggagaacatttttaactctgactcttctgcacttaccttttccaatacttttcaaaccagttcagttcactcggtcacaggtgttgacagcccttcacttgttaatattaatatcccaaaatcagaatcgttctttatggactcaatgacgggtgcatcttttggacaaacttttcaagacctattcccagcgtttcctttgctaaaacttcctaccatggtagcacctccaacttcgttgaagatggtcatggaccgtaaacggcgacagtcccaaataacacagcagaataccactgaaaatctcaattcctttcatggcatgaatatgccggtaccagcgttccttgaatactcggccttcccgctggttcgtggcttttgctctggaggaaaaataaagccgcctaagaaccaactagttcctggaactgagtacgtcaatgtctgcttggagttgctcaacgtcaactatgaaggcatttgtttacctaaatggaatgagaaagaacttgaggaaagtagaaggattgttcgagtagagaaaagacgtcagtataataaaattgaggctgccttttcaatagttgattctgttcaagaaaatccacaaaccaatattgcagttgaccctaacgttgatgtgctggagttttcgtgtttattgtgtccaatcaaagaacacaactcagaagtagcgcaaaccgtccctacaagctcttcaagtgaagaacacaagtacagcgagctagcaggacacgactttgaagaaaatttgagctgtggtagtgataaagaaattgagacttctgttagatttgaataa

SEQ ID NO: 82 - DNA, 60 bp, Artificial
  Primer
ccgcttttctttttttttcttcttattgtcttattcaaatctaacagaagtctcaatttc

SEQ ID NO: 83 - DNA, 1218 bp, Artificial; identical to SEQ ID NO: 69
  Synthetic

SEQ ID NO: 84 - DNA, 54 bp, Artificial; identical to SEQ ID NO: 70
  Primer

SEQ ID NO: 85 - DNA, 1176 bp, Artificial
  Synthetic
atgcagttttacttagctactcctaccactcctttcgttgataacttgacaataccctctggtagcggggcttatagcaaatccaacggcaacaatagcagcagcttcggtcatagttacaacggaaaagaagagctcactgatgaccttctgactgcagatttcgtcgacccaccaatcgacgacgcaatattgtactcgatgctatctcctttcatgaatgctactaccgaacaaccacaagaattccagaatgaacagcagcatcagaacctttccggttgctaccaggagctgctggttcaaacagaggaacaacaacaactggatttgttgcctaatatgcaccccgagaatattttcaattctgagtcttctatgttcgtgtactccagtatcccgcaaagaaattctgttcactcgctgatcagtgtcaacacctcttctcttgcccccgattctgctgaggggggaggcttatcgtttgtggacaccataggcccatcatttgaccattctgttgaaacctcactctcttacggaatgaactctacggactcaactctaccgtcctcagcacgctcctcggtaggacttgacctactacccaccgtggcagctcctgcgtcatccgtttcctcattggaaacggtcatcaactccaagagacaaaagctgcaattcagacagcacaacaggattgaagaactcaattctccacacggtatcaaagcacaggcaccggtgttgcttaactactcaagtcttccgctggttcgtggacttagctctggtgggaacaacagcagacctcccaaagagccattaattcctggagcacagtacattactgcccttctgagactagagaacagctcctgcgaggacttatgtctaccagaatggaacgaggacgaactgaaggactgcagaaggatcattcgcatagagagaaaacgccactccaacgagatagtggccgactctgacgtgcaagtgttggaagtctcatgtttacaatgcctcgtcaaccataacgaaactgacgaggggaaacaaggaagctccccaaacgaaatacagcatagctctgcagagccaccgaaatcgaacctcaacaggacattgagtagtagtagccacagagcagctgggagctccgtgggataccagtaa

SEQ ID NO: 86 - DNA, 54 bp, Artificial; identical to SEQ ID NO: 78
  Primer

SEQ ID NO: 87 - DNA, 954 bp, Artificial; identical to SEQ ID NO: 79
  Synthetic

SEQ ID NO: 88 - DNA, 54 bp, Artificial; identical to SEQ ID NO: 80
  Primer

SEQ ID NO: 89 - DNA, 1221 bp, Artificial; identical to SEQ ID NO: 71
  Synthetic

SEQ ID NO: 90 - DNA, 54 bp, Artificial; identical to SEQ ID NO: 70
  Primer

SEQ ID NO: 91 - DNA, 54 bp, Artificial
  Synthetic
tactatatcaccagtgttgaagtcataaagattatagaattactggtcggaagc

SEQ ID NO: 92 - DNA, 57 bp, Artificial
  Synthetic
attctggaatggtccaaacttgggccagctttgcagcgggctatccaaagctattac

SEQ ID NO: 93 - protein, 18 aa, Artificial
  Synthetic
YYITSVEVIKIIELLVGS

SEQ ID NO: 94 - protein, 19 aa, Artificial
  Synthetic
ILEWSKLGPALQRAIQSYY

SEQ ID NO: 95 - protein, 493 aa, Komagataella pastoris
MLAEIGDYFRTNSSNGFSSSCYNKEKLSGDLLESDFVNSSFDDTTLHSMLSSSINADPEQSQKPQQRHKNQSSCHQELKIETEDQQEPYLLPDMLTKNIFNSQSSMFVYSSIPQISSVQSFIDTNTFSAVDGGAAPEGNSLSVDSTLGLSSDQSFEKLYSYDQNFEKEPQSPACPLVKCDPVSIMKTSTNSLETAVNTKRRMFQLRNQNEIESLNVSSDIVVATSKFLNHSWIPLIRGRNFGGSNCKPPKKPLFPGARYVTARLRLEYSSCKDMCLPEWNEYELEDSRRIIRIERLFDSNEIVASFSIVGSAVENPETRPVFNPNVKVLEVSCLRCLTNNNESDEENCVNKDLDGRNRDMVKDSFGCKYYITSVEVIKIIELLVGSSSISDPHQMRKERGRVRSNLAQFWSKRLVSSSRKTMKQGFLPTCNDDYFAELAHRINTYDVRKPRLFDKCIKILEWSKLKPALQRAMQSYYMVQLDESSNKIATANN

SEQ ID NO: 96 - protein, 518 aa, Komagataella pastoris
MNSSSTRGLQAISLTPTRVLFTLSTLFADTSKMSAENGDYFRTNSSNEFSNNCFNKEKLSEDLLEASFVDSPIEDTIPHFMLSSSINADSEQPQEPQHRHKNQSSCHRELKIETNDQQEPYLLPDMLTKNIFNSQSSMFVYSSIPQTSSVQSFIDTNIFSAVDGGAAPEENSLSVDSTLGPSSDQSFEKLYSYDQNFEKEPQSPACPLVKCDLVGIMKASTNSLEVAVNTKRQMFQLRHQNEIESLYVSSDIVIATSNFLDHSWIPLIRGRNFGGSNCKPPKKPLFPGARYVTARLRLEYSSCEDMCLPEWNEYELKDSRRIIRLERRFDSNEIVASFSIVGSAVENPETRPAFNPNVKVLEVSCLRCLTNNNESDEENCVNKDLDGRVHNMVKDSIGCKYYITSVEVIKIIELLVGSSSISDPQQMRKERGRVRSNLAQFWSKHLVSSSRKTRKQRFLPTCNDDYFAELVHRINTYDVRKPRLFDKSVKILEWPNLGPALQRAIQSYYMVRLEESSN

SEQ ID NO: 97 - protein, 523 aa, Komagataella pastoris
MRLYLATVGTPFIGAPPVIASNEDHSTAHNNNSNDFNYECYNKEKLSGDLLEADLFDPPIDDTMHCSMFSSSTTADTEQLQKPKHESQHKNPFDCCQELKLKMADQQELHLLPEKITKNIFNSESPVFICTKIPQTSPVQSFVDVNASLLATNSALTTNSFSVDTTVDILFEQTVQDPYSYRLNSKETAPPSTASFFGTLDLGPTVEASTTLLEAAVNSKRRIVELRQQNRIEDLNFSTSMFLDHSRIPLVRGRSFGGNSSRPPKTPLIPGTRYVTARLRLENSTSEDICLPHWNKAELKDSRRIIRIERRYQSNEIVATFSIVGSAIEHPKTKPAFDPDVKVLEVSCLKCPTNENEPNEERFVSECTSDEARAQHALTEPLMSGFNREPNGRNPKTAGNSIGCRYYITSVEVIKIIELLIGSYSTSDPVQRRKERGRIRSNLVQFWSKHLVFSSKNSMKRRSVPTGNDAYLTELEHRINTYDVRKPRLFDKSVKILEWSKLGPALRRAIQSYYMVRLDESSN

SEQ ID NO: 98 - protein, 532 aa, Komagataella pastoris
MRLSLATRETPSTGTSPVFAGDEDHSSANNSSSNTFNYDYYIKEELSGNLLETAFFESPINDTMLCSMLSYPKIAGTEKIQEPDHELQQHRNPFGCCQELKAELAEQQELYLLPNKLTKNIFNSGSPVFIYTKIPQTSSVQSFVDVSNSLFATDSALPTSSFRVDSTVDISFEHSIQDSYCYRSNSAEIAPPSTALSSGTHDLVSTVEASTTLLEAAVNSKRRMLELRQQNRIEDLSFSTSIFLDHSRLPLVRGRSFGGNSSRPPKTPLIPGTRYVTARLRLENSTSEDMCLPQWNKAELKDSRRIIRIERRYQSDEIVASFSIVGSAIEYPKTKPAFDPDVKVLEVSCLKCPTNENEPNEERFVSECTSDEARTQHALTEPLTSGFNREPNGHNPKTAGNSIGCRYYITSVEVIKIIELLIGSYSTSDPVQRRKERGRIRSNLVQFWSKHLVFSSKNSMKRRSVPTGNDAYLTELEHRINTYDVRKPRLFDKSVKILEWSKLGPALQRAIQSYYMVRLDNSSDITATTNSS

SEQ ID NO: 99 - protein, 507 aa, Komagataella pastoris
MQLLSASSSTPFVAMSTVNAGNECYLRTNNSNSNGISDNRFRKKKLSEDLLKADFVEDTAGNPMIYPMVLSAPTYIKTEHLQELEHKQQGSNLLSEMFTKNIFNSHSAMFVYSEIPQISSVQSFIGSDTSVLVADASSEKNSLFVYSNTGISFDPSDEDPCPYEANFDPGQLFSAVSAVEDGSMATLTAPTMSLEGVVNSKKGICQLRQHNRIEGFDSSHEFKVLGSMFLNHSYVPLVRGRSFGGNNCRPPKEPLIPGTRYVAAHLRLENNTCEDMCLPEWNKHELEDSRRIIRIERRYQSNEIVASFSIVGSAKENPQTKPSSDPDVKVLEVSCLKCFINGNESDEQFVTECQSDEAHYGFTELPKQNFSRKSKGCSHKMANNTIACQYYITSVEVIKIIELLVGGYTVSDPRQRRKERGRVRSNLAQFWSKHLVSSSKKTMKRPAMLACNEDYLAELEQRINTYDVRKPRLFDKSVKILEWPNLGPALQRAIQSYYMVQLEESSN

SEQ ID NO: 100 - protein, 537 aa, Komagataella pastoris
MQLFLATLGIPFTGTLPAIAGNEDHSSANNNNGNGFSYDWHGNDTLFAELLETDFFDHPIDDAMLCSLLSPPRAAETEQQQDTEWEQQKHTNLFGCCQKHKVEMAKQQELYLLPDKLTKNIFNSESPVFICTKIPQTSSVQSFVDVDASLLATDSSLSKDSFSADSTVDVLFEQSVQEPYFYRSNSAETAPPSTACSSATLDLVSTLEATTTLFEEAANSKKQMIGLKQQSKIEDLSFSCKLNFSASMFLDHSCIPLVRGRSFGGNSRPPKTPLIPGTRYVTARLRLENSTSEDMCFPHWSERELSDSRRIIRIERSYQSNEIVASFFIVGSAIDHPETKPSSELDVKVLEVSCLRCFINDNESEEERFVSEYTSDEARAQHALTEPLTSGLNKEPNGHNPKTAGNSIGCRYYITSVEVIKIIELLIGSYSTSDPVQRRKERGRMRSNLVQFWSKHLVSSSKDTMKRRSVPTCNDDYLSELEHRINTYDVRKPRKFDKSVKILEWSKLGPALQRAIQSYYMVRLDNSSNKTAATNSS

SEQ ID NO: 101 - protein, 531 aa, Komagataella pastoris
MQFYLATPTTPFVDNLTIPSGSGAYSKSNGNNSSSFGHSYNGKEELTDDLLTADFVDPPIDDAILYSMLSPFMNATTEQPQEFQNEQQHQNLSGCYQELLVQTEEQQQLDLLPNMHPENIFNSESSMFVYSSIPQRNSVHSLISVNTSSLAPDSAEGGGLSFVDTIGPSFDHSVETSLSYGMNSTDSTLPSSARSSVGLDLLPTVAAPASSVSSLETVINSKRQKLQFRQHNRIEELNSPHGIKAQAPVLLNYSSLPLVRGLSSGGNNSRPPKEPLIPGAQYITALLRLENSSCEDLCLPEWNEDELKDCRRIIRIERKRHSNEIVASFSIVGSATQNPQIKPASDSDVQVLEVSCLQCLVNHNETDEGKQGSSPNEIQHSSAEPPKSNLNRTLSSSSHRAAGSSVGYQYYITSVEVIKIIELLVGSHLIEDSQQKRKERGRVRSNLVPFWSKHPVSSSKKTLRPRSGPACNDDYLAELAHRIMTYEIRRPRIFDKNVRILEWAKLAPALQRALQSYYVAVPLDESENKLV

SEQ ID NO: 102 - protein, 450 aa, Komagataella pastoris
MLTAGSVQRLQISMFQTQEQEQVDLSSSIHTENIFNSESSGPAYPNIPQISSVHSFIGAKSSLLADAAPNQCLSFVDSAKVTSFDQSFHGSPSYKLNSAKLAVSSSEYLSVESSTEVDSTISLEAQVDRKRQKIQPERHSKTDNLNLFSDFDLLAPAFLTYYLPLFRGCSSGGNNKPPKKRSITGTQYVAASLRLESSTIEDICLPKWNENELKDNRRIIRIERRQQGNEIAASFSIVGSALENPHTKPTLDPDVEVLEVSCLRCLVNYDEIDDKQTVHGSLLHEVQSEFTEIRKQESTGKRSSYACTSNGTSIECQYYVTSVEVIKIIELLVGSHLIQDRKQRRKERGCIRSNLMPFWSKHLISSTTNTMKRRSVTASNENYIAELAHRIKTYEVRRPRKVNKDLRILEWSKLGPALRRALHSYYVAVPIDKFKNIAKPTNSELMFNIQ

SEQ ID NO: 103 - protein, 414 aa, Komagataella pastoris
MFQPLEQEQQLDLLFAMPPENIFNSDSSALTFSNTFQTSSVHSVTGVDSPSLVNINIPKSESFFMDSMTGASFGQTFQDLFPAFPLLKLPTMVAPPTSLKMVMDRKRRQSQITQQNTTENLNSFHGMNMPVPAFLEYSAFPLVRGFCSGGKIKPPKNQLVPGTEYVNVCLELLNVNYEGICLPKWNEKELEESRRIVRVEKRRQYNKIEAAFSIVDSVQENPQTNIAVDPNVDVLEFSCLLCPIKEHNSEVAQTVPTSSSSEEHKYSELAGHDFEENLSCGSDKEIETSVRFEYYITSVEVIRIIEFLTENHLIQDIQAKRRERSCLRSNLVRFWSKDIVPSSTSTMSKGSKPSRENDCIEELAQRIRAYKVRKPLIVDKSLRILEWSKLGPALERALQSYYVALPLDKIKTKL

SEQ ID NO: 104 - protein, 531 aa, Komagataella pastoris
MRLYLATVGTPFIGAPPVIASNEDHSTAHNNNSNDFNYECYNKEKLSGDLLEADLFDPPIDDTMHCSMFSSSTTADTEQLQKPKHESQHKNPFDCCQELKLKMADQQELHLLPEKITKNIFNSESPVFICTKIPQTSPVQSFVDVNASLLATNSALTTNSFSVDTTVDILFEQTVQDPYSYRLNSKETAPPSTASFFGTLDLGPTVEASTTLLEAAVNSKRRIVELRQQNRIEDLNFSTSMFLDHSRIPLVRGRSFGGNSSRPPKTPLIPGTRYVTARLRLENSTSEDICLPHWNKAELKDSRRIIRIERRYQSNEIVATFSIVGSAIEHPKTKPAFDPDVKVLEVSCLKCPTNENEPNEERFVSECTSDEARAQHALTEPLMSGFNREPNGRNPKTAGNSIGCRYYITSVEVIKIIELLIGSYSTSDPVQRRKERGRIRSNLVQFWSKHLVFSSKNSMKRRSVPTGNDAYLTELEHRINTYDVRKPRLFDKSVKILEWSKLGPALQRAIQSYYMVRLDNSSDITATTNSS

SEQ ID NO: 105 - protein, 513 aa, Komagataella pastoris
MQFYLATPTTPFVDNLTIPSGSGAYSKSNGNNSSSFGHSYNGKEELTDDLLTADFVDPPIDDAILYSMLSPFMNATTEQPQEFQNEQQHQNLSGCYQELLVQTEEQQQLDLLPNMHPENIFNSESSMFVYSSIPQRNSVHSLISVNTSSLAPDSAEGGGLSFVDTIGPSFDHSVETSLSYGMNSTDSTLPSSARSSVGLDLLPTVAAPASSVSSLETVINSKRQKLQFRQHNRIEELNSPHGIKAQAPVLLNYSSLPLVRGLSSGGNNSRPPKEPLIPGAQYITALLRLENSSCEDLCLPEWNEDELKDCRRIIRIERKRHSNEIVADSDVQVLEVSCLQCLVNHNETDEGKQGSSPNEIQHSSAEPPKSNLNRTLSSSSHRAAGSSVGYQYYITSVEVIKIIELLVGSHLIEDSQQKRKERGRVRSNLVPFWSKHPVSSSKKTLRPRSGPACNDDYLAELAHRIMTYEIRRPRIFDKNVRILEWAKLAPALQRALQSYYVAVPLDESENKLV

SEQ ID NO: 106 - protein, 521 aa, Komagataella pastoris
MLTAGSVQRLQISMFQTQEQEQVDLSSSIHTENIFNSESSGPAYPNIPQISSVHSFIGAKSSLLADAAPNQCLSFVDSAKVTSFDQSFHGSPSYKLNSAKLAVSSSEYLSVESSTEVDSTISLEAQVDRKRQKIQPERHSKTDNLNLFSDFDLLAPAFLTYYLPLFRGCSSGGNNKPPKKRSITGTQYVAASLRLESSTIEDICLPKWNENELKDNRRIIRIERRQQGNEIAASFSIVGSALENPHTKPTLDPDVEVLEVSCLRCLVNYDEIDDKQTVHGSLLHEVQSEFTEIRKQESTGKRSSYACTSNGTSIECQYYVTSVEVIKIIELLVGSHLIQDRKQRRKERGCIRSNLMPFWSKHLISSTTNTMKRRSVTASNENYIAELAHRIKTYEVRRPRKVNKDLRILEWSKLGPALRRALHSYYVAVPIDKFKNIAKPTNTSLGTIKMAIAGRSEVLETVFIARQSPVEPDACSSIANVRRELCDACTGTQANIVTEKILLTTIAADAGSTIVVPVCVP

SEQ ID NO: 107 - protein, 524 aa, Komagataella pastoris
MRLSLATRETPSTGTSPVFAGDEDHSSANNSSSNTFNYDYYIKEELSGNLLETAFFESPINDTMLCSMLSYPKIAGTEKIQEPDHELQQHRNPFGCCQELKAELAEQQELYLLPNKLTKNIFNSGSPVFIYTKIPQTSSVQSFVDVSNSLFATDSALPTSSFRVDSTVDISFEHSIQDSYCYRSNSAEIAPPSTALSSGTHDLVSTVEASTTLLEAAVNSKRRMLELRQQNRIEDLSFSTSIFLDHSRLPLVRGRSFGGNSSRPPKTPLIPGTRYVTARLRLENSTSEDMCLPQWNKAELKDSRRIIRIERRYQSDEIVASFSIVGSAIEYPKTKPAFDPDVKVLEVSCLKCPTNENEPNEERFVSECTSDEARTQHALTEPLTSGFNREPNGHNPKTAGNSIGCRYYITSVEVIKIIELLIGSYSTSDPVQRRKERGRIRSNLVQFWSKHLVFSSKNSMKRRSVPTGNDAYLTELEHRINTYDVRKPRLFDKSVKILEWSKLGPALRRAIQSYYMVRLDESSN

SEQ ID NO: 108 - protein, 368 aa, Artificial
  Synthetic
MLAEIGDYFRTNSSNGFSSSCYNKEKLSGDLLESDFVNSSFDDTTLHSMLSSSINADPEQSQKPQQRHKNQSSCHQELKIETEDQQEPYLLPDMLTKNIFNSQSSMFVYSSIPQISSVQSFIDTNTFSAVDGGAAPEGNSLSVDSTLGLSSDQSFEKLYSYDQNFEKEPQSPACPLVKCDPVSIMKTSTNSLETAVNTKRRMFQLRNQNEIESLNVSSDIVVATSKFLNHSWIPLIRGRNFGGSNCKPPKKPLFPGARYVTARLRLEYSSCKDMCLPEWNEYELEDSRRIIRIERLFDSNEIVASFSIVGSAVENPETRPVFNPNVKVLEVSCLRCLTNNNESDEENCVNKDLDGRNRDMVKDSFGCK

SEQ ID NO: 109 - protein, 400 aa, Artificial
  Synthetic
MNSSSTRGLQAISLTPTRVLFTLSTLFADTSKMSAENGDYFRTNSSNEFSNNCFNKEKLSEDLLEASFVDSPIEDTIPHFMLSSSINADSEQPQEPQHRHKNQSSCHRELKIETNDQQEPYLLPDMLTKNIFNSQSSMFVYSSIPQTSSVQSFIDTNIFSAVDGGAAPEENSLSVDSTLGPSSDQSFEKLYSYDQNFEKEPQSPACPLVKCDLVGIMKASTNSLEVAVNTKRQMFQLRHQNEIESLYVSSDIVIATSNFLDHSWIPLIRGRNFGGSNCKPPKKPLFPGARYVTARLRLEYSSCEDMCLPEWNEYELKDSRRIIRLERRFDSNEIVASFSIVGSAVENPETRPAFNPNVKVLEVSCLRCLTNNNESDEENCVNKDLDGRVHNMVKDSIGCK

SEQ ID NO: 110 - protein, 405 aa, Artificial
  Synthetic
MRLYLATVGTPFIGAPPVIASNEDHSTAHNNNSNDFNYECYNKEKLSGDLLEADLFDPPIDDTMHCSMFSSSTTADTEQLQKPKHESQHKNPFDCCQELKLKMADQQELHLLPEKITKNIFNSESPVFICTKIPQTSPVQSFVDVNASLLATNSALTTNSFSVDTTVDILFEQTVQDPYSYRLNSKETAPPSTASFFGTLDLGPTVEASTTLLEAAVNSKRRIVELRQQNRIEDLNFSTSMFLDHSRIPLVRGRSFGGNSSRPPKTPLIPGTRYVTARLRLENSTSEDICLPHWNKAELKDSRRIIRIERRYQSNEIVATFSIVGSAIEHPKTKPAFDPDVKVLEVSCLKCPTNENEPNEERFVSECTSDEARAQHALTEPLMSGFNREPNGRNPKTAGNSIGCR

SEQ ID NO: 111 - protein, 406 aa, Artificial
  Synthetic
MRLSLATRETPSTGTSPVFAGDEDHSSANNSSSNTFNYDYYIKEELSGNLLETAFFESPINDTMLCSMLSYPKIAGTEKIQEPDHELQQHRNPFGCCQELKAELAEQQELYLLPNKLTKNIFNSGSPVFIYTKIPQTSSVQSFVDVSNSLFATDSALPTSSFRVDSTVDISFEHSIQDSYCYRSNSAEIAPPSTALSSGTHDLVSTVEASTTLLEAAVNSKRRMLELRQQNRIEDLSFSTSIFLDHSRLPLVRGRSFGGNSSRPPKTPLIPGTRYVTARLRLENSTSEDMCLPQWNKAELKDSRRIIRIERRYQSDEIVASFSIVGSAIEYPKTKPAFDPDVKVLEVSCLKCPTNENEPNEERFVSECTSDEARTQHALTEPLTSGFNREPNGHNPKTAGNSIGCR

SEQ ID NO: 112 - protein, 389 aa, Artificial
  Synthetic
MQLLSASSSTPFVAMSTVNAGNECYLRTNNSNSNGISDNRFRKKKLSEDLLKADFVEDTAGNPMIYPMVLSAPTYIKTEHLQELEHKQQGSNLLSEMFTKNIFNSHSAMFVYSEIPQISSVQSFIGSDTSVLVADASSEKNSLFVYSNTGISFDPSDEDPCPYEANFDPGQLFSAVSAVEDGSMATLTAPTMSLEGVVNSKKGICQLRQHNRIEGFDSSHEFKVLGSMFLNHSYVPLVRGRSFGGNNCRPPKEPLIPGTRYVAAHLRLENNTCEDMCLPEWNKHELEDSRRIIRIERRYQSNEIVASFSIVGSAKENPQTKPSSDPDVKVLEVSCLKCFINGNESDEQFVTECQSDEAHYGFTELPKQNFSRKSKGCSHKMANNTIACQ

SEQ ID NO: 113 - protein, 411 aa, Artificial
  Synthetic
MQLFLATLGIPFTGTLPAIAGNEDHSSANNNNGNGFSYDWHGNDTLFAELLETDFFDHPIDDAMLCSLLSPPRAAETEQQQDTEWEQQKHTNLFGCCQKHKVEMAKQQELYLLPDKLTKNIFNSESPVFICTKIPQTSSVQSFVDVDASLLATDSSLSKDSFSADSTVDVLFEQSVQEPYFYRSNSAETAPPSTACSSATLDLVSTLEATTTLFEEAANSKKQMIGLKQQSKIEDLSFSCKLNFSASMFLDHSCIPLVRGRSFGGNSRPPKTPLIPGTRYVTARLRLENSTSEDMCFPHWSERELSDSRRIIRIERSYQSNEIVASFFIVGSAIDHPETKPSSELDVKVLEVSCLRCFINDNESEEERFVSEYTSDEARAQHALTEPLTSGLNKEPNGHNPKTAGNSIGCR

SEQ ID NO: 114 - protein, 409 aa, Artificial
  Synthetic
MQFYLATPTTPFVDNLTIPSGSGAYSKSNGNNSSSFGHSYNGKEELTDDLLTADFVDPPIDDAILYSMLSPFMNATTEQPQEFQNEQQHQNLSGCYQELLVQTEEQQQLDLLPNMHPENIFNSESSMFVYSSIPQRNSVHSLISVNTSSLAPDSAEGGGLSFVDTIGPSFDHSVETSLSYGMNSTDSTLPSSARSSVGLDLLPTVAAPASSVSSLETVINSKRQKLQFRQHNRIEELNSPHGIKAQAPVLLNYSSLPLVRGLSSGGNNSRPPKEPLIPGAQYITALLRLENSSCEDLCLPEWNEDELKDCRRIIRIERKRHSNEIVASFSIVGSATQNPQIKPASDSDVQVLEVSCLQCLVNHNETDEGKQGSSPNEIQHSSAEPPKSNLNRTLSSSSHRAAGSSVGYQ

SEQ ID NO: 115 - protein, 317 aa, Artificial
  Synthetic
MLTAGSVQRLQISMFQTQEQEQVDLSSSIHTENIFNSESSGPAYPNIPQISSVHSFIGAKSSLLADAAPNQCLSFVDSAKVTSFDQSFHGSPSYKLNSAKLAVSSSEYLSVESSTEVDSTISLEAQVDRKRQKIQPERHSKTDNLNLFSDFDLLAPAFLTYYLPLFRGCSSGGNNKPPKKRSITGTQYVAASLRLESSTIEDICLPKWNENELKDNRRIIRIERRQQGNEIAASFSIVGSALENPHTKPTLDPDVEVLEVSCLRCLVNYDEIDDKQTVHGSLLHEVQSEFTEIRKQESTGKRSSYACTSNGTSIECQ

SEQ ID NO: 116 - protein, 293 aa, Artificial
  Synthetic
MFQPLEQEQQLDLLFAMPPENIFNSDSSALTFSNTFQTSSVHSVTGVDSPSLVNINIPKSESFFMDSMTGASFGQTFQDLFPAFPLLKLPTMVAPPTSLKMVMDRKRRQSQITQQNTTENLNSFHGMNMPVPAFLEYSAFPLVRGFCSGGKIKPPKNQLVPGTEYVNVCLELLNVNYEGICLPKWNEKELEESRRIVRVEKRRQYNKIEAAFSIVDSVQENPQTNIAVDPNVDVLEFSCLLCPIKEHNSEVAQTVPTSSSSEEHKYSELAGHDFEENLSCGSDKEIETSVRFE

SEQ ID NO: 117 - protein, 405 aa, Artificial; identical to SEQ ID NO: 110
  Synthetic

SEQ ID NO: 118 - protein, 391 aa, Artificial
  Synthetic
MQFYLATPTTPFVDNLTIPSGSGAYSKSNGNNSSSFGHSYNGKEELTDDLLTADFVDPPIDDAILYSMLSPFMNATTEQPQEFQNEQQHQNLSGCYQELLVQTEEQQQLDLLPNMHPENIFNSESSMFVYSSIPQRNSVHSLISVNTSSLAPDSAEGGGLSFVDTIGPSFDHSVETSLSYGMNSTDSTLPSSARSSVGLDLLPTVAAPASSVSSLETVINSKRQKLQFRQHNRIEELNSPHGIKAQAPVLLNYSSLPLVRGLSSGGNNSRPPKEPLIPGAQYITALLRLENSSCEDLCLPEWNEDELKDCRRIIRIERKRHSNEIVADSDVQVLEVSCLQCLVNHNETDEGKQGSSPNEIQHSSAEPPKSNLNRTLSSSSHRAAGSSVGYQ

SEQ ID NO: 119 - protein, 317 aa, Artificial; identical to SEQ ID NO: 115
  Synthetic

SEQ ID NO: 120 - protein, 406 aa, Artificial; identical to SEQ ID NO: 111
  Synthetic

(19) United States
(12) Patent Application Publication
(10) Pub. No.: US 2019/0010194 A1
(43) Pub. Date: Jan. 10, 2019

(54) METHANOL-UTILIZING YEAST-DERIVED NOVEL PROTEIN AND METHOD FOR PRODUCING PROTEIN OF INTEREST USING SAME

(71) Applicant: KANEKA CORPORATION, Osaka (JP)

(72) Inventor: Teruyuki Nishi, Hyogo (JP)

(73) Assignee: KANEKA CORPORATION, Osaka (JP)

(21) Appl. No.: 16/142,501

(22) Filed: Sep. 26, 2018

Related U.S. Application Data
(63) Continuation of application No. PCT/JP2017/012510, filed on Mar. 28, 2017.

(30) Foreign Application Priority Data
Mar. 28, 2016 (JP) 2016-064364

(51) Int. Cl. C07K 14/39; C12N 15/62; C12N 1/16; C12P 21/02

(52) U.S. Cl. CPC C07K 14/39; C12N 15/62; C07K 2317/24; C12P 21/02; C12N 1/16

(57) ABSTRACT

A vector includes a nucleotide sequence encoding an amino acid sequence selected from the group consisting of SEQ ID NOs: 113, 108 to 112, and 114 to 120, or a variant thereof. A vector includes a nucleotide sequence encoding an amino acid sequence selected from the group consisting of SEQ ID NOs: 100, 95 to 99, and 101 to 107, or a variant thereof. A method for preparing a mutant strain includes introducing such a vector into a host cell, and obtaining a mutant strain including the vector. The mutant strain has an increased secretion amount of a protein compared to a secretion amount of the host cell before the introduction.

### TECHNICAL FIELD

One or more embodiments of the present invention relate to a vector comprising a nucleotide sequence encoding a novel protein derived from a methanol-utilizing yeast, a mutant cell expressing a gene encoding the protein at a high level, and a method for producing a protein of interest using the mutant cell as a host.

### BACKGROUND

Gene recombination methods are widely used for the production of industrially useful biomaterials such as antibodies, enzymes, and cytokines to be utilized in the medical treatment and diagnosis uses. Hosts used for producing a protein of interest by the gene recombination method are animals such as chicken and cow, animal cells such as CHO, insects such as silkworm, insect cells such as sf9, as well as microorganisms such as yeasts, E. coli, and actinomycetes. Yeasts, among the host organisms, are extremely beneficial and various studies have been conducted, since: large scale culture is possible in a low-cost medium at a high density, and thus the protein of interest may be produced at a low cost; a secretory expression into a culture medium is feasible with the use of a signal peptide, and thus the purification process of a protein of interest may be easy; and post-translational modifications such as glycosylation can be made due to being an eukaryote. If an innovative production technology which can be applied to various proteins of interest in yeasts is developed, diverse industrial expansions in addition to the improvement of cost competitiveness by the significantly improved productivity can be hopefully expected.

Komagataella pastoris, a yeast species, is a methanol-utilizing yeast having a excellent protein expression ability and capable of utilizing a low-cost carbon source, which is advantageous in the industrial production. For example, Non Patent Literature 1 reports a method for producing exogenous proteins using Komagataella pastoris such as green fluorescent protein, human serum albumin, hepatitis B virus surface antigen, human insulin, and single-chain antibody. For the production of an exogenous protein in a yeast, various attempts have been made to improve the productivity thereof such as addition of a signal sequence, use of a strong promoter, codon modification, co-expression of chaperone genes, co-expression of transcription factor genes, inactivation of a protease gene derived from a host yeast, and studies on culture conditions. For example, Patent Literature 1 reports the productivity improvement by the co-expression of transcription factors that activate the AOX promoter. Additionally, Non Patent Literatures 2 to 4 report the productivity improvement by the codon modification in consideration of the codon usage frequency of Komagataella pastoris, co-expression of chaperone genes, and inactivation of a protease gene, respectively.

However, all Literatures aim at improving the production of exogenous proteins but do not aim at searching factors for improving the secretion of both an endogenous protein and an exogenous protein.

### CITATION LIST

### Patent Literature

Patent Literature 1: International Publication No. WO2012/102171

### Non Patent Literature

Non Patent Literature 1: FEMS Microbiology Reviews 24 (2000) 45-66
Non Patent Literature 2: PLoS One. 2011; 6(8): e22577
Non Patent Literature 3: Biotechnol. Bioeng. 2006 Mar. 5; 93(4):771-8
Non Patent Literature 4: Methods Mol. Biol. 103, 81-94

### SUMMARY

One or more embodiments of the present invention provide a new means for improving secretion amounts of an endogenous protein and an exogenous protein.

The present inventors identified a novel protein family by a comprehensive analysis on the nucleotide sequence of chromosomal DNA of yeasts belonging to the genus Komagataella. The inventors further found that secretion amounts of endogenous proteins and an exogenous protein in yeasts belonging to the genus Komagataella are improved by expressing the gene encoding the novel protein at a high level.

More specifically, one or more embodiments of the present invention encompass the following aspects.

(1) A vector comprising:

(a) a nucleotide sequence encoding an amino acid sequence as set forth in any of SEQ ID NOs: 113, 108 to 112, and 114 to 120,

(b) a nucleotide sequence encoding an amino acid sequence in which one or more amino acids are substituted, deleted, and/or added in the amino acid sequence as set forth in the (a),

(c) a nucleotide sequence encoding an amino acid sequence having a sequence identity of 85% or more to the amino acid sequence as set forth in the (a), or

(d) a nucleotide sequence of a nucleic acid that hybridizes under stringent conditions to a nucleic acid consisting of a complementary sequence to a nucleotide sequence encoding the amino acid sequence as set forth in the (a).

(2) The vector according to (1), wherein the nucleotide sequences according to the (a) to (d) are respectively:

(a′) a nucleotide sequence as set forth in any of SEQ ID NOs: 75, 65, 67, 69, 71, 73, 77, 79, 81, 83, 85, 87, and 89,

(b′) a nucleotide sequence in which one or more nucleotides are substituted, deleted, and/or added in the nucleotide sequence as set forth in the (a′),

(c′) a nucleotide sequence having a sequence identity of 85% or more to the nucleotide sequence as set forth in the (a′), and

(d′) a nucleotide sequence of a nucleic acid that hybridizes under stringent conditions to a nucleic acid consisting of a complementary sequence to the nucleotide sequence as set forth in the (a′).

(3) A vector comprising:

(e) a nucleotide sequence encoding an amino acid sequence as set forth in any of SEQ ID NOs: 100, 95 to 99, and 101 to 107,

(f) a nucleotide sequence encoding an amino acid sequence in which one or more amino acids are substituted, deleted, and/or added in the amino acid sequence as set forth in the (e),

(g) a nucleotide sequence encoding an amino acid sequence having a sequence identity of 85% or more to the amino acid sequence as set forth in the (e), or

(h) a nucleotide sequence of a nucleic acid that hybridizes under stringent conditions to a nucleic acid consisting of a complementary sequence to a nucleotide sequence encoding the amino acid sequence as set forth in the (e).

(4) The vector according to (3), wherein the nucleotide sequences according to the (e) to (h) are respectively:

(e′) a nucleotide sequence as set forth in any of SEQ ID NOs: 39, 24, 27, 30, 33, 36, 42, 45, 48, 51, 54, 57, and 60,

(f′) a nucleotide sequence in which one or more nucleotides are substituted, deleted, and/or added in the nucleotide sequence as set forth in the (e′),

(g′) a nucleotide sequence having a sequence identity of 85% or more to the nucleotide sequence as set forth in the (e′), and

(h′) a nucleotide sequence of a nucleic acid that hybridizes under stringent conditions to a nucleic acid consisting of a complementary sequence to the nucleotide sequence as set forth in the (e′).

(5) The vector according to any of (1) to (4), wherein the vector increases a secretion amount of a protein in a host cell.
(6) A protein secretion enhancer consisting of the vector according to any of (1) to (5).
(7) A mutant cell having an increased expression of a gene comprising the nucleotide sequence defined in any of (1) to (4) compared to that of a wild-type, and an increased secretion amount of a protein compared to that of the wild-type gene.
(8) A cell comprising the vector according to any of (1) to (5).
(9) The cell according to (7) or (8), wherein the cell is a yeast, a bacterium, a fungus, an insect cell, an animal cell, or a plant cell.
(10) The cell according to (9), wherein the yeast is a methanol-utilizing yeast, a fission yeast, or a budding yeast.
(11) The cell according to (10), wherein the methanol-utilizing yeast is a yeast belonging to the genus Komagataella or a yeast belonging to the genus Ogataea.
(12) A method for preparing a mutant strain, comprising a step of introducing the vector according to any of (1) to (5) or the protein secretion enhancer according to (6) into a host cell, wherein the mutant strain has an increased secretion amount of a protein compared to the that of host cell before the introduction.
(13) A method for producing a protein of interest, comprising:

a step of culturing the cell according to any of (7) to (11), and

a step of recovering the protein of interest from a culture medium.

(14) The method according to (13), wherein the protein of interest is a heterologous protein.
(15) The method according to (13) or (14), wherein a culture medium comprising one or more carbon sources selected from the group consisting of glucose, glycerol and methanol is used in the step of culturing.

The present specification encompasses the content disclosed in JP Patent Application No. 2016-064364, to which present application claims priority.

Secretion amounts of an endogenous protein and an exogenous protein in a host cell are improved by expressing the polypeptide according to one or more embodiments of the present invention at a high level. Further, one or more embodiments of the present invention provide a method for effectively producing a protein of interest using a yeast belonging to the genus Komagataella as a host.

### BRIEF DESCRIPTION OF DRAWINGS

FIGS. 1A to 1E show a diagram showing the alignment result of the amino acid sequences of novel polypeptides discovered by the present inventors. FIG. 1B is the continuation of FIG. 1A. FIG. 1C is the continuation of FIG. 1B. The arrow indicates the start site of C-terminal regions found to have relatively high homology to one another that were deleted in the Examples. FIG. 1D is the continuation of FIG. 1C. FIG. 1E is the continuation of FIG. 1D.

### DETAILED DESCRIPTION OF EMBODIMENT

Hereinafter, one or more embodiments of the present invention are described in detail.

### 1. Definition of Terms

In one or more embodiments of the present invention, two nucleic acids hybridizing under stringent conditions means, for example, as follows. For example, nucleic acid Y is considered as “the nucleic acid that hybridizes to the nucleic acid X under the stringent conditions”, when the nucleic acid Y can be obtained as the nucleic acid bound on a filter by using the filter with an immobilized nucleic acid X, hybridizing it to a nucleic acid Y at 65° C. in the presence of 0.7 to 1.0 M NaCl, and then washing the filter under the condition of 65° C. using 2-fold concentration of an SSC solution (the composition of 1-fold concentration of the SSC solution consists of 150 mM sodium chloride and 15 mM sodium citrate) Alternatively, the nucleic acid X and the nucleic acid Y can be said to “hybridize to each other under stringent conditions.” The nucleic acid Y may be a nucleic acid obtained as the nucleic acid bound on a filter by washing the filter at 65° C. using 0.5-fold concentration of an SSC solution, washing at 65° C. using 0.2-fold concentration of an SSC solution, or washing at 65° C. using 0.1-fold concentration of an SSC solution. The standard nucleic acid X may be a colony- or plaque-derived nucleic acid X.

The sequence identity of a nucleotide sequence and an amino acid sequence in one or more embodiments of the present invention can be determined by a method or a sequence analysis software known by a person skilled in the art. Examples include the blastn program and blastp program of BLAST algorithm, and fasta program of FASTA algorithm. In one or more embodiments of the present invention, the “sequence identity” of a certain nucleotide sequence to be evaluated to a nucleotide sequence X is a value shown in % of the frequency with which same nucleotides appear in same sites of the nucleotide sequences including the gap parts, when the nucleotide sequence X and the nucleotide sequence to be evaluated are aligned and gaps are introduced as needed to achieve the highest nucleotide alignment between both sequences. When comparing a nucleotide sequence of DNA with a nucleotide sequence of RNA, T and U are considered as the identical nucleotide. In one or more embodiments of the present invention, the “sequence identity” of a certain amino acid sequence to be evaluated to an amino acid sequence X is a value shown in % of the frequency with which the same amino acid appears in the same site of the amino acid sequences including the gap parts, when the amino acid sequence X and the amino acid sequence to be evaluated are aligned and gaps are introduced as needed to achieve the highest amino acid alignment between both sequences.

The “nucleic acid” in one or more embodiments of the present invention may also be called as “polynucleotide” referring to DNA or RNA, but typically referring to DNA. The “polynucleotide” in one or more embodiments of the present invention may be present in the double-stranded form with a complementary strand thereto. In particular, when the “polynucleotide” is DNA, it may be preferable that the DNA comprising a certain nucleotide sequence be present in the double-stranded form with DNA comprising a complementary nucleotide sequence thereto.

In one or more embodiments of the present invention, the “polypeptide” refers to those in which 2 or more amino acids are peptide bonded, and includes those having a short chain length called as peptides and oligopeptides in addition to proteins.

The “nucleotide sequence encoding a polypeptide” in one or more embodiments of the present invention refers to a nucleotide sequence of a polynucleotide that produces a polypeptide by transcription and translation, and refers to, for example, a nucleotide sequence designed based on a codon table to a polypeptide consisting of an amino acid sequence.

The “host cell” in one or more embodiments of the present invention refers to a cell to be transformed by introducing a vector thereinto, and called as a “host” or a “transformant”. Herein, a host cell before and after transformation is sometimes simply called as the “cell.” The cell used as the host is not particularly limited as long as a vector can be introduced to the cell.

The species of the host cell is not particularly limited, and examples include a yeast, a bacterium, a fungus, an insect cell, an animal cell, and a plant cell, including yeast such as a methanol-utilizing yeast. The methanol-utilizing yeast is generally defined as a yeast which can be cultured by utilizing methanol as an only carbon source, but yeast which originally was a methanol-utilizing yeast but lost the methanol-utilizing ability due to an artificial modification or mutation is also encompassed by the methanol-utilizing yeast of one or more embodiments of the present invention.

Examples of the methanol-utilizing yeast include yeasts belonging to the genus Pichia, the genus Ogataea, the genus Candida, the genus Torulopsis, and the genus Komagataella. Examples include Pichia methanolica in the genus Pichia, Ogataea angusta, Ogataea polymorpha, Ogataea parapolymorpha, and Ogataea minuta in the genus Ogataea, Candida boidinii in the genus Candida, Komagataella pastoris and Komagataella phaffii in the genus Komagataella.

Among the methanol-utilizing yeasts described above, yeasts belonging to the genus Komagataella or yeasts belonging to the genus Ogataea may be used.

For the yeast belonging to the genus Komagataella, Komagataella pastoris and Komagataella phaffii may be used. Komagataella pastoris and Komagataella phaffii both have another name as Pichia pastoris.

Specific examples of the strain to be used as the host include strains of Komagataella pastoris ATCC76273 (Y-11430, CBS7435) and Komagataella pastoris X-33. These strains are available from American Type Culture Collection and Thermo Fisher Scientific, Inc.

For the yeast belonging to the genus Ogataea, Ogataea angusta, Ogataea polymorpha, and Ogataea parapolymorpha may be used. These 3 are closely related to each other, and all are also known as Hansenula polymorpha or Pichia angusta.

Specific examples of the strain to be used include Ogataea angusta NCYC495 (ATCC14754), Ogataea polymorpha 8V (ATCC34438), and Ogataea parapolymorpha DL-1 (ATCC26012). These strains are available from American Type Culture Collection.

Further, in one or more embodiments of the present invention, derivative strains from these strains of yeasts belonging to the genus Komagataella or yeasts belonging to the genus Ogataea can also be used, and examples of histidine-dependent yeasts include Komagataella pastoris GS115 strain (available from Thermo Fisher Scientific, Inc.), and examples of leucine-dependent yeasts include NCYC495-derived BY4329, 8V-derived BY5242, and DL-1-derived BY5243 (these can be distributed from National BioResource Project). In one or more embodiments of the present invention, derivative strains from these strains can also be used.

The “expression” in one or more embodiments of the present invention refers to the transcription and translation of the nucleotide sequence that produces a polypeptide. Further, the expression may be in a substantially constant state depending or without depending on external stimulation or growth conditions. The promoter for driving the expression is not particularly limited as long as the promoter drives the expression of a nucleotide sequence encoding a polypeptide.

The “expression at a high level” in one or more embodiments of the present invention means an increased amount of a polypeptide in a host cell or an increased amount of mRNA in a host cell compared to a normal amount, and, for example, such a level can be confirmed by measuring an amount by utilizing an antibody that recognizes the polypeptide or measuring amounts by the RT-PCR method, northern hybridization, or the hybridization using DNA array, and comparing the amount to that of non-modified strains such as a parent cell or a wild-type strain.

### 2. Vector Comprising a Nucleotide Sequence Encoding a Novel Polypeptide

One or more embodiments of the present invention relate to a vector comprising (a) a nucleotide sequence encoding an amino acid sequence as set forth in any of SEQ ID NOs: 113, 108 to 112, and 114 to 120, for example SEQ ID NOs: 113, 108 to 112, 117, and 120, (b) a nucleotide sequence encoding an amino acid sequence in which one or more amino acids are substituted, deleted, and/or added in the amino acid sequence as set forth in the (a), (c) a nucleotide sequence encoding an amino acid sequence having a sequence identity of 85% or more, for example, 90% or more, 95% or more, 96% or more, 97% or more, 98% or more, or 99% or more, to the amino acid sequence as set forth in the (a), or (d) a nucleotide sequence of a nucleic acid that hybridizes under stringent conditions to a nucleic acid consisting of a complementary sequence to a nucleotide sequence encoding the amino acid sequence as set forth in the (a).

The “one or more” regarding the substitution, deletion, insertion and/or addition of amino acids in one or more embodiments of the present invention means, for example, in the amino acid sequence according to (b), 1 to 280 amino acids, 1 to 250 amino acids, 1 to 200 amino acids, 1 to 190 amino acids, 1 to 160 amino acids, 1 to 130 amino acids, 1 to 100 amino acids, 1 to 75 amino acids, 1 to 50 amino acids, 1 to 25 amino acids, 1 to 20 amino acids, 1 to 15 amino acids, 1 to 10 amino acids, 1 to 7 amino acids, 1 to 5 amino acids, 1 to 4 amino acids, 1 to 3 amino acids, or 1 or 2 amino acids, in the amino acid sequence as set forth in any of SEQ ID NOs: 113, 108 to 112, and 114 to 120. Examples of the amino acid sequence according to (b) include partial amino acid sequences consisting of 1 to 5, 1 to 10, 1 to 25, 1 to 50, 1 to 75, 1 to 100, 1 to 125, 1 to 150, 1 to 175, 1 to 200, 1 to 225, 1 to 250, 1 to 275, or 1 to 300 consecutive amino acids in the amino acid sequence as set forth in any of SEQ ID NOs: 113, 108 to 112, and 114 to 120.

In one embodiment, the nucleotide sequences according to (a) to (d) may be (a′) a nucleotide sequence as set forth in any of SEQ ID NOs:75, 65, 67, 69, 71, 73, 77, 79, 81, 83, 85, 87, and 89, for example SEQ ID NOs: 75, 65, 67, 69, 71, 73, 83, and 89, (b′) a nucleotide sequence in which one or more amino acids are substituted, deleted, and/or added in the amino acid sequence as set forth in the (a′), (c′) a nucleotide sequence having a sequence identity of 85% or more, for example, 90% or more, 95% or more, 96% or more, 97% or more, 98% or more, or 99% or more, to the nucleotide sequence as set forth in the (a′), or (d′) a nucleotide sequence of a nucleic acid that hybridizes under stringent conditions to a nucleic acid consisting of a complementary sequence to the nucleotide sequence as set forth in the (a′), respectively.

The “one or more” regarding the substitution, deletion, insertion and/or addition of nucleotides in one or more embodiments of the present invention means, for example, in the nucleotide sequence according to (b′), 1 to 800 nucleotides, 1 to 700 nucleotides, 1 to 600 nucleotides, 1 to 500 nucleotides, 1 to 400 nucleotides, 1 to 300 nucleotides, 1 to 200 nucleotides, 1 to 190 nucleotides, 1 to 160 nucleotides, 1 to 130 nucleotides, 1 to 100 nucleotides, 1 to 75 nucleotides, 1 to 50 nucleotides, 1 to 25 nucleotides, 1 to 20 nucleotides, 1 to 15 nucleotides, 1 to 10 nucleotides, 1 to 7 nucleotides, 1 to 5 nucleotides, 1 to 4 nucleotides, 1 to 3 nucleotides, or 1 or 2 nucleotides in the sequence as set forth in any of SEQ ID NOs: 75, 65, 67, 69, 71, 73, 77, 79, 81, 83, 85, 87, and 89. Examples of the nucleotide sequence of (b′) include partial nucleotide sequences consisting of 1 to 5, 1 to 10, 1 to 25, 1 to 50, 1 to 75, 1 to 100, 1 to 125, 1 to 150, 1 to 175, 1 to 200, 1 to 225, 1 to 250, 1 to 275, 1 to 300, 1 to 350, 1 to 400, 1 to 450, 1 to 500, 1 to 600, 1 to 700, or 1 to 800 consecutive nucleotides in the nucleotide sequence as set forth in any of SEQ ID NOs: 75, 65, 67, 69, 71, 73, 77, 79, 81, 83, 85, 87, and 89.

One or more embodiments of the present invention relate to a vector comprising (e) a nucleotide sequence encoding an amino acid sequence as set forth in any of SEQ ID NOs: 100, 95 to 99, and 101 to 107, (f) a nucleotide sequence encoding an amino acid sequence in which one or more amino acids are substituted, deleted, and/or added in the amino acid sequence as set forth in the (e), (g) a nucleotide sequence encoding an amino acid sequence having a sequence identity of 85% or more, for example, 90% or more, 95% or more, 96% or more, 97% or more, 98% or more, or 99% or more, to the amino acid sequence as set forth in the (e), or (h) a nucleotide sequence of a nucleic acid that hybridizes under stringent conditions to a nucleic acid consisting of a complementary sequence to a nucleotide sequence encoding the amino acid sequence as set forth in the (e).

The “one or more” regarding the substitution, deletion, insertion and/or addition of amino acids in one or more embodiments of the present invention means, for example, in the amino acid sequence according to (f), 1 to 400 amino acids, 1 to 300 amino acids, 1 to 200 amino acids, 1 to 190 amino acids, 1 to 160 amino acids, 1 to 130 amino acids, 1 to 100 amino acids, 1 to 75 amino acids, 1 to 50 amino acids, 1 to 25 amino acids, 1 to 20 amino acids, 1 to 15 amino acids, 1 to 10 amino acids, 1 to 7 amino acids, 1 to 5 amino acids, 1 to 4 amino acids, 1 to 3 amino acids, or 1 or 2 amino acids in the amino acid sequence as set forth in any of SEQ ID NOs: 100, 95 to 99, and 101 to 107. Examples of the amino acid sequence according to (f) include partial amino acid sequences consisting of 1 to 5, 1 to 10, 1 to 25, 1 to 50, 1 to 75, 1 to 100, 1 to 125, 1 to 150, 1 to 175, 1 to 200, 1 to 225, 1 to 250, 1 to 275, 1 to 300, 1 to 325, 1 to 350, 1 to 375, or 1 to 400 consecutive amino acids in the amino acid sequence as set forth in any of SEQ ID NOs: 100, 95 to 99, and 101 to 107.

In one embodiment, the nucleotide sequences according to (e) to (h) may be (e′) a nucleotide sequence as set forth in any of SEQ ID NOs: 39, 24, 27, 30, 33, 36, 42, 45, 48, 51, 54, 57, and 60, (f′) a nucleotide sequence in which one or more nucleotides are substituted, deleted, and/or added in the nucleotide sequence as set forth in the (e′), (g′) a nucleotide sequence having a sequence identity of 85% or more, for example, 90% or more, 95% or more, 96% or more, 97% or more, 98% or more, or 99% or more, to the nucleotide sequence as set forth in the (e′), or (h′) a nucleotide sequence of a nucleic acid that hybridizes under stringent conditions to a nucleic acid consisting of a complementary sequence to the nucleotide sequence as set forth in the (e′), respectively.

The “one or more” regarding the substitution, deletion, insertion and/or addition of nucleotides in one or more embodiments of the present invention means, for example, in the nucleotide sequence according to (f′), 1 to 1200 nucleotides, 1 to 1000 nucleotides, 1 to 500 nucleotides, 1 to 400 nucleotides, 1 to 300 nucleotides, 1 to 200 nucleotides, 1 to 190 nucleotides, 1 to 160 nucleotides, 1 to 130 nucleotides, 1 to 100 nucleotides, 1 to 75 nucleotides, 1 to 50 nucleotides, 1 to 25 nucleotides, 1 to 20 nucleotides, 1 to 15 nucleotides, 1 to 10 nucleotides, 1 to 7 nucleotides, 1 to 5 nucleotides, 1 to 4 nucleotides, 1 to 3 nucleotides, or 1 or 2 nucleotides in the nucleotide sequence as set forth in any of SEQ ID NOs: 39, 24, 27, 30, 33, 36, 42, 45, 48, 51, 54, 57, and 60. Examples of the nucleotide sequence of (f′) include partial nucleotide sequences consisting of 1 to 5, 1 to 10, 1 to 25, 1 to 50, 1 to 75, 1 to 100, 1 to 125, 1 to 150, 1 to 175, 1 to 200, 1 to 225, 1 to 250, 1 to 275, 1 to 300, 1 to 350, 1 to 400, 1 to 450, 1 to 500, 1 to 600, 1 to 700, 1 to 800, 1 to 900, 1 to 1000, 1 to 1100, or 1 to 1200 consecutive nucleotides in the nucleotide sequence as set forth in any of 39, 24, 27, 30, 33, 36, 42, 45, 48, 51, 54, 57, and 60.

The vector of one or more embodiments of the present invention may comprise a combination of 2 or more of the nucleotide sequences as set forth in the (a) to (h). Examples of the number of combination include 3 or more, 4 or more, 5 or more, 6 or more, 7 or more, 8 or more, 9 or more, or 10 or more. For example, the vector of one or more embodiments of the present invention may comprise a combination of 2 or more sequences of the (a), i.e., the nucleotide sequence encoding the amino acid sequence as set forth in any of SEQ ID NOs: 113, 108 to 112, and 114 to 120, or equivalent sequences to any of these sequences. Herein, the “equivalent sequence” to the sequences according to the (a) means the sequences according to the (b) to (d) to the respective sequences according to the (a). Thus, the vector of one or more embodiments of the present invention may comprise, for example, a combination of 2 or more sequences as set forth in the (a), a combination of 2 or more sequences as set forth in the (b), a combination of 2 or more sequences as set forth in the (c), or a combination of 2 or more sequences as set forth in the (d). Similarly, the vector of one or more embodiments of the present invention may comprise a combination of 2 or more sequences of the (e), i.e., a nucleotide sequence encoding the amino acid sequence as set forth in any of SEQ ID NOs: 100, 95 to 99, and 101 to 107, or equivalent sequences to any of these. Herein, the “equivalent sequence” to the sequences according to the (e) means the sequences according to the (f) to (h) to the respective sequences according to the (e). Thus, the vector of one or more embodiments of the present invention may comprise, for example, a combination of 2 or more sequences as set forth in the (e), a combination of 2 or more sequences as set forth in the (f), a combination of 2 or more sequences as set forth in the (g), or a combination of 2 or more sequences as set forth in the (h). The vector of one or more embodiments of the present invention may comprise, for example, a combination of 2 or more nucleotide sequences encoding the amino acid sequence as set forth in any of SEQ ID NOs: 113, 108 and 109, or equivalent sequences to any of these. The vector of one or more embodiments of the present invention may comprise a combination of 2 or more nucleotide sequences encoding the amino acid sequence as set forth in any of SEQ ID NOs: 100, 95, and 96, or equivalent sequences to any of these.

Further, the vector of one or more embodiments of the present invention may comprise a combination of 2 or more nucleotide sequences as set forth in the (a′) to (h′). Examples of the number of combination include 3 or more, 4 or more, 5 or more, 6 or more, 7 or more, 8 or more, 9 or more, or 10 or more. For example, the vector of one or more embodiments of the present invention may comprise a combination of 2 or more sequences of the (a′), i.e., the nucleotide sequence as set forth in any of SEQ ID NOs: 75, 65, 67, 69, 71, 73, 77, 79, 81, 83, 85, 87, and 89, or equivalent sequences to any of these sequences. Herein, the “equivalent sequence” to the sequences according to the (a′) means the sequences according to the (b′) to (d′) to the respective sequences according to the (a′). Thus, the vector of one or more embodiments of the present invention may comprise a combination of 2 or more sequences as set forth in the (a′), a combination of 2 or more sequences as set forth in the (b′), a combination of 2 or more sequences as set forth in the (c′), or a combination of 2 or more sequences as set forth in the (d′). Similarly, the vector of one or more embodiments of the present invention may comprise a combination of 2 or more sequences of the (e′). i.e., a nucleotide sequence as set forth in any of SEQ ID NOs: 39, 24, 27, 30, 33, 36, 42, 45, 48, 51, 54, 57, and 60, or equivalent sequences to any of these. Herein, the “equivalent sequence” to the sequences according to the (e′) means the sequences according to the (f′) to (h′) to the respective sequences according to the (e′). Thus, the vector of one or more embodiments of the present invention may comprise a combination of 2 or more sequences as set forth in the (e′), a combination of 2 or more sequences as set forth in the (f′), a combination of 2 or more sequences as set forth in the (g′), or a combination of 2 or more sequences as set forth in the (h′). The vector of one or more embodiments of the present invention may comprise, for example, a combination of 2 or more nucleotide sequences as set forth in any of SEQ ID NOs: 75, 65, and 67, or equivalent sequences to any of these. The vector of one or more embodiments of the present invention may comprise a combination of 2 or more nucleotide sequences as set forth in any of SEQ ID NOs: 39, 24, and 27, or equivalent sequences to any of these.

Further, one or more embodiments of the present invention also relate to a combination of 2 or more vectors comprising the nucleotide sequence as set forth in any of the (a) to (h) and (a′) to (h′). The number of combination and examples of the vector are the same as the combinations of the nucleotide sequences comprised in the vector, and hence the description is omitted.

The above nucleotide sequences comprised in the vector of one or more embodiments of the present invention were found by the comprehensive analysis on the nucleotide sequences of 4 chromosomal DNA of Komagataella pastoris (CBS7435 strain: ACCESSION No. FR839628 to FR839631 (J. Biotechnol. 154 (4), 312-320 (2011), and GS115 strain: ACCESSION No. FN392319 to FN392322 (Nat. Biotechnol. 27 (6), 561-566 (2009))). Specifically, the present inventors searched for a polypeptide comprising the amino acid sequence as set forth in SEQ ID NO: 93 or 94, and a polynucleotide comprising the nucleotide sequence as set forth in SEQ ID NO: 91 or 92 encoding the amino acid sequence. As a result, the inventor found the following 9 novel polypeptides having ACCESSION Nos. beginning with CCA in the CBS7435 strain, the following 4 novel polypeptides having ACCESSION Nos. beginning with CAY in the GS115 strain, and the following polynucleotides encoding these novel polypeptides:

a polypeptide under ACCESSION No. CCA36173 comprising the amino acid sequence as set forth in SEQ ID NO: 95, and a polynucleotide comprising the nucleotide sequence as set forth in SEQ ID NO: 24 encoding the polypeptide;

a polypeptide under ACCESSION No. CCA37695 comprising the amino acid sequence as set forth in SEQ ID NO: 96, and a polynucleotide comprising the nucleotide sequence as set forth in SEQ ID NOs: 27 encoding the polypeptide;

a polypeptide under ACCESSION No. CCA41161 comprising the amino acid sequence as set forth in SEQ ID NO: 97, and a polynucleotide comprising the nucleotide sequence as set forth in SEQ ID NO: 30 encoding the polypeptide;

a polypeptide under ACCESSION No. CCA41167 comprising the amino acid sequence as set forth in SEQ ID NO: 98, and a polynucleotide comprising the nucleotide sequence as set forth in SEQ ID NO: 33 encoding the polypeptide;

a polypeptide under ACCESSION No. CCA37701 comprising the amino acid sequence as set forth in SEQ ID NO: 99, and a polynucleotide comprising the nucleotide sequence as set forth in SEQ ID NO: 36 encoding the polypeptide;

a polypeptide under ACCESSION No. CCA40175 comprising the amino acid sequence as set forth in SEQ ID NO: 100, and a polynucleotide comprising the nucleotide sequence as set forth in SEQ ID NO: 39 encoding the polypeptide;

a polypeptide under ACCESSION No. CCA37509 comprising the amino acid sequence as set forth in SEQ ID NO: 101, and a polynucleotide comprising the nucleotide sequence as set forth in SEQ ID NO: 42 encoding the polypeptide;

a polypeptide under ACCESSION No. CCA38967 comprising the amino acid sequence as set forth in SEQ ID NO: 102, and a polynucleotide comprising the nucleotide sequence as set forth in SEQ ID NO: 45 encoding the polypeptide;

a polypeptide under ACCESSION No. CCA37504 comprising the amino acid sequence as set forth in SEQ ID NO: 103, and a polynucleotide comprising the nucleotide sequence as set forth in SEQ ID NO: 48 encoding the polypeptide:

a polypeptide under ACCESSION No. CAY67126 comprising the amino acid sequence as set forth in SEQ ID NO: 104, and a polynucleotide comprising the nucleotide sequence as set forth in SEQ ID NO: 51 encoding the polypeptide;

a polypeptide under ACCESSION No. CAY68445 comprising the amino acid sequence as set forth in SEQ ID NO: 105, and a polynucleotide comprising the nucleotide sequence as set forth in SEQ ID NO: 54 encoding the polypeptide;

a polypeptide under ACCESSION No. CAY68608 comprising the amino acid sequence as set forth in SEQ ID NO: 106, and a polynucleotide comprising the nucleotide sequence as set forth in SEQ ID NO: 57 encoding the polypeptide; and

a polypeptide under ACCESSION No. CAY71233 comprising the amino acid sequence as set forth in SEQ ID NO: 107, and a polynucleotide comprising the nucleotide sequence as set forth in SEQ ID NO: 60 encoding the polypeptide.

In Examples to be described later, the present inventors confirmed that a secretion amount of the protein is improved by introducing the vector comprising any of the above nucleotide sequences into a host and allowing the host to express the above polypeptide at a high level.

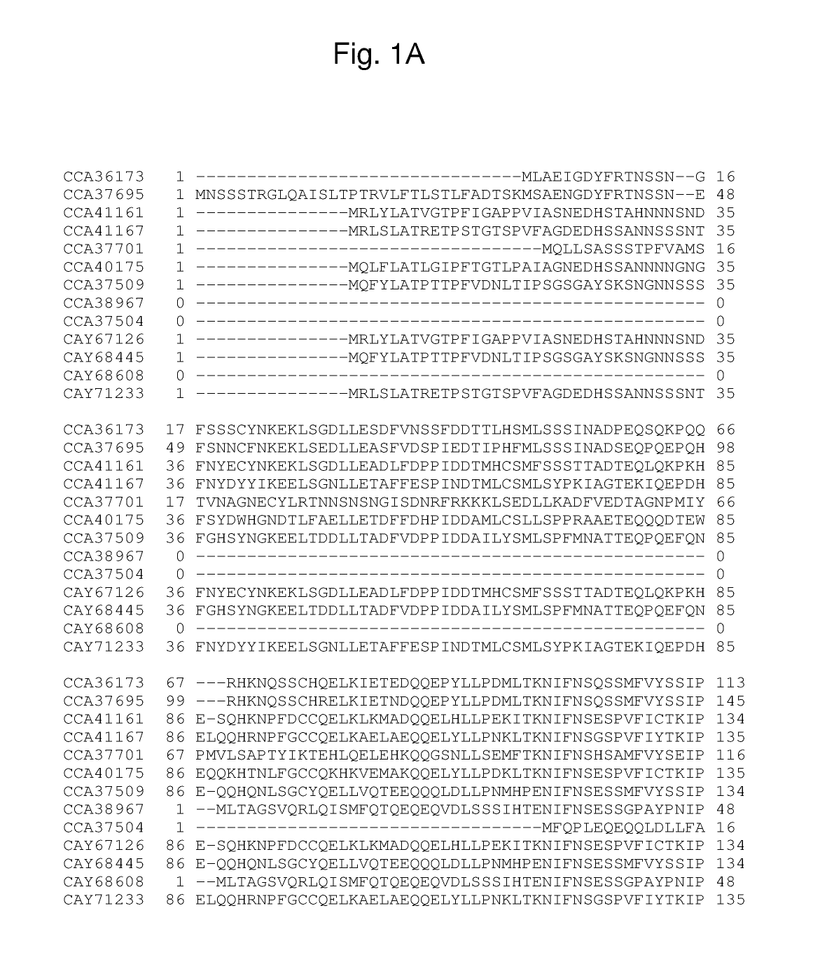

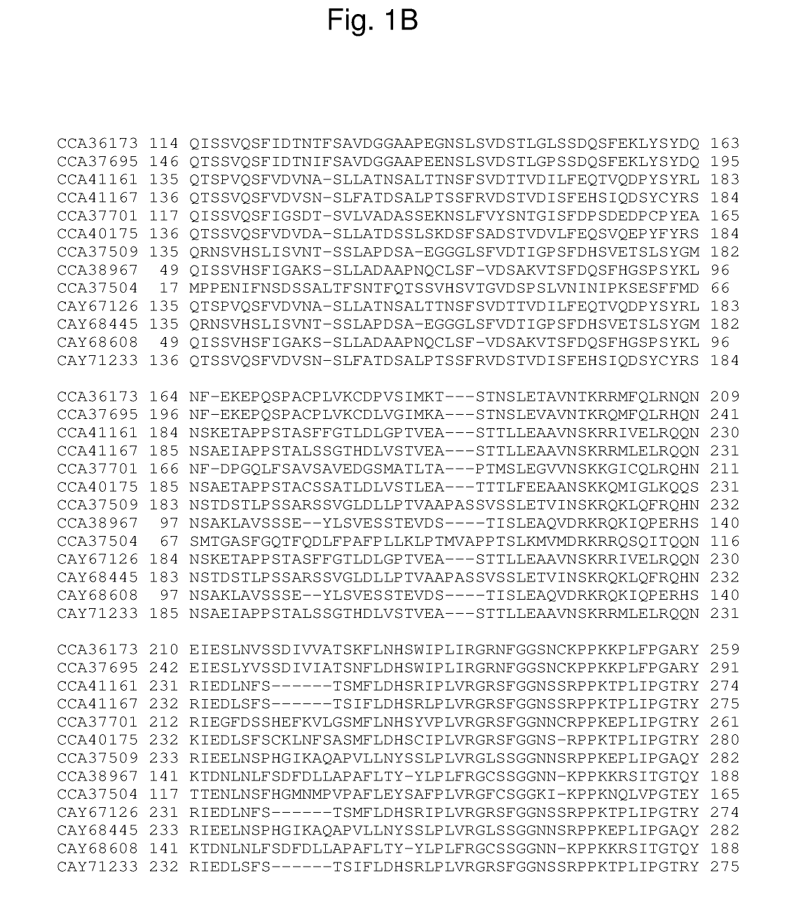

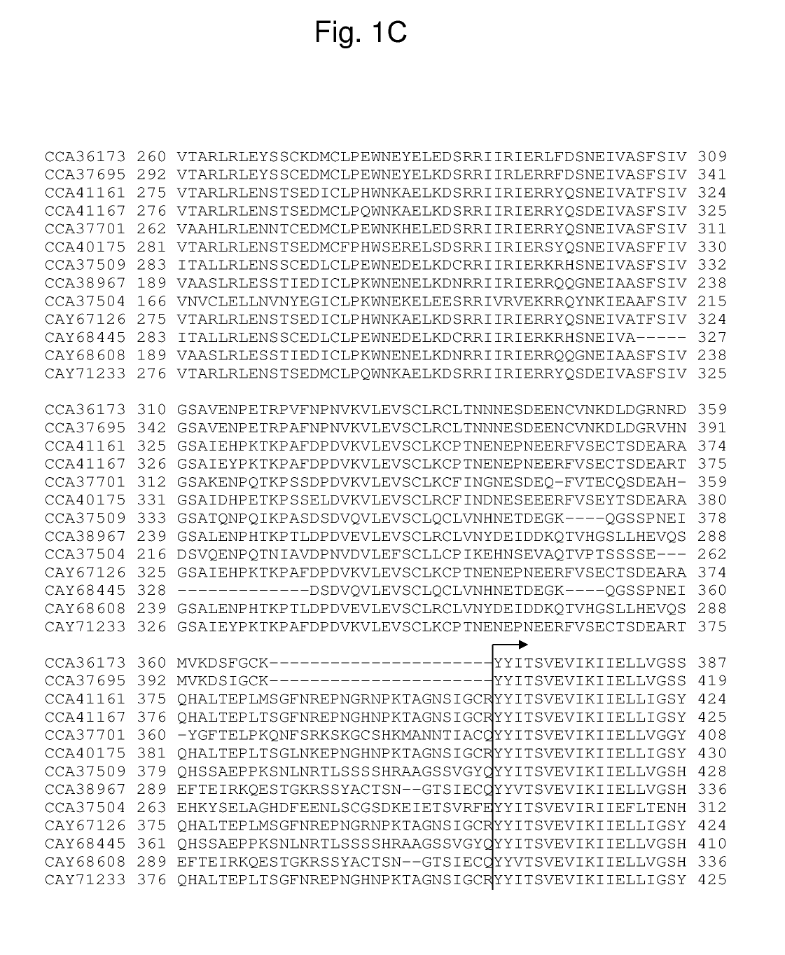

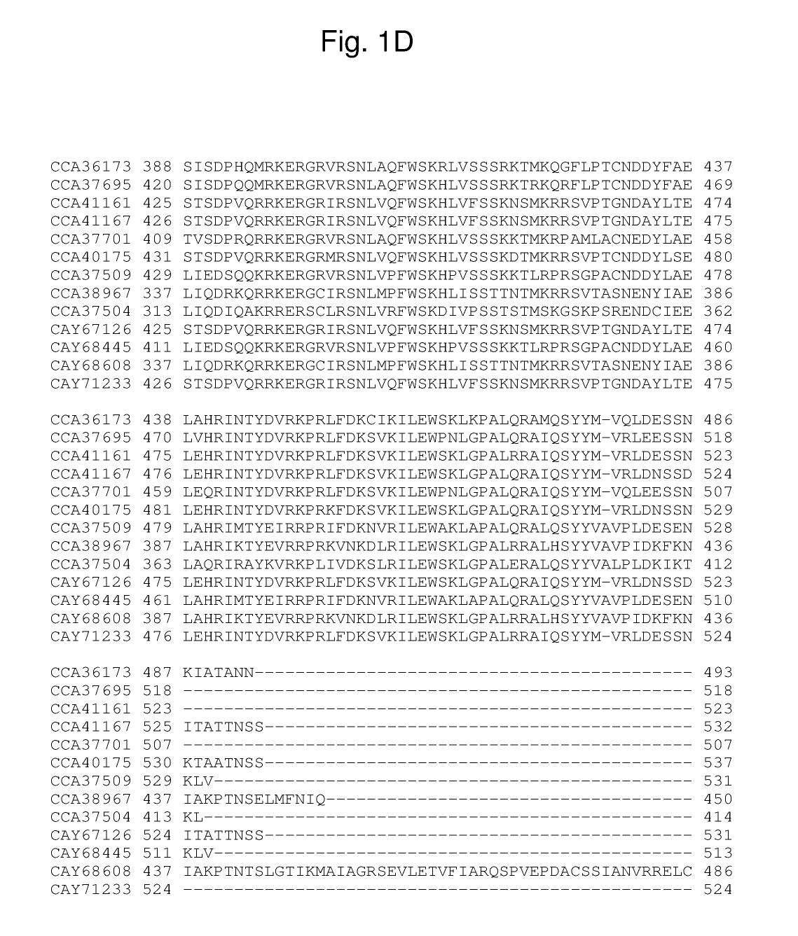

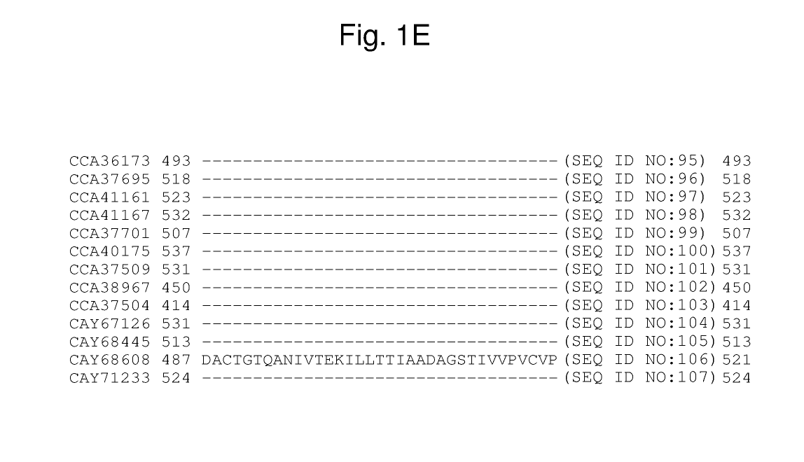

Additionally, the amino acid sequences of these novel polypeptides were aligned as shown in FIGS. 1A to 1E and deleting the C-terminal region at which a comparatively high identity was confirmed to prepare the following mutants:

a polypeptide comprising the amino acid sequence as set forth in any of SEQ ID NOs: 108 to 120 in which about 120 to about 200 amino acids are respectively deleted in the C-terminal region of the amino acid sequence as set forth in any of SEQ ID NOs: 95 to 107; and

a polynucleotide comprising the nucleotide sequence as set forth in any of SEQ ID NOs: 65, 67, 69, 71, 73, 75, 77, 79, 81, 83, 85, 87, and 89 respectively encoding the polypeptide.

In Examples to be described later, the present inventors introduced some of the vectors comprising any of the above nucleotide sequences into a host and allowing the host to express the above polypeptide in which the C-terminal region is deleted, thereby confirming that a secretion amount of the protein is improved but the degree of the improvement in secretion amount tends to be lowered compared to the case where the polypeptide comprising the C-terminal region is expressed. Thus, it is believed that, among the amino acids sequence as set forth in any of SEQ ID NOs: 95 to 107, the sequence at the N-terminal side improves the secretion amount of a protein while the sequence at the C-terminal side plays a role of enhancing the function of the sequence at the N-terminal side.

In one embodiment, the above vectors of one or more embodiments of the present invention can increase the secretion amount of a protein from a host cell, for example, when introduced to the host cell by transformation.

The “vector” in one or more embodiments of the present invention is a nucleic acid molecule constructed artificially, and may comprise, in addition to the above specified nucleotide sequence (referred to as “specified nucleotide sequence” to describe any of the above nucleotide sequences (a) to (h) or (a′) to (h′)), a cloning site comprising one or more restriction enzyme recognition sites, an overlapping region for utilizing an In-Fusion cloning system of Clontech Laboratories, Inc. or a Gibson Assembly system of New England Biolabs, a nucleotide sequence of an exogenous gene or an endogenous gene, a nucleotide sequence of a selectable marker gene (auxotrophic complementary gene, drug resistance gene). The vectors of one or more embodiments of the present invention may further comprise, depending on a host, autonomous replication sequence (ARS), centromere DNA sequence, or telomeric DNA sequence.

The above specified nucleotide sequence can be comprised in the vector while inserted in an expression cassette. The “expression cassette” refers to an expression system comprising the above specified nucleotide sequence and capable of providing the state to express it as a polypeptide. The “state to express” refers to a state where the above specified nucleotide sequence comprised in the expression cassette is arranged under the control of the elements required for gene expression in such a way as to be expressed in a transformant. Examples of the element required for gene expression include a promoter, a terminator, and the like. The vectors of one or more embodiments of the present invention can be a cyclic vector, a linear vector, or an artificial chromosome.

The “promotor” herein refers to a nucleotide sequence region located upstream of the above specified nucleotide sequence, wherein various transcription regulators relating to the promotion and repression of transcription, in addition to a RNA polymerase, bind to or work on the region to read the above specified nucleotide sequence which is a template, whereby a complimentary RNA is synthesized (transcribed).

For the promoter expressing a polypeptide, a promotor achieving the expression using a selected carbon source is suitably used and not particularly limited.

When the carbon source is methanol, the promoter for expressing a polypeptide includes AOX1 promoter, AOX2 promoter, CAT promoter, DHAS promoter, FDH promoter, FMD promoter, GAP promoter, and MOX promoter, but is not particularly limited thereto.

When the carbon source is glucose or glycerol, the promoter for expressing a polypeptide include GAP promoter, TEF promoter, LEU2 promoter, URA3 promoter, ADE promoter, ADH1 promoter, and PGK1 promoter, but is not particularly limited thereto.

The vector of one or more embodiments of the present invention is typically constituted by ligating a nucleic acid fragment comprising the above specified nucleotide sequence or a nucleic acid fragment consisting of the above specified nucleotide sequence to one or more other functional nucleic acid fragments as described above at both ends or one end thereof via, for example, a restriction enzyme recognition site.

The scope of vector according to one or more embodiments of the present invention encompasses, in addition to the form to which the above cloning site, overlapping region, nucleotide sequence of an exogenous gene or an endogenous gene, nucleotide sequence of a selectable marker gene, ARS, or centromere DNA sequence is added, nucleic acid molecules in the form to which these sequences can be added (for example, a form including a cloning site comprising one or more restriction enzyme recognition sites to which these sequences can be added).

The method for preparing the vector of one or more embodiments of the present invention is not particularly limited, but, for example, total synthesis, the PCR method, an In-Fusion cloning system of Clontech Laboratories, Inc. or a Gibson Assembly system of New England Biolabs can be used.

The method for introducing the vector into a host cell, i.e., the transformation method, can suitably be a known method, and examples include, when a yeast cell is used as a host, the electroporation method, the lithium acetate method, and the spheroplast method, but is not limited thereto. For example, the electroporation method described in High efficiency transformation by electroporation of Pichia pastoris pretreated with lithium acetate and dithiothreitol (Biotechniques. 2004 January; 36(1): 152-4.) is a common transformation method of Komagataella pastoris.

Herein, the increase in a secretion amount of a protein from a host cell to a secretion amount of a protein in the parent cell or the wild-type strain may be, for example, 1.01 times, 1.02 times, 1.03 times, 1.04 times, 1.05 times, 1.1 times, 1.2 times, 1.3 times, 1.4 times, 1.5 times, 1.6 times, 1.7 times, 1.8 times, 1.9 times, 2 times, 2.5 times, 3 times, 3.5 times, 4 times, 4.5 times, or 5 times or more, and may be 100 times, 90 times, 80 times, 70 times, 60 times, 50 times, 40 times, 30 times, 20 times, 10 times, 9 times, 8 times, 7 times, 6 times, or 5 times or less. The secretion amounts of all proteins from the cell can be easily determined using a cell culture supernatant by a method known by a person skilled in the art such as the Bladford method, the Lowry method, and the BCA method. The secretion amount of a specific protein from the cell can be easily determined using a cell culture supernatant by the ELISA method.

The “parent cell”, “wild-type strain”, or “host cell before the introduction” as used herein means a host cell or a strain which is not treated for changing the expression of a gene comprising the nucleotide sequence as set forth in any of the above (a) to (h) and (a′) to (h′). Thus, the “parent cell”, “wild-type strain”, or “host cell before the introduction” as used herein also includes a host cell or a strain in which a gene other than the gene comprising the nucleotide sequence as set forth in any of the above (a) to (h) and (a′) to (h′) is modified.

Herein, the “protein of interest” whose secretion is increased may be an endogenous protein of a host, or may be a heterologous protein or an exogenous protein. The “endogenous protein” as used herein refers to a protein produced during the culture of a host cell which is not genetically modified. On the contrary, the “heterologous protein” or the “exogenous protein” as used herein refers to a protein not typically expressed, or having an insufficient expression level or a secretion amount even when expressed in a host cell which is not genetically modified.

One or more embodiments of the present invention relate to a protein secretion enhancer consisting of the above vector. The protein secretion enhancer as used herein means a substance capable of increasing a secretion amount of a protein from a host cell when introduced into the host cell.

In one embodiment, one or more embodiments of the present invention relate to a composition for increasing a secretion amount of a protein in a host cell comprising the above vector or the protein secretion enhancer. The composition according to one or more embodiments of the present invention may contain an excipient, a carrier, a binder, a disintegrator, a buffer, and a solvent known by a person skilled in the art, in addition to the above vector or the protein secretion enhancer.

One or more embodiments of the present invention relate to a use of the vector in the enhancement of protein secretion.

The vector according to one or more embodiments of the present invention for expressing a polypeptide is useful in various uses such as host modification for the industrial purposes.

### 3. Mutant Cell and Cell Comprising Vector

One or more embodiments of the present invention relate to a mutant cell having an increased expression of a gene comprising the nucleotide sequence as set forth in any of the (a) to (h) and (a′) to (h′), and an increased secretion amount of a protein. The mutant cell of one or more embodiments of the present invention may have an increased expression of a combination of 2 or more genes comprising the nucleotide sequence as set forth in the (a) to (h). Examples of the number of combination include 3 or more, 4 or more, 5 or more, 6 or more, 7 or more, 8 or more, 9 or more, or 10 or more. For example, the mutant cell of one or more embodiments of the present invention may have an increased expression of a combination of 2 or more sequences of the (a), i.e., the nucleotide sequence encoding the amino acid sequence as set forth in any of SEQ ID NOs; 113, 108 to 112, and 114 to 120, or genes comprising an equivalent sequence to any of these. Thus, the mutant cell of one or more embodiments of the present invention may have an increased expression of a combination of 2 or more genes comprising the sequence as set forth in the (a), a combination of 2 or more genes comprising the sequence as set forth in the (b), a combination of 2 or more genes comprising the sequence as set forth in the (c), or a combination of 2 or more genes comprising the sequence as set forth in the (d). Similarly, the mutant cell of one or more embodiments of the present invention may have an increased expression of a combination of 2 or more sequences of the (e), i.e., a nucleotide sequence encoding the amino acid sequence as set forth in any of SEQ ID NOs: 100, 95 to 99, and 101 to 107, or genes comprising an equivalent sequence to any of these. Thus, the mutant cell of one or more embodiments of the present invention may have an increased expression of a combination of 2 or more genes comprising the sequence as set forth in the (e), a combination of 2 or more genes comprising the sequence as set forth in the (f), a combination of 2 or more genes comprising the sequence as set forth in the (g), or a combination of 2 or more genes comprising the sequence as set forth in the (h). The mutant cell of one or more embodiments of the present invention may have an increased expression of a combination of 2 or more nucleotide sequences encoding the amino acid sequence as set forth in any of SEQ ID NOs: 113, 108, and 109, or genes comprising an equivalent sequence to any of these. The mutant cell of one or more embodiments of the present invention may have an increased expression of a combination of 2 or more nucleotide sequences encoding the amino acid sequence as set forth in any of SEQ ID NOs: 100, 95, and 96, or genes comprising an equivalent sequence to any of these.

Further, the mutant cell of one or more embodiments of the present invention may have an increased expression of a combination of 2 or more genes comprising the nucleotide sequence as set forth in the (a′) to (h′). Examples of the number of combination include 3 or more, 4 or more, 5 or more, 6 or more, 7 or more, 8 or more, 9 or more, or 10 or more. For example, the mutant cell of one or more embodiments of the present invention may have an increased expression of a combination of 2 or more sequences of the (a′), i.e., the nucleotide sequence as set forth in any of SEQ ID NOs; 75, 65, 67, 69, 71, 73, 77, 79, 81, 83, 85, 87, and 89, or genes comprising an equivalent sequence to any of these. Thus, the mutant cell of one or more embodiments of the present invention may have an increased expression of a combination of 2 or more genes comprising the sequence as set forth in the (a′), a combination of 2 or more genes comprising the sequence as set forth in the (b′), a combination of 2 or more genes comprising the sequence as set forth in the (c′), or a combination of 2 or more genes comprising the sequence as set forth in the (d′). Similarly, the mutant cell of one or more embodiments of the present invention may have an increased expression of a combination of 2 or more sequences of the (e′), i.e., a nucleotide sequence as set forth in any of SEQ ID NOs: 39, 24, 27, 30, 33, 36, 42, 45, 48, 51, 54, 57, and 60, or genes comprising an equivalent sequence to any of these. Thus, the mutant cell of one or more embodiments of the present invention may have an increased expression of a combination of 2 or more genes comprising the sequence as set forth in the (e′), a combination of 2 or more genes comprising the sequence as set forth in the (f′), a combination of 2 or more genes comprising the sequence as set forth in the (g′), or a combination of 2 or more genes comprising the sequence as set forth in the (h′). The mutant cell of one or more embodiments of the present invention may have an increased expression of a combination of 2 or more nucleotide sequences as set forth in any of SEQ ID NOs: 75, 65, and 67, or genes comprising an equivalent sequence to any of these. The mutant cell of one or more embodiments of the present invention may have an increased expression of a combination of 2 or more nucleotide sequences as set forth in any of SEQ ID NOs; 39, 24, and 27, or genes comprising an equivalent sequence to any of these.

The mutant cell of one or more embodiments of the present invention has an increased expression of the above genes and an increased secretion amount of a protein compared with that of the parent cell or the wild-type strain.

The increase in an expression level of the gene compared with an expression level of the gene in the parent cell or the wild-type strain may be, for example, 1.01 times, 1.02 times, 1.03 times, 1.04 times, 1.05 times, 1.1 times, 1.2 times, 1.3 times, 1.4 times, 1.5 times, 1.6 times, 1.7 times, 1.8 times, 1.9 times, 2 times, 2.5 times, 3 times, 3.5 times, 4 times, 4.5 times, or 5 times or more, and may be 100 times, 90 times, 80 times, 70 times, 60 times, 50 times, 40 times, 30 times, 20 times, 10 times, 9 times, 8 times, 7 times, 6 times, or 5 times or less. The expression level of the gene can be easily determined using a method known by a person skilled in the art such as the RT-PCR method and the Real-time PCR method.

The mutant cell of one or more embodiments of the present invention can be obtained by a method known by a person skilled in the art, for example, treating using ultraviolet irradiation or a chemical mutagen such as N-methyl-N′-nitrosoguanidine and subsequently screening the cells having an increased expression level of genes comprising the nucleotide sequence as set forth in any of the (a) to (h) and (a′) to (h′). Further, the mutant cell of one or more embodiments of the present invention can also be obtained by transforming a host cell using the above vector. Furthermore, the mutant cell of one or more embodiments of the present invention can be obtained by disrupting or expressing at a high level of genes other than the gene comprising the nucleotide sequence as set forth in any of the (a) to (h) and (a′) to (h′), and screening the cells having an increased expression level of genes comprising the nucleotide sequence as set forth in any of the (a) to (h) and (a′) to (h′).

One or more embodiments of the present invention relate to a cell comprising the vector according to the above section 2. The cell according to one or more embodiments of the present invention may be, for example, a transformant obtained by transforming a host cell by using the above vector. The transformation method is as described in the above section 2.

After performing transformation, the selectable marker for selecting the transformant is not particularly limited. For example, when a yeast is used as a host cell, a vector comprising an auxotrophic complementary gene such as URA3 gene, LEU2 gene, ADE1 gene, HIS4 gene, or ARG4 gene is used as the selectable marker gene, and transformation is carried out using auxotrophic strains of uracil, leucine, adenine, histidine, and arginine, respectively as the host cell, and then the transformant can be selected by the recovery of prototrophic phenotype. Alternatively, when a vector comprising a drug resistance gene such as a G418 resistance gene, a Zeocin (tradename) resistance gene, or a hyglomycin resistance gene as the selectable marker gene is used, the transformant can be selected by the resistance on the medium containing G418, Zeocin (tradename), and hyglomycin, respectively. The auxotrophic selectable marker used for preparing a yeast host cannot be used when such a selectable marker in the host is not disrupted. In this instance, the selectable marker may be disrupted in the host, and a method known by a person skilled in the art can be used.

The cell according to one or more embodiments of the present invention may be a transformant obtained by transforming the vector, or a progenic cell of such a transformant. The number of copies of the vector to be introduced into a single cell of the transformant is not particularly limited. A cell may comprise 1 copy or 2 or more multiple copies of vectors per cell. One copy of vector may exist as a cyclic vector, a linear vector, or an artificial chromosome, or may be incorporated into a chromosome derived from the host. Two or more multiple copies of vectors may all exist as cyclic vectors, linear vectors, or artificial chromosomes, or may all be incorporated into a chromosome derived from the host, or both states may occur simultaneously. The 2 or more multiple copies of vectors may be multiple copies of vectors having 2 or more copies of the same vector, or may be multiple copies of vectors having one or more copies of different vectors. For examples, the cell according to one or more embodiments of the present invention may have in combination of 2 or more different vectors comprising the nucleotide sequence as set forth in any of the (a) to (h) and (a′) to (h′), and may thereby have an increased expression of genes comprising the nucleotide sequence as set forth in any of the (a) to (h) and (a′) to (h′) in combination as described above.

One or more embodiments of the present invention relate to a method for preparing a mutant strain comprising a step of introducing the vector or the protein secretion enhancer described in the above section 2 into a host cell, wherein the mutant strain has an increased secretion amount of a protein compared to that of the host cell before the introduction.

### 4. Method for Producing Protein of Interest

One or more embodiments of the present invention relate to a method for producing a protein of interest comprising a step of culturing the cell described in the above section 3, and a step of recovering the protein of interest from a culture medium.

The “culture medium” as used herein means, in addition to a culture supernatant, a cultured cell, a cultured cell body, or a lysate of cell or cell body. Thus, the method for producing a protein of interest using the transformed yeast of one or more embodiments of the present invention includes a method comprising culturing the cell described in the above section 3 and allowing the protein to accumulate in the cell body thereof or the culture supernatant, for example in the culture supernatant.

The cell culture conditions are not particularly limited and suitably selected depending on the cell. In the culture, any medium containing a nutrition source utilized by the cell can be used. Usable is a typical medium containing, as the nutrition source: a carbon source such as saccharides such as glucose, sucrose and maltose, organic acids such as lactic acid, acetic acid, citric acid, and propionic acid, alcohols such as methanol, ethanol, and glycerol, hydrocarbons such as a paraffin, oils such as a soybean oil and a rapeseed oil, or a mixture thereof: a nitrogen source such as ammonium sulfate, ammonium phosphate, urea, a yeast extract, a meat extract, peptone, and a corn steep liquor: and further nutrition sources such as other inorganic salts and vitamins, suitably mixed and added thereto. Additionally, the culture can be carried out by either batch culture or continuous culture.

In one or more embodiments of the present invention, the above carbon source may be 1 or 2 or more of glucose, glycerol, and methanol when a yeast belonging to the genus Komagataella or a yeast belonging to the genus Ogataea is used as the yeast. Additionally, these carbon sources may be present from the beginning of culture or may be added during the culture.

The “protein of interest” is a protein produced by the cell into which the vector of one or more embodiments of the present invention is introduced, and may be an endogenous protein of the host, or a heterologous protein. Examples of the protein of interest include enzymes derived from microorganisms, proteins produced by animals and plants which are multicellular organisms. Examples include phytase, protein A, protein G, protein L, amylase, glucosidase, cellulase, lipase, protease, glutaminase, peptidase, nuclease, oxidase, lactase, xylanase, trypsin, pectinase, isomerase, and fluorescent proteins, but are not limited thereto. Particularly, proteins for treating human and/or animals may be used.

Examples of the protein for treating human and/or animals specifically include a hepatitis B virus surface antigen, hirudin, an antibody, a human antibody, a partial antibody, a human partial antibody, serum albumin, human serum albumin, an epidermal growth factor, a human epidermal growth factor, insulin, a growth hormone, erythropoietin, interferon, blood coagulation factor VIII, granulocyte-colony stimulating factor (G-CSF), granulocyte-macrophage colony stimulating factor (GM-CSF), thrombopoietin, IL-1, IL-6, tissue plasminogen activator (TPA), urokinase, leptin, and stem cell factor (SCF).

The “antibody” used herein refers to a heterotetramer protein constituted by polypeptide chains of 2 of each the L chain and H chain being disulfide bonded, and is not particularly limited as long as it has an binding ability to a specific antigen.

The “partial antibody” used herein refers to Fab antibody, (Fab)2 antibody, scFv antibody, diabody antibody, and derivatives thereof, and is not particularly limited as long as it has a binding ability to a specific antigen. The Fab antibody refers to a heteromer protein wherein the L chain and the Fd chain of the antibody are bound by an S—S bond, or a heteromer protein wherein the L chain and the Fd chain of the antibody associate without comprising an S—S bond, and is not particularly limited as long as it has an binding ability to a specific antigen.

The amino acid constituting the above protein of interest may be naturally occurred, non-naturally occurred, or modified. Additionally, the amino acid sequence of the protein may be artificially modified or de-novo designed.

A protein of interest is allowed to accumulate in a host or culture medium by culturing the transformant obtained by introducing the vector of one or more embodiments of the present invention into the cell, and can be recovered. For the method for recovering a protein of interest, known purification methods can be used in a suitable combination. For example, the transformed yeast is cultured in a suitable medium, and the cell body are removed from the culture supernatant by centrifugation of the culture medium or filtration treatment. A protein of interest is recovered from the culture supernatant by subjecting the obtained culture supernatant to a process singly or in combination such as salting out (ammonium sulfate precipitation or sodium phosphate precipitation), solvent precipitation (protein fractional precipitation such as acetone or ethanol), dialysis, gel filtration chromatography, ion exchange chromatography, hydrophobic chromatography, affinity chromatography, reversed phase chromatography, or ultrafiltration.

The cell culture can be typically carried out under general conditions, and, for examples, in the case of a yeast, can be carried out by aerobically culturing at pH 2.5 to 10.0 in a temperature range from 10° C. to 48° C. for 10 hours to 10 days.

The recovered protein of interest can be used directly, but can also be used after adding a modification for causing a pharmacological change such as PEGylation or a modification for adding a function such as that of an enzyme or an isotope. Additionally, various formulation treatments may be used.

To secrete a non-secretory protein of interest outside the cell body, a nucleotide sequence encoding a signal sequence may be introduced to the 5′-end of the protein of interest gene. The nucleotide sequence encoding a signal sequence is not particularly limited as long as a nucleotide sequence encodes a signal sequence by which the yeast secretory expression is achieved, but examples include Mating Factor α (MF α) of Saccharomyces cerevisiae, acid phosphatase of Ogataea angusta (PHO1), acid phosphatase of Komagataella pastoris (PHO1), invertase of Saccharomyces cerevisiae (SUC2), PLB1 of Saccharomyces cerevisiae, bovine serum albumin (BSA), human serum albumin (HSA), and a nucleotide sequence encoding a signal sequence of an immunoglobulin.

The promoter for a protein of interest in one or more embodiments of the present invention is not particularly limited as long as the promoter expresses the protein of interest, for example at a high level of the protein of interest, in the transformant. A methanol-inducible promoter may be used. The methanol-inducible promoter is not particularly limited as long as a promoter has a transcriptional activity when the carbon source is methanol, and examples include AOX1 promoter, AOX2 promoter, CAT promoter, DHAS promoter, FDH promoter, FMD promoter, GAP promoter, and MOX promoter.

The expression at a high level of a polypeptide can be achieved by a known technique such as, in addition to the expression induction of a nucleotide sequence encoding the polypeptide on the chromosome and/or the vector, a modification of a nucleotide sequence encoding a polypeptide on the chromosome and/or the vector (increase in the number of copies, insertion of a promoter, or codon modification), introduction of a nucleotide sequence encoding the polypeptide into a host cell, or obtaining a strain expressing polypeptide at a high level by introducing mutation(s) into a host cell.

The modification of a nucleotide sequence encoding a polypeptide on the chromosome can be carried out using a technique such as gene insertion utilizing the homologous recombination between the host chromosomes or site-specific mutagenesis. For example, such a modification can be carried out by introducing a nucleotide sequence encoding the polypeptide into a chromosome for increasing the number of copies, substituting the promoter with a much stronger promoter, or modifying a nucleotide sequence encoding a polypeptide to a codon suitable for a host cell.

Hereinafter, one or more embodiments of the present invention are described in detail in reference to Examples, but the present invention shall not be limited thereto.

### EXAMPLES

### Example 1: Preparation of Various Genes Used for Preparing Vectors

A detailed engineering method and the like related to recombinant DNA technology used in the Examples below are described in the following books: Molecular Cloning 2nd Edition (Cold Spring Harbor Laboratory Press, 1989) and Current Protocols in Molecular Biology (Greene Publishing Associates and Wiley-Interscience).

In the Examples below, a plasmid used for transforming a yeast was prepared by introducing a constructed vector into an Escherichia coli (E. coli) DH5a competent cell (from Takara Bio Inc.) and amplifying a resulting transformant by culturing the transformant. Preparation of the plasmid from the strain carrying the plasmid was performed by using QIAprep spin miniprep kit (from QIAGEN).

An AOX1 promoter (SEQ ID NO: 2), an AOX1 terminator (SEQ ID NO: 5), an HIS4 sequence (SEQ ID NO: 8), a GAP promoter (SEQ ID NO: 15), a nucleotide sequence encoding a polypeptide represented by ACCESSION No. CCA36173 (SEQ ID NO: 24), a nucleotide sequence encoding a polypeptide represented by ACCESSION No. CCA37695 (SEQ ID NO: 27), a nucleotide sequence encoding a polypeptide represented by ACCESSION No. CCA41161 (SEQ ID NO: 30), a nucleotide sequence encoding a polypeptide represented by ACCESSION No. CCA41167 (SEQ ID NO: 33), a nucleotide sequence encoding a polypeptide represented by ACCESSION No. CCA37701 (SEQ ID NO: 36), a nucleotide sequence encoding a polypeptide represented by ACCESSION No. CCA40175 (SEQ ID NO: 39), a nucleotide sequence encoding a polypeptide represented by ACCESSION No. CCA37509 (SEQ ID NO: 42), a nucleotide sequence encoding a polypeptide represented by ACCESSION No. CCA38967 (SEQ ID NO: 45), a nucleotide sequence encoding a polypeptide represented by ACCESSION No. CCA37504 (SEQ ID NO: 48), and a CCA38473 terminator (SEQ ID NO: 21) were used for vector construction. These sequences were prepared by PCR using as a template a mixture of chromosomal DNA from Komagataella pastoris strain ATCC76273 (the nucleotide sequence thereof is described in EMBL (The European Molecular Biology Laboratory) ACCESSION No. FR839628 to FR839631). The AOX1 promoter was prepared by PCR using primer 1 (SEQ ID NO: 3) and primer 2 (SEQ ID NO: 4). The AOX1 terminator was prepared by PCR using primer 3 (SEQ ID NO: 6) and primer 4 (SEQ ID NO: 7). The HIS4 sequence was prepared by PCR using primer 5 (SEQ ID NO: 9) and primer 6 (SEQ ID NO: 10). The GAP promoter was prepared by PCR using primer 9 (SEQ ID NO: 16) and primer 10 (SEQ ID NO: 17). The CCA38473 terminator was prepared by PCR using primer 13 (SEQ ID NO: 22) and primer 14 (SEQ ID NO: 23).

A Zeocin (TM)-resistance gene under promoter control (SEQ ID NO: 18), which was used for vector construction, was prepared by PCR using synthetic DNA as a template. An anti-β galactosidase single-chain antibody gene having a Mating Factorα pre-pro signal sequence added thereto (SEQ ID NO: 11), which was used for vector construction, was prepared by PCR using synthetic DNA as a template, based on a published sequence database (J Mol Biol. 1998 Jul. 3; 280(1):117-27.). A nucleotide sequence encoding a polypeptide represented by ACCESSION No. CAY67126 (SEQ ID NO: 51), a nucleotide sequence encoding a polypeptide represented by ACCESSION No. CAY68445 (SEQ ID NO: 54), a nucleotide sequence encoding a polypeptide represented by ACCESSION No. CAY68608 (SEQ ID NO: 57), and a nucleotide sequence encoding a polypeptide represented by ACCESSION No. CAY71233 (SEQ ID NO: 60), which were used for vector construction, were prepared by PCR using synthetic DNA as a template.

PCR was performed by using Prime STAR HS DNA Polymerase (from Takara Bio Inc.) etc. under a reaction condition described in the accompanying manual. Preparation of the chromosomal DNA from Komagataella pastoris strain ATCC76273 was performed by using Dr. GenTLE™ (from Takara Bio Inc.) etc. under the condition described therein.

### Example 2: Construction of Anti-β Galactosidase Single-Chain Antibody Expression Vector

A gene fragment having a multiple cloning site. HindIII-BamHI-BglII-XbaI-EcoRI, (SEQ ID NO: 1) was totally synthesized and this was inserted into a HindIII-EcoRI site of pUC19 (from Takara Bio Inc., Code No. 3219), thereby constructing pUC-1.

Furthermore, a nucleic acid fragment in which BamHI recognition sequences were added to both sides of an AOX1 promoter (SEQ ID NO: 2) was prepared by PCR using primer 1 (SEQ ID NO: 3) and primer 2 (SEQ ID NO: 4) and inserted into the BamHI site of pUC-1 after treatment with BamHI, thereby constructing pUC-Paox.

Then, a nucleic acid fragment in which XbaI recognition sequences were added to both sides of an AOX1 terminator (SEQ ID NO: 5) was prepared by PCR using primer 3 (SEQ ID NO: 6) and primer 4 (SEQ ID NO: 7) and inserted into the XbaI site of pUC-Paox after treatment with XbaI, thereby constructing pUC-PaoxTaox.

Then, a nucleic acid fragment in which EcoRI recognition sequences were added to both sides of an HIS4 sequence (SEQ ID NO: 8) was prepared by PCR using primer 5 (SEQ ID NO: 9) and primer 6 (SEQ ID NO: 10) and inserted into the EcoRI site of pUC-PaoxTaox after treatment with EcoRI, thereby constructing pUC-PaoxTaoxHIS4.

Then, a nucleic acid fragment in which BglII recognition sequences were added to both sides of an anti-β galactosidase single-chain antibody gene having a Mating Factorα pre-pro signal sequence added thereto (SEQ ID NO: 11) was prepared by PCR using primer 7 (SEQ ID NO: 12) and primer 8 (SEQ ID NO: 13) and inserted into the BglII site of pUC-PaoxTaoxHIS4 after treatment with BglII, thereby constructing pUC-PaoxscFvTaoxHIS4. This pUC-PaoxscFvTaoxHIS4 is designed to express the anti-β galactosidase single-chain antibody under the control of the AOX1 promoter.

### Example 3: Construction of Polypeptide Expression Vectors

A gene fragment having a multiple cloning site, HindIII-BamHI-SpeI-XbaI-EcoRI, (SEQ ID NO: 14) was totally synthesized and this was inserted into a HindIII-EcoRI site of pUC19 (from Takara Bio Inc., Code No. 3219), thereby constructing pUC-2.

Furthermore, a nucleic acid fragment in which BamHI recognition sequences were added to both sides of a GAP promoter (SEQ ID NO: 15) was prepared by PCR using primer 9 (SEQ ID NO: 16) and primer 10 (SEQ ID NO: 17) and inserted into the BamHI site of pUC-2 after treatment with BamHI, thereby constructing pUC-Pgap.

Then, a nucleic acid fragment in which EcoRI recognition sequences were added to both sides of a Zeocin (TM)-resistance gene under promoter control (SEQ ID NO: 18) was prepared by PCR using primer 11 (SEQ ID NO: 19) and primer 12 (SEQ ID NO: 20) and inserted into the EcoRI site of pUC-Pgap after treatment with EcoRI, thereby constructing pUC-PgapZeo.

Then, a nucleic acid fragment in which XbaI recognition sequences were added to both sides of a CCA38473 terminator (SEQ ID NO: 21) was prepared by PCR using primer 13 (SEQ ID NO: 22) and primer 14 (SEQ ID NO: 23) and inserted into the XbaI site of pUC-PgapZeo after treatment with XbaI, thereby constructing pUC-PgapT38473Zeo.

Then, nucleotide sequences encoding novel polypeptides listed in Table 1 were prepared by PCR using a mixture of chromosomal DNA from Komagataella pastoris strain ATCC76273 (the nucleotide sequences thereof are described under in EMBL (The European Molecular Biology Laboratory) ACCESSION No. FR839628 to FR839631) or synthetic DNA as a template. The ACCESSION Nos. of the novel polypeptides, amino acid sequences of the novel polypeptides, nucleotide sequences encoding the novel polypeptides, the numbers and sequences of the primers (Fw and Rev) used for amplification of the nucleic acid fragments, and the templates are shown in Table 1 below.

TABLE 1

| | | | Number and | Number and | |
|---|---|---|---|---|---|
| ACCESSION | Amino acid | Nucleotide | sequence of | sequence of | |
| NO. | sequence | sequence | primers (Fw) | primers (Rev) | Template |
| CCA36173 | SEQ ID NO: 95 | SEQ ID NO: 24 | 15 (SEQ ID NO: 25) | 16 (SEQ ID NO: 26) | Chromosomal DNA |
| | | | | | of strain ATCC76273 |
| CCA37695 | SEQ ID NO: 96 | SEQ ID NO: 27 | 17 (SEQ ID NO: 28) | 18 (SEQ ID NO: 29) | Chromosomal DNA |
| | | | | | of strain ATCC76273 |
| CCA41161 | SEQ ID NO: 97 | SEQ ID NO: 30 | 19 (SEQ ID NO: 31) | 20 (SEQ ID NO: 32) | Chromosomal DNA |
| | | | | | of strain ATCC76273 |
| CCA41167 | SEQ ID NO: 98 | SEQ ID NO: 33 | 21 (SEQ ID NO: 34) | 22 (SEQ ID NO: 35) | Chromosomal DNA |
| | | | | | of strain ATCC76273 |
| CCA37701 | SEQ ID NO: 99 | SEQ ID NO: 36 | 23 (SEQ ID NO: 31) | 24 (SEQ ID NO: 38) | Chromosomal DNA |
| | | | | | of strain ATCC76273 |
| CCA40175 | SEQ ID NO: 100 | SEQ ID NO: 39 | 25 (SEQ ID NO: 40) | 26 (SEQ ID NO: 41) | Chromosomal DNA |
| | | | | | of strain ATCC76273 |
| CCA37509 | SEQ ID NO: 101 | SEQ ID NO: 42 | 27 (SEQ ID NO: 43) | 28 (SEQ ID NO: 44) | Chromosomal DNA |
| | | | | | of strain ATCC76273 |
| CCA38967 | SEQ ID NO: 102 | SEQ ID NO: 45 | 29 (SEQ ID NO: 46) | 30 (SEQ ID NO: 47) | Chromosomal DNA |
| | | | | | of strain ATCC76273 |
| CCA37504 | SEQ ID NO: 103 | SEQ ID NO: 48 | 31 (SEQ ID NO: 49) | 32 (SEQ ID NO: 50) | Chromosomal DNA |
| | | | | | of strain ATCC76273 |
| CAY67126 | SEQ ID NO: 104 | SEQ ID NO: 51 | 33 (SEQ ID NO: 52) | 34 (SEQ ID NO: 53) | Synthetic DNA |
| CAY68445 | SEQ ID NO: 105 | SEQ ID NO: 54 | 35 (SEQ ID NO: 55) | 36 (SEQ ID NO: 56) | Synthetic DNA |
| CAY68608 | SEQ ID NO: 106 | SEQ ID NO: 57 | 37 (SEQ ID NO: 58) | 38 (SEQ ID NO: 59) | Synthetic DNA |
| CAY71233 | SEQ ID NO: 107 | SEQ ID NO: 60 | 39 (SEQ ID NO: 61) | 40 (SEQ ID NO: 62) | Synthetic DNA |

In each of these nucleic acid fragments, the GAP promoter sequence is added, as an overlapping region, upstream of the nucleotide sequence encoding the novel polypeptide and the CCA38473 terminator sequence is added, as an overlapping region, downstream of the nucleotide sequence encoding the novel polypeptide.

After treating pUC-PgapT38473Zeo with SpeI, a nucleic acid fragment was prepared by PCR using a Re primer for the GAP promoter (primer 41 (SEQ ID NO: 63)) and a Fw primer for the CCA38473 terminator (primer 42 (SEQ ID NO: 64)). This nucleic acid fragment was mixed with the nucleic acid fragment prepared by PCR that has the above-mentioned nucleotide sequence encoding the novel polypeptide. Then, these fragments were linked together by using a Gibson Assembly system from New England Biolabs Inc., thereby constructing pUC-PgapCCA36173T38473Zeo, pUC-PgapCCA37695T38473Zeo, pUC-PgapCCA41161T38473Zeo, pUC-PgapCCA41167T38473Zeo, pUC-PgapCCA37701T38473Zeo, pUC-PgapCCA40175T38473Zeo, pUC-PgapCCA37509T38473Zeo, pUC-PgapCCA38967T38473Zeo, pUC-PgapCCA37504T38473Zeo, pUC-PgapCAY67126T38473Zeo, pUC-PgapCAY68445T38473Zeo, pUC-PgapCAY68608T38473Zeo, or pUC-PgapCAY71233T38473Zeo. These vectors are designed to express each polypeptide under the control of the GAP promoter.

### Example 4: Generation of Transformed Yeast

The anti-β galactosidase single-chain antibody expression vector pUC-PaoxscFvTaoxHIS4 constructed in Example 2 and the polypeptide expression vectors constructed in Example 3 were used to transform Komagataella pastoris as described below.

A histidine auxotrophic strain derived from Komagataella pastoris strain ATCC76273 was inoculated in 3 ml of YPD medium (1% yeast extract bacto (from Becton, Dickinson and Company), 2% polypeptone (from NIHON PHARMACEUTICAL CO., LTD.), 2% glucose) and cultured with shaking overnight at 30° C. to obtain a preculture medium. 500 μl of the preculture medium thus obtained was inoculated in 50 ml of YPD medium and cultured with shaking up to an OD600 of 1 to 1.5. Then, the yeast was harvested (3000×g, 10 minutes, 20° C.) and resuspended in 10 ml of 50 mM potassium phosphate buffer, pH 7.5 supplemented with 250 μl of 1M DTT (final concentration of 25 mM).

After incubating this suspension for 15 minutes at 30° C., the yeast was harvested (3000×g, 10 minutes, 20° C.) and washed with 50 ml of STM buffer precooled in ice (270 mM sucrose, 10 mM Tris-HCl, 1 mM magnesium chloride, pH7.5). After harvesting the yeast from the washing solution (3000×g, 10 minutes, 4° C.) and washing again with 25 ml of STM buffer, the yeast was harvested (3000×g, 10 minutes, 4° C.). Finally, the obtained yeast was suspended in 250 μl of the ice cold STM buffer and this suspension was used as a competent cell solution.

E. coli was transformed by using the anti-1 galactosidase single-chain antibody expression vector pUC-PaoxscFvTaoxHIS4 constructed in Example 2 and the transformant obtained was cultured in 5 ml of 2YT medium containing ampicillin (1.6% tryptone bacto (from Becton, Dickinson and Company), 1% yeast extract bacto (from Becton, Dickinson and Company), 0.5% sodium chloride, 0.01% ampicillin sodium (from FUJIFILM Wako Pure Chemical Corporation)). pUC-PaoxscFvTaoxHIS4 was obtained from the resulting cells by using a QIAprep spin miniprep kit (from QIAGEN). This plasmid was linearized with SacI treatment by utilizing a SacI recognition sequence within an AOX1 promoter.

60 μl of the above-mentioned competent cell solution and 1 μl of solution of the linearized pUC-PaoxscFvTaoxHIS4 were mixed and transferred into an electroporation cuvette (disposable cuvette electrode, electrode gap of 2 mm (from BM Equipment Co., Ltd.)). After the mixture was subjected to electroporation at 7.5 kV/cm, 25 μF, and 200Ω, cell bodies were suspended in 1 ml of YPD medium and allowed to stand for one hour at 30° C. After allowing to stand for one hour, the yeast was harvested (3000×g, 5 minutes, 20° C.) and suspended in 1 ml of YNB medium (0.67% yeast nitrogen base Without Amino Acid (from Becton, Dickinson and Company). Then, yeast was harvested again (3000×g, 5 minutes, 20° C.). The cell bodies were resuspended in an adequate amount of YNB medium and then spread on a selective YNB agar plate (0.67% yeast nitrogen base Without Amino Acid (from Becton, Dickinson and Company), 1.5% agarose, 2% glucose). A strain that grew in static culture for 3 days at 30° C. was selected to obtain a yeast expressing the anti-β galactosidase single-chain antibody.

Subsequently, Komagataella pastoris strain ATCC76273 and the yeast expressing the anti-β galactosidase single-chain antibody were inoculated individually in 3 ml of YPD medium (1% yeast extract bacto (from Becton, Dickinson and Company), 2% polypeptone (from NIHON PHARMACEUTICAL CO., LTD.), 2% glucose) and cultured with shaking overnight at 30° C. to obtain a preculture medium. 500 μl of the preculture medium thus obtained was inoculated in 50 ml of YPD medium and cultured with shaking up to an OD600 of 1 to 1.5. Then, The yeast was harvested (3000×g, 10 minutes, 20° C.) and resuspended in 10 ml of 50 mM potassium phosphate buffer, pH 7.5 supplemented with 250 μl of 1M DTT (final concentration of 25 mM).

After incubating this suspension for 15 minutes at 30° C., the yeast was harvested (3000×g, 10 minutes, 20° C.) and washed with 50 ml of STM buffer precooled in ice (270 mM sucrose, 10 mM Tris-HCl, 1 mM magnesium chloride, pH7.5). After harvesting the yeast from the washing solution (3000×g, 10 minutes, 4° C.) and washing again with 25 ml of STM buffer, the yeast was harvested (3000×g, 10 minutes, 4° C.). Finally, the obtained yeast was suspended in 250 μl of the ice cold STM buffer and this suspension was used as a competent cell solution.

E. coli was transformed by using the polypeptide expression vectors constructed in Example 3 and the obtained transformant was cultured in 5 ml of 2YT medium containing ampicillin (1.6% tryptone bacto (from Becton, Dickinson and Company), 1% yeast extract bacto (from Becton, Dickinson and Company), 0.5% sodium chloride, 0.01% ampicillin sodium (from FUJIFILM Wako Pure Chemical Corporation)). Each polypeptide expression vector was obtained from the resulting cell bodies by using a QIAprep spin miniprep kit (from QIAGEN). This plasmid was linearized with NruI treatment by utilizing an NruI recognition sequence within a CCA38473 terminator.

60 μl of the above-mentioned competent cell solution and 1 μl of solution of the linearized polypeptide expression vector were mixed and transferred into the electroporation cuvette (disposable cuvette electrode, electrode gap of 2 mm (from BM Equipment Co., Ltd.)). After the mixture was subjected to electroporation at 7.5 kV/cm, 25 μF, and 200Ω, cell bodies were suspended in 1 ml of YPD medium and allowed to stand for one hour at 30° C. After allowing to stand for one hour, the yeast was harvested (3000×g, 5 minutes, 20° C.) and 961 μl of the supernatant was discarded. The yeast was resuspended in 100 μl of the remaining solution and then 100 μl of the suspension was spread on a selective YPDZeocin™ agar plate (1% yeast extract bacto (from Becton, Dickinson and Company), 2% polypeptone (from NIHON PHARMACEUTICAL CO., LTD.), 2% glucose, 1.5% agarose, 0.01% Zeocin™ (from Thermo Fisher Scientific Inc.). A strain that grew in static culture for 3 days at 30° C. was selected to obtain a yeast expressing the polypeptide and a yeast expressing both the anti-3 galactosidase single-chain antibody and the polypeptide.

### Example 5: Construction of Expression Vector of c-Terminally Deleted Polypeptide

A nucleotide sequence encoding a polypeptide, in which C-terminal was deleted, was prepared by PCR using the polypeptide expression vector constructed in Example 3 as a template. The alignment of amino acid sequences of novel polypeptides and deleted C-terminal regions having relatively high homology to one another are shown in FIGS. 1A to 1E. The name of the C-terminally deleted polypeptides, the amino acid sequences of the polypeptides, the nucleotide sequences encoding the polypeptides, the sequences of primers used for amplification of nucleic acid fragments, and the templates are shown in Table 2 below.

TABLE 2

| | | | Number and | Number and | |
|---|---|---|---|---|---|
| | Amino acid | Nucleotide | sequence of | sequence of | |
| Name | sequence | sequence | primers(Fw) | primers(Rev) | Template |
| CCA36173Cterdel | SEQ ID NO: 108 | SEQ ID NO: 65 | 15 (SEQ ID NO: 25) | 43 (SEQ ID NO: 66) | pUC-PgapCCA |
| | | | | | 36173T38473Zeo |
| CCA37695Cterdel | SEQ ID NO: 109 | SEQ ID NO: 67 | 17 (SEQ ID NO: 28) | 44 (SEQ ID NO: 68) | pUC-PgapCCA |
| | | | | | 37695T38473Zeo |
| CCA41161Cterdel | SEQ ID NO: 110 | SEQ ID NO: 69 | 19 (SEQ ID NO: 31) | 45 (SEQ ID NO: 70) | pUC-PgapCCA |
| | | | | | 41161T38473Zeo |
| CCA41167Cterdel | SEQ ID NO: 111 | SEQ ID NO: 71 | 21 (SEQ ID NO: 34) | 46 (SEQ ID NO: 72) | pUC-PgapCCA |
| | | | | | 41167T38473Zeo |
| CCA37701Cterdel | SEQ ID NO: 112 | SEQ ID NO: 73 | 23 (SEQ ID NO: 37) | 47 (SEQ ID NO: 74) | pUC-PgapCCA |
| | | | | | 37701T38473Zeo |
| CCA40175Cterdel | SEQ ID NO: 113 | SEQ ID NO: 75 | 25 (SEQ ID NO: 40) | 48 (SEQ ID NO: 76) | pUC-PgapCCA |
| | | | | | 40175T38473Zeo |
| CCA37509Cterdel | SEQ ID NO: 114 | SEQ ID NO: 77 | 27 (SEQ ID NO: 43) | 49 (SEQ ID NO: 78) | pUC-PgapCCA |
| | | | | | 37509T38473Zeo |
| CCA38967Cterdel | SEQ ID NO: 115 | SEQ ID NO: 79 | 29 (SEQ ID NO: 46) | 50 (SEQ ID NO: 80) | pUC-PgapCCA |
| | | | | | 38967T38473Zeo |
| CCA37504Cterdel | SEQ ID NO: 116 | SEQ ID NO: 81 | 31 (SEQ ID NO: 49) | 51 (SEQ ID NO: 82) | pUC-PgapCCA |
| | | | | | 37504T38473Zeo |
| CAY67126Cterdel | SEQ ID NO: 117 | SEQ ID NO: 83 | 33 (SEQ ID NO: 52) | 52 (SEQ ID NO: 84) | pUC-PgapCAY |
| | | | | | 67126T38473Zeo |
| CAY68445Cterdel | SEQ ID NO: 118 | SEQ ID NO: 85 | 35 (SEQ ID NO: 55) | 53 (SEQ ID NO: 86) | pUC-PgapCAY |
| | | | | | 68445T38473Zeo |
| CAY68608Cterdel | SEQ ID NO: 119 | SEQ ID NO: 87 | 37 (SEQ ID NO: 58) | 54 (SEQ ID NO: 88) | pUC-PgapCAY |
| | | | | | 68608T38473Zeo |
| CAY71233Cterdel | SEQ ID NO: 120 | SEQ ID NO: 89 | 39 (SEQ ID NO: 61) | 55 (SEQ ID NO: 90) | pUC-PgapCAY |
| | | | | | 71233T38473Zeo |

In each of these nucleic acid fragments, a GAP promoter sequence is added, as an overlapping region, upstream of the nucleotide sequence encoding the polypeptide and a CCA38473 terminator sequence is added, as an overlapping region, downstream of the nucleotide sequence encoding the polypeptide.

After treating pUC-PgapT38473Zeo constructed in Example 3 with SpeI, a nucleic acid fragment was prepared by PCR using a Re primer for the GAP promoter (primer 41 (SEQ ID NO: 63)) and a Fw primer for the CCA38473 terminator (primer 42 (SEQ ID NO: (A)). This nucleic acid fragment was mixed with the nucleic acid fragment prepared by PCR that has the above-mentioned nucleotide sequence encoding the C-terminally deleted polypeptide. Then, these fragments were linked together by using a Gibson Assembly system from New England Biolabs Inc., thereby constructing pUC-PgapCCA36173CterdelT38473Zeo, pUC-PgapCCA37695CterdelT38473Zeo, pUC-PgapCCA41161CterdelT38473Zeo, pUC-PgapCCA41167CterdelT38473Zeo, pUC-PgapCCA37701CterdelT38473Zeo, pUC-PgapCCA40175CterdelT38473Zeo, pUC-PgapCCA37509CterdelT38473Zeo, pUC-PgapCCA38967CterdelT38473Zeo, pUC-PgapCCA37504CterdelT38473Zeo, pUC-PgapCAY67126CterdelT38473Zeo, pUC-PgapCAY68445CterdelT38473Zeo, pUC-PgapCAY68608CterdelT38473Zeo, or pUC-PgapCAY71233CterdelT38473Zeo. These vectors are designed to express each C-terminally deleted polypeptide under the control of the GAP promoter.

### Example 6: Generation of Transformed Yeast

The yeast expressing an anti-β galactosidase single-chain antibody obtained in Example 4 was inoculated in 3 ml of YPD medium (1% yeast extract bacto (from Becton, Dickinson and Company), 2% polypeptone (from NIHON PHARMACEUTICAL CO., LTD.), 2% glucose) and cultured with shaking overnight at 30° C. to obtain a preculture medium. 500 μl of the preculture medium thus obtained was inoculated in 50 ml of YPD medium and cultured with shaking up to an OD600 of 1 to 1.5. Then, the yeast was harvested (3000×g, 10 minutes, 20° C.) and resuspended in 10 ml of 50 mM potassium phosphate buffer, pH 7.5 supplemented with 250 μl of 1M DTT (final concentration of 25 mM).

After incubating this suspension for 15 minutes at 30° C., the yeast was harvested (3000×g, 10 minutes, 20° C.) and washed with 50 ml of STM buffer precooled in ice (270 mM sucrose, 10 mM Tris-HCl, 1 mM magnesium chloride, pH7.5). After harvesting the yeast from the washing solution (3000×g, 10 minutes, 4° C.) and washing again with 25 ml of STM buffer, the yeast was harvested (3000×g, 10 minutes, 4° C.). Finally, the obtained yeast was suspended in 250 μl of the ice cold STM buffer and this suspension was used as a competent cell solution.

Some of the expression vectors of C-terminally deleted polypeptides constructed in Example 5 were selected randomly and used to transform E. coli. The transformant obtained was cultured in 5 ml of 2YT medium containing ampicillin (1.6% tryptone bacto (from Becton, Dickinson and Company), 1% yeast extract bacto (from Becton, Dickinson and Company), 0.5% sodium chloride, 0.01% ampicillin sodium (from FUJIFILM Wako Pure Chemical Corporation)). Each of the expression vectors of C-terminally deleted polypeptides was obtained from the resulting cell bodies by using a QIAprep spin miniprep kit (from QIAGEN). This plasmid was linearized with NruI treatment by utilizing an NruI recognition sequence within a CCA38473 terminator.

60 μl of the above-mentioned competent cell solution and 1 μl of solution of the linearized expression vector of the C-terminally deleted polypeptide were mixed and transferred into an electroporation cuvette (disposable cuvette electrode, electrode gap of 2 mm (from BM Equipment Co., Ltd.)). After the mixture was subjected to electroporation at 7.5 kV/cm, 25 μF, and 200Ω, cell bodies were suspended in 1 ml of YPD medium and allowed to stand for one hour at 30° C. After allowing to stand for one hour, the yeast was harvested (3000×g, 5 minutes, 20° C.) and 961 μl of the supernatant was discarded. The yeast was resuspended in 100 μl of the remaining solution and then 100 μl of the suspension was spread on a selective YPDZeocin™ agar plate (1% yeast extract bacto (from Becton, Dickinson and Company), 2% polypeptone (from NIHON PHARMACEUTICAL CO., LTD.), 2% glucose, 1.5% agarose, 0.01% Zeocin™ (from Thermo Fisher Scientific Inc.). A strain that grew in static culture for 3 days at 30° C. was selected to obtain a yeast expressing both the anti-β galactosidase single-chain antibody and the C-terminally deleted polypeptide.

### Example 7: Culture of Transformed Yeast

Komagataella pastoris strain ATCC76273, or the yeast expressing an anti-β galactosidase single-chain antibody, the yeast expressing a polypeptide, the yeast expressing an anti-β galactosidase single-chain antibody and a polypeptide, or the yeast expressing an anti-β galactosidase single-chain antibody and a C-terminally deleted polypeptide, which were obtained in Examples 4 and 6, was inoculated in 2 ml of BMMY medium (1% yeast extract bacto (from Becton, Dickinson and Company), 2% polypeptone (from NIHON PHARMACEUTICAL CO., LTD.), 0.34% yeast nitrogen base Without Amino Acid and Ammonium Sulfate (from Becton, Dickinson and Company), 1% Ammonium Sulfate, 0.4 mg/l Biotin, 100 mM potassium phosphate (pH6.0), 2% Methanol). After culturing with shaking at 170 rpm for 72 hours at 30° C., culture supernatant was collected by centrifugation (12000 rpm, 5 minutes, 4° C.). The cell body concentration was determined based on OD600.

### Example 8: Measurement of Amount of Protein Secreted in Culture Supernatant by Bradford Assay

The expression level of proteins secreted into culture supernatant in Example 7 was determined by a Bradford assay as described below.

300 μl of culture supernatant of Komagataella pastoris strain ATCC76273 and 300 μl of culture supernatant of a yeast expressing a polypeptide were applied onto a centrifugal filter (Amicon Ultra-0.5, PLGC Ultracel-10 membrane, 10 kDa (from Merck Millipore)) and centrifuged (14000×g, 20 minutes, 4° C.). The flow-through was discarded and 300 μl of PBS buffer (8 g/L sodium chloride, 0.2 g/L potassium chloride, 1.15 g/L sodium hydrogenphosphate (anhydrous), 0.2 g/L potassium dihydrogenphosphate (anhydrous)) was added to the centrifugal filter. After washing the filter, centrifugation was performed (14000×g, 20 minutes, 4° C.). The flow-through was discarded and 300 μl of PBS buffer was added to the centrifugal filter. After washing the filter, centrifugation was performed (14000×g, 20 minutes, 4° C.). The filter was inverted and placed in a new tube and centrifugation was performed (1000×g, 2 minutes, 4° C.) to recover the remaining solution of the secreted protein. PBS buffer was added to this solution of the secreted protein to make 300 μl.

Serially diluted standard bovine serum albumin and diluted solution of the secreted protein were added to wells of a microplate (a cell culture plate from TPP) at 150 μL/well. 150 μl of 1× dye reagent (Quick Start Protein Assay from Bio-Rad Laboratories, Inc.) was added thereto and allowed to react for 5 minutes at room temperature. Then, the absorbance at 595 nm was measured using a microplate reader (Spectra Max Paradigm from Molecular Devices, LLC.). Quantification of the secreted proteins was performed by using a standard curve of the standard bovine serum albumin. The expression level of the secreted proteins determined by this method and their respective cell body concentrations (OD600) were shown in Table 3.

In Table 3, control (1) represents the results when the culture supernatant of Komagataella pastoris strain ATCC76273 was used and (2) to (14) represents the results when the culture supernatants of Komagataella pastoris strain ATCC76273 transformed by expression vectors of the respective polypeptides were used.

TABLE 3

| | Source | Total protein | |
|---|---|---|---|
| Expressed protein | of yeast | (mg/L) | OD600 |
| 1. Control (Strain ATCC76273) | Example 4 | 30.3 | 34.1 |
| 2. CCA36173 (SEQ ID NO: 95) | Example 4 | 88.6 | 33.6 |
| 3. CCA37695 (SEQ ID NO: 96) | Example 4 | 69.2 | 34.8 |
| 4. CCA41161 (SEQ ID NO: 97) | Example 4 | 88.7 | 27.7 |
| 5. CCA41167 (SEQ ID NO: 98) | Example 4 | 76.4 | 34.1 |
| 6. CCA37701 (SEQ ID NO: 99) | Example 4 | 56.0 | 36.2 |
| 7. CCA40175 (SEQ ID NO: 100) | Example 4 | 80.8 | 32.8 |
| 8. CCA37509 (SEQ ID NO: 101) | Example 4 | 36.3 | 35.2 |
| 9. CCA38967 (SEQ ID NO: 102) | Example 4 | 42.5 | 34.1 |
| 10. CCA37504 (SEQ ID NO: 103) | Example 4 | 74.6 | 33.6 |
| 11. CAY67126 (SEQ ID NO: 104) | Example 4 | 67.3 | 35.7 |
| 12. CAY68445 (SEQ ID NO: 105) | Example 4 | 116.4 | 31.6 |
| 13. CAY68608 (SEQ ID NO: 106) | Example 4 | 67.4 | 31.7 |
| 14. CAY71233 (SEQ ID NO: 107) | Example 4 | 86.5 | 34.5 |

As a result, the yeasts expressing polypeptides (2 to 14) clearly showed a higher secretory expression level than Komagataella pastoris strain ATCC76273 (1). This indicates that expression of a polypeptide having an amino acid sequence shown in any of SEQ ID NOs: 95 to 107 improves secretion productivity of endogenous proteins.

### Example 9: Measurement of Amount of Secreted Anti-β Galactosidase Single-Chain Antibody by ELISA Method

The expression level of anti-β galactosidase single-chain antibodies secreted into culture supernatant obtained in Example 7 was determined by a sandwich ELISA assay (Enzyme-Linked Immunosorbent Assay) as described below.

β-galactosidase (5 mg/mL, from F. Hoffmann-La Roche, Ltd.) diluted 2500-fold with an immobilization buffer (8 g/L sodium chloride, 0.2 g/L potassium chloride, 1.15 g/L sodium hydrogenphosphate (anhydrous), 0.2 g/L potassium dihydrogenphosphate (anhydrous), 1 mM magnesium chloride) was added to an ELISA plate (Nunc Immuno Plate Maxisorp (from Thermo Fisher Scientific Inc.)) at 50 μL/well and was incubated overnight at 4° C. After incubation, the solution in the wells was removed and the plate was blocked with 200 μl of ImmunoBlock (from Sumitomo Dainippon Pharma Co., Ltd.) and allowed to stand for one hour at room temperature. After washing with PBST buffer (8 g/L sodium chloride, 0.2 g/L potassium chloride, 1.15 g/L sodium hydrogenphosphate (anhydrous), 0.2 g/L potassium dihydrogenphosphate (anhydrous), 0.1% Tween20) three times, serially diluted standard anti-β galactosidase single-chain antibody and diluted culture supernatant were added at 50 μl/well and allowed to react for one hour at room temperature. After washing with PBST buffer three times, secondary antibody solution diluted 8000-fold in PBST buffer (secondary antibody: Anti-His-tag mAb-HRP-DirecT (from MBL)) was added at 50 μl/well and allowed to react for one hour at room temperature. After washing with PBST buffer three times, 50 μl of TMB-1 Component Microwell Peroxidase Substrate SureBlue (from KPL) was added and allowed to stand for 20 minutes at room temperature. The reaction was stopped by adding 50 μl of TMB Stop Solution (from KPL), and then the absorbance at 450 nm was measured using a microplate reader (Spectra Max Paradigm from Molecular Devices, LLC.). Quantification of the anti-(galactosidase single-chain antibody in the culture supernatant was performed by using a standard curve of the standard anti-β galactosidase single-chain antibody. The secretory expression of anti-β galactosidase single-chain antibodies determined by this method and their respective cell body concentrations (OD600) were shown in Table 4.

In Table 4, control (1) represents the results when the culture supernatant of a yeast expressing an anti-β galactosidase single-chain antibody was used and (2) to (22) represents the results when the culture supernatants of yeasts expressing an anti-β galactosidase single-chain antibody and respective polypeptides were used.

As a result, the yeasts expressing the anti-β galactosidase single-chain antibody and polypeptides (2 to 14) clearly showed a higher secretory expression level than the yeast expressing the anti-β galactosidase single-chain antibody (1). This indicates that expression of a polypeptide having an amino acid sequence shown in any of SEQ ID NOs: 95 to 107 improves secretory expression of heterologous proteins.

Yeasts expressing an anti-β galactosidase single-chain antibody and a C-terminally deleted polypeptide (15 to 22) also showed a higher secretory expression level than the yeast expressing the anti-β galactosidase single-chain antibody (1). However, the secretory expression level tended to be lower compared to the yeasts expressing the anti-β galactosidase single-chain antibody and polypeptides (2 to 14). This suggests that SEQ ID NOs: 108 to 113, 117, and 120, which are N-terminal side sequences of the polypeptides have an effect of improving secretory expression of the proteins and the C-terminal side sequences enhance this effect.

TABLE 4

| | | Single- | |
|---|---|---|---|
| | | chain | |
| | | anti- | |
| | Source | body | |
| Expressed protein | of yeast | (mg/L) | OD600 |
| 1. Control (yeast expressing anti-β | Example 4 | 455.1 | 34.9 |
| galactosidase single-chain antibody) | | | |
| 2. CCA36173 (SEQ ID NO: 95) | Example 4 | 635.4 | 32.2 |
| 3. CCA37695 (SEQ ID NO: 96) | Example 4 | 734.8 | 33.6 |
| 4. CCA41161 (SEQ ID NO: 97) | Example 4 | 688.0 | 33.5 |
| 5. CCA41167 (SEQ ID NO: 98) | Example 4 | 696.4 | 31.8 |
| 6. CCA37701 (SEQ ID NO: 99) | Example 4 | 637.1 | 30.6 |
| 7. CCA40175 (SEQ ID NO: 100) | Example 4 | 751.3 | 29.8 |
| 8. CCA37509 (SEQ ID NO: 101) | Example 4 | 553.2 | 34.6 |
| 9. CCA38967 (SEQ ID NO: 102) | Example 4 | 500.6 | 34.3 |
| 10. CCA37504 (SEQ ID NO: 103) | Example 4 | 515.7 | 31.5 |
| 11. CAY67126 (SEQ ID NO: 104) | Example 4 | 565.8 | 33.3 |
| 12. CAY68445 (SEQ ID NO: 105) | Example 4 | 516.4 | 33.7 |
| 13. CAY68608 (SEQ ID NO: 106) | Example 4 | 533.9 | 33.2 |
| 14. CAY71233 (SEQ ID NO: 107) | Example 4 | 653.3 | 32.7 |
| 15. CCA36173Cterdel (SEQ ID NO: 108) | Example 6 | 592.6 | 33.6 |
| 16. CCA37695Cterdel (SEQ ID NO: 109) | Example 6 | 673.4 | 34.7 |
| 17. CCA41161Cterdel (SEQ ID NO: 110) | Example 6 | 555.8 | 34.4 |
| 18. CCA41167Cterdel (SEQ ID NO: 111) | Example 6 | 572.7 | 32.6 |
| 19. CCA37701Cterdel (SEQ ID NO: 112) | Example 6 | 583.7 | 34.2 |
| 20. CCA40175Cterdel (SEQ ID NO: 113) | Example 6 | 568.5 | 32.6 |
| 21. CAY67126Cterdel (SEQ ID NO: 117) | Example 6 | 555.0 | 34.0 |
| 22. CAY71233Cterdel (SEQ ID NO: 120) | Example 6 | 626.1 | 32.8 |

All publications, patents, and patent applications cited herein are herein incorporated by citation in their entireties.

Although the disclosure has been described with respect to only a limited number of embodiments, those skilled in the art, having the benefit of this disclosure, will appreciate that various other embodiments may be devised without departing from the scope of the present invention. Accordingly, the scope of the invention should be limited only by the attached claims.

What is claimed is:

1. A vector comprising a nucleotide sequence selected from the group consisting of:
(a) a nucleotide sequence encoding an amino acid sequence selected from the group consisting of SEQ ID NOs: 113, 108 to 112, and 114 to 120;
(b) a nucleotide sequence encoding an amino acid sequence comprising one or more amino acid substitutions, deletions, and/or additions in the amino acid sequence selected from the group consisting of SEQ ID NOs: 113, 108 to 112, and 114 to 120;
(c) a nucleotide sequence encoding an amino acid sequence having a sequence identity of 85% or more to the amino acid sequence selected from the group consisting of SEQ ID NOs: 113, 108 to 112, and 114 to 120; and
(d) a nucleotide sequence of a nucleic acid that hybridizes under stringent conditions to a nucleic acid consisting of a complementary sequence to a nucleotide sequence encoding the amino acid sequence selected from the group consisting of SEQ ID NOs: 113, 108 to 112, and 114 to 120.

2. The vector according to claim 1, wherein the nucleotide sequences of (a) to (d) are respectively:
(a′) a nucleotide sequence selected from the group consisting of SEQ ID NOs: 75, 65, 67, 69, 71, 73, 77, 79, 81, 83, 85, 87, and 89;
(b′) a nucleotide sequence comprising one or more nucleotide substitutions, deletions, and/or additions in the nucleotide sequence selected from the group consisting of SEQ ID NOs: 75, 65, 67, 69, 71, 73, 77, 79, 81, 83, 85, 87, and 89;
(c′) a nucleotide sequence having a sequence identity of 85% or more to the nucleotide sequence selected from the group consisting of SEQ ID NOs: 75, 65, 67, 69, 71, 73, 77, 79, 81, 83, 85, 87, and 89; and
(d′) a nucleotide sequence of a nucleic acid that hybridizes under stringent conditions to a nucleic acid consisting of a complementary sequence to the nucleotide sequence selected from the group consisting of SEQ ID NOs: 75, 65, 67, 69, 71, 73, 77, 79, 81, 83, 85, 87, and 89.

3. A vector comprising a nucleotide sequence selected from the group consisting of:
(a) a nucleotide sequence encoding an amino acid sequence selected from the group consisting of SEQ ID NOs: 100, 95 to 99, and 101 to 107;
(b) a nucleotide sequence encoding an amino acid sequence comprising one or more amino acid substitutions, deletions, and/or additions in the amino acid sequence selected from the group consisting of SEQ ID NOs: 100, 95 to 99, and 101 to 107;
(c) a nucleotide sequence encoding an amino acid sequence having a sequence identity of 85% or more to the amino acid sequence selected from the group consisting of SEQ ID NOs: 100, 95 to 99, and 101 to 107; and
(d) a nucleotide sequence of a nucleic acid that hybridizes under stringent conditions to a nucleic acid consisting of a complementary sequence to a nucleotide sequence encoding the amino acid sequence selected from the group consisting of SEQ ID NOs: 100, 95 to 99, and 101 to 107.

4. The vector according to claim 3, wherein the nucleotide sequences of (a) to (d) are respectively:
(a′) a nucleotide sequence selected from the group consisting of SEQ ID NOs: 39, 24, 27, 30, 33, 36, 42, 45, 48, 51, 54, 57, and 60;
(b′) a nucleotide sequence comprising one or more nucleotide substitutions, deletions, and/or additions in the nucleotide sequence selected from the group consisting of SEQ ID NOs: 39, 24, 27, 30, 33, 36, 42, 45, 48, 51, 54, 57, and 60;
(c′) a nucleotide sequence having a sequence identity of 85% or more to the nucleotide sequence selected from the group consisting of SEQ ID NOs: 39, 24, 27, 30, 33, 36, 42, 45, 48, 51, 54, 57, and 60; and
(d′) a nucleotide sequence of a nucleic acid that hybridizes under stringent conditions to a nucleic acid consisting of a complementary sequence to the nucleotide sequence selected from the group consisting of SEQ ID NOs: 39, 24, 27, 30, 33, 36, 42, 45, 48, 51, 54, 57, and 60.

5. The vector according to claim 1, wherein the vector increases a secretion amount of a protein in a host cell.

6. A protein secretion enhancer consisting of the vector according to claim 1.

7. A mutant cell having an increased expression of a gene compared to that of a wild-type,
wherein the gene comprises a nucleotide sequence selected from the group consisting of:
(a) a nucleotide sequence encoding an amino acid sequence selected from the group consisting of SEQ ID NOs: 113, 108 to 112, and 14 to 120;
(b) a nucleotide sequence encoding an amino acid sequence comprising one or more amino acid substitutions, deletions, and/or additions in the amino acid sequence selected from the group consisting of SEQ ID NOs: 113, 108 to 112, and 114 to 120;
(c) a nucleotide sequence encoding an amino acid sequence having a sequence identity of 85% or more to the amino acid sequence selected from the group consisting of SEQ ID NOs: 113, 108 to 112, and 114 to 120; and
(d) a nucleotide sequence of a nucleic acid that hybridizes under stringent conditions to a nucleic acid consisting of a complementary sequence to a nucleotide sequence encoding the amino acid sequence selected from the group consisting of SEQ ID NOs: 113, 108 to 112, and 114 to 120, and
wherein the mutant cell has an increased secretion amount of a protein compared to a secretion amount of the wild-type.

8. A cell comprising the vector according to claim 1.

9. The cell according to claim 8, wherein the cell is a yeast, a bacterium, a fungus, an insect cell, an animal cell, or a plant cell.

10. The cell according to claim 9, wherein the cell is the yeast that is selected from the group consisting of a methanol-utilizing yeast, a fission yeast, and a budding yeast.

11. The cell according to claim 10, wherein the cell is the methanol-utilizing yeast that is selected from the group consisting of a yeast belonging to the genus Komagataella or a yeast belonging to the genus Ogataea.

12. A method for preparing a mutant strain, comprising:
introducing a vector into a host cell; and
obtaining a mutant strain comprising the vector,
wherein the mutant strain has an increased secretion amount of a protein compared to a secretion amount of the host cell before the introduction, and
wherein the vector comprises a nucleotide sequence selected from the group consisting of:
(a) a nucleotide sequence encoding an amino acid sequence selected from the group consisting of SEQ ID NOs: 113, 108 to 112, and 114 to 120:
(b) a nucleotide sequence encoding an amino acid sequence comprising one or more amino acid substitutions, deletions, and/or additions in the amino acid sequence selected from the group consisting of SEQ ID NOs: 113, 108 to 112, and 114 to 120;
(c) a nucleotide sequence encoding an amino acid sequence having a sequence identity of 85% or more to the amino acid sequence selected from the group consisting of SEQ ID NOs: 113, 108 to 112, and 114 to 120; and
(d) a nucleotide sequence of a nucleic acid that hybridizes under stringent conditions to a nucleic acid consisting of a complementary sequence to a nucleotide sequence encoding the amino acid sequence selected from the group consisting of SEQ ID NOs: 113, 108 to 112, and 114 to 120.

13. The method according to claim 12, further comprising:
culturing the mutant strain in a culture medium; and
recovering from the culture medium a protein produced by the mutant strain.

14. The method according to claim 13, wherein the protein produced by the mutant strain is a heterologous protein.

15. The method according to claim 13, wherein the culture medium comprises one or more carbon sources selected from the group consisting of glucose, glycerol and methanol.
